(19) United States
(12) Patent Application Publication
(10) Pub. No.: US 2009/0318491 A1
(43) Pub. Date: Dec. 24, 2009

(54) Cytisine and Acetylcholine Analogs and Methods of Treating Mood Disorders

(75) Inventors: Marina Picciotto, Guilford, CT (US); Daniela Gundisch, Hilo, HI (US); Lenka Munoz, New South Wales (AU); Matthias Andra, Bonn (DE); Yann Mineur, New Haven, CT (US)

Correspondence Address: COLEMAN SUDOL SAPONE, P.C., 714 COLORADO AVENUE, BRIDGE PORT, CT, 06605-1601, US

(73) Assignees: Yale Univeristy, New Haven, CT (US); Reheinische Friedrich-Wilhelms- Univeristy Bonn, Bonn (DE)

(21) Appl. No.: 12/086,408

(22) PCT Filed: Jan. 26, 2007
(86) PCT No.: PCT/US07/02297

§ 371 Date: Sep. 11, 2008

Related U.S. Application Data
(60) Provisional application No. 60/763,197, filed on Jan. 27, 2006.

(51) Int. Cl. A61K 31/439; C07D 221/02; C07C 269/00; C07D 471/08; A61K 31/27

(52) U.S. Cl. 514/292; 546/101; 560/24; 546/81; 514/290; 514/478

(57) ABSTRACT

The present invention relates to compounds which are derivatives of cytisine or acetylcholine which exhibit activity as agonists, partial agonists or antagonists of nicotinic acetylcholine receptors and may be used in modulating these receptors and in treating mood disorders in patients in need of therapy.

### RELATED APPLICATIONS

This application claims priority from U.S. provisional application no. U.S. Ser. No. 60/763,197, filed Jan. 27, 2006, entitled “Novel Nicotine Receptor Partial Agonists”, which is incorporated by reference in its entirety herein.

This invention was made with support from the United States government in grant no. NIH AA13334. The Government retains certain rights in the invention.

### FIELD OF THE INVENTION

The present invention relates to compounds which exhibit activity as agonists, partial agonists or antagonists of nicotinic acetylcholine receptors and their use in modulating these receptors and in treating mood disorders in patients in need of therapy.

### BACKGROUND OF THE INVENTION

A number of observations suggest that smoking and nicotine can regulate mood in both human subjects and animal models. In clinical studies, depressed subjects have an approximately 50% higher incidence of smoking than the general population (Diwan et al., Schizophr Res 33, 113-118, 1998; Glassman et al., Jama 264, 1546-1549, 1990; Kessler, Arch Med Res 31, 131-144, 1995). In addition, nicotine patch can reduce symptoms of depression in non-smokers (Salin-Pascual et al., Psychopharmacology (Berl) 121, 476-479, 1995) whereas smoking cessation can exacerbate symptoms of depression (Glassman et al., 1990, supra). Animal studies have also shown that chronic nicotine administration can elicit antidepressant-like effects in rats both in the learned helplessness (Semba et al., Biol Psychiatry, 43, 389-391, 1998) and the forced swim Djuric et al., Physiol Behav, 67, 533-5371999; Tizabi et al., Psychopharmacology (Berl), 142, 193-199, 1999) paradigms. Taken together, these studies suggest that the nicotine in tobacco modulates neuronal systems regulating mood.

Nicotine exerts its effects by binding to, activating and desensitizing nicotinic acetylcholine receptors (nAChRs) in the central nervous system and autonomic ganglia (Picciotto, Drug Alcohol Depend 51, 165-172, 1998). α4/β2-containing (α4/β2*) nAChRs combined with the α5, α6 or β3 subunits are the most widely expressed nAChRs in the central nervous system, and also have the highest affinity for nicotine, whereas α7*nAChRs form functional homomers and are highly expressed in the hippocampus and cortex, but also found in most other brain regions (Zoli et al., J Neurosci, 18, 4461-4472, 1998). The observation that increased acetylcholine levels results in depression (Janowsky et al., Lancet, 2, 632-635, 1972) whereas nicotine administration can decrease depressive symptoms (Salin-Pascual et al., Psychopharmacology (Berl) 121, 476-479, 1995) appears paradoxical, however, chronic administration of nicotine (particularly as delivered through nicotine patch) can desensitize rather than activate nAChRs (Reitstetter et al., J Pharmacol Exp Ther, 289, 656-660, 1999), resulting in functional antagonism (for reviews see Gentry et al., Curr Drug Targets CNS Neurol Disord 1, 359-385, 2002; Quick et al., Neurobiol., 53, 457-478, 2002). This suggests that blockade rather than activation of nAChRs might have antidepressant effects. This hypothesis is supported by the fact that mecamylamine, a non-selective nAChR antagonist, decreased symptoms of depression in patients with Tourette's syndrome (Dursun et al., Med Hypotheses, 52, 101-109, 1999; Mihailescu et al., Arch Med Res, 31, 131-144, 2000; Salin-Pascual et al., Rev Invest Clin, 55, 677-693, 2003), and has antidepressant-like properties in mice (Caldarone et al, Psychopharmacology (Berl) 170, 94-101, 2004; Rabenstein et al, Psychopharmacology, 189, 395-401, 2006).

Studies in knockout mice have demonstrated that the absence of β2*nAChRs throughout development can lead to antidepressant-like phenotypes (Caldarone et al., Psychopharmacology (Berl) 170, 94-101, 2004). Moreover, amitriptyline, a classical antidepressant, has no effect in these animals, strongly suggesting that β2*nAChRs are involved in the function of a classical antidepressant. This study also showed that subthreshold doses of the nicotinic antagonist mecamylamine and the tricyclic antidepressant amitriptyline could combine to result in antidepressant-like effects, supporting the idea that blockade of nAChRs might be antidepressant. We hypothesized that if blockade of nAChRs results in antidepressant-like behavior, interference with endogenous acetylcholine signaling through these nAChRs might also result in an antidepressant-like response. We therefore used cytisine, a partial agonist of β2*nAChRs and a full agonist at β4 nAChRs (Picciotto et al., Nature, 374, 65-67, 1995) in several tests of antidepressant efficacy. This is of particular interest since recent studies suggest that partial agonists of α4/β2*nAChRs may be useful in smoking cessation (Coe et al., J Med Chem, 48, 3474-3477, 2005). Additionally, we furthered our behavioral analyses by testing locomotor activity and anxiety-like behaviors, because both could be major confounds in the different assessments we used in this study.

### BRIEF DESCRIPTION OF THE FIGURES

FIG. 1 shows generalized antidepressant-like profile of cytisine in three paradigms of antidepressant efficacy.

FIG. 2 shows the decreased c-fos-like immunostaining in several brain regions and strong marking in the nucleus accumbens.

FIG. 3 shows a graph of the total time spent by experimental animals immobile in the tail test after cytosine derivative administration.

### BRIEF DESCRIPTION OF THE INVENTION

The present invention is directed to compounds according to the formula I:

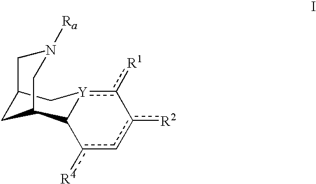

Wherein Rₐ is H, a C₁-C₆ optionally substituted alkyl group, an optionally substituted C₂-C₂₀ acyl (forming an amide) or an optionally substituted carboxyester (forming a urethane with the amine) group;

### Y is C—R₁, or N;

R₁ is absent (such that Y form-s a double bond with the adjacent carbon atom), H, or an optionally substituted C₁₋₃ alkyl, vinyl or alkynyl group;
Each of R¹, R², R³ and R⁴ is independently O, S (such that the O or S forms a double bond with the adjacent carbon atom, preferably R¹ is O), H, NO₂, CN, halogen (F, Br, Cl or I), a C₁-C₆ optionally substituted carboxylic acid group, an optionally substituted O—(C₁-C₆)alkyl (alkoxy), an optionally substituted S—(C₁-C₆)alkyl (thioether), an optionally substituted C₁-C₁₂ hydrocarbyl, an optionally substituted aryl, an optionally substituted heteroaryl, an optionally substituted heterocycle, an optionally substituted —C(O)—(C₁-C₆) alkyl (ketone), an optionally substituted —C(O)—O—(C₁-C₆) alkyl (ester), an optionally substituted O—C(O)—(C₁-C₆) alkyl (ester), an optionally substituted —C(O)—NH(C₁-C₆) alkyl (urea), an optionally substituted —C(O)—N(C₁-C₆)dialkyl, an optionally substituted —C(O)—NH(aryl), an optionally substituted —C(O)—N(diaryl), an optionally substituted —C(O)—NH(heteroaryl), an optionally substituted —C(O)—N(diheteroaryl), an optionally substituted —C(O)—NH(heterocycle), an optionally substituted —C(O)—N(diheterocycle), an optionally substituted —NHC(O)—(C₁-C₆)alkyl, an optionally substituted —NHC(O)-aryl, an optionally substituted —NHC(O)-heteroaryl or an optionally substituted —NHC(O)-heterocycle) with the proviso that not more than two of R¹, R², R³ and R⁴ is O or S (thus forming a double bond with the adjacent carbon atom, and pharmaceutically acceptable salts, solvates and polymorphs thereof.

In preferred compounds according to the present invention at least one of R², R³ or R⁴ is other than H and Rₐ is H, an acyl group or carboxyester group. In other preferred aspects of the invention, Rₐ is other than H.

Preferably, in compounds according to the present invention Rₐ is H or an acyl group (for prodrug advantages), Y is N, R¹ is O, R² is other than H (preferably an optionally substituted aryl or heteroaryl group, and R³ is H or a halogen (preferably F or Cl). Preferably R² and R⁴ are H, an optionally substituted phenyl or heterocyclic group, preferably a meta-substituted phenyl or meta-substituted benzyl, an optionally substituted morpholine, piperidine, piperazine, a furanyl group (especially a 2-furanyl or 3-furanyl group), a thienyl (thiophene, especially 2-thienyl or 3-thienyl group), an optionally substituted indole, optionally substituted benzofuran, optionally substituted benzofurazan, optionally substituted pyridine (especially 2-pyridyl or 3-pyridyl), optionally substituted quinoline, optionally substituted imidazole or an optionally substituted pyrazole group.

In other preferred compounds according to the present invention, Ra is H or methyl (preferably H), Y is N, R¹ is O (forming a keto group with the adjacent carbon atom), R² is a an optionally substituted carbocyclic or heterocyclic group, preferably an optionally substituted aryl or heteroaryl group, more preferably an optionally substituted phenyl, indole, benzofuran, pyridine (substituted at the 2 or 3 position of the pyridine group), quinoline, imidazole or pyrazole group.

In certain preferred embodiments R¹ R² and R³ are H, and R⁴ is an optionally substituted alkyl (preferably, methyl), an optionally substituted vinyl-containing group or an optionally substituted phenyl or heteroaryl group.

In certain preferred aspects of the invention R² and/or R⁴ is a group according to the structure

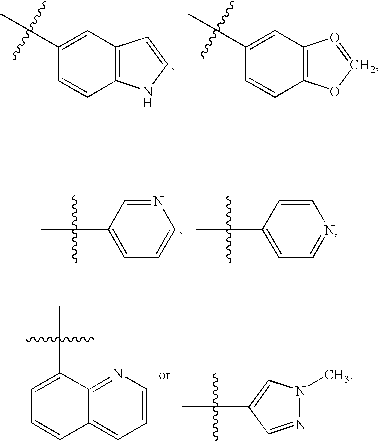

In alternative embodiments according to the present invention, the preferred compound is a compound according to formula II, below:

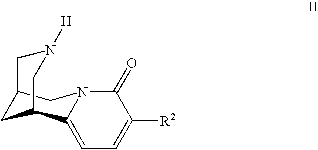

Where R² is an optionally substituted group according to the structure:

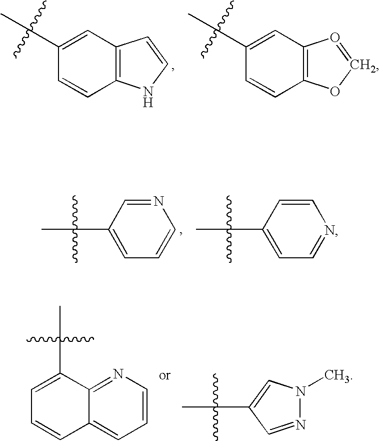

Two particularly preferred compounds according to the present invention are

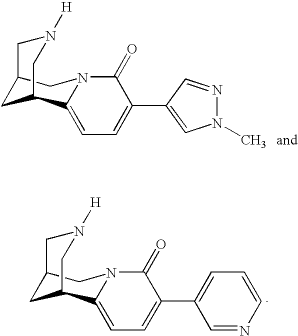

In alternative embodiments, the present invention relates to a compound according to formula III:

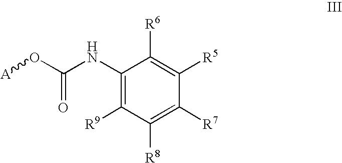

Where A is a 5 to 9-membered substituted azacyclic or azabicyclic group, an —NR^{1a}R^{2a} or a —NR^{1a}R^{2a}R^{3a}+ group;
R^{1a}R^{2a} and R^{3a} are each independently H or an optionally substituted C₁-C₃ alkyl group, preferably an optionally substituted methyl group, more preferably a methyl group;
R⁵ and R⁸ are each independently selected from H, NO₂, CN, halogen (F, Br, Cl or I), a C₁-C₆ optionally substituted carboxylic acid group, an optionally substituted O—(C₁-C₆)alkyl (alkoxy), an optionally substituted S—(C₁-C₆)alkyl (thioether), an optionally substituted C₁-C₁₂ hydrocarbyl, an optionally substituted aryl, an optionally substituted heteroaryl, an optionally substituted heterocycle, an optionally substituted —C(O)—(C₁-C₆) alkyl (ketone), an optionally substituted —C(O)—O—(C₁-C₆) alkyl (ester), an optionally substituted O—C(O)—(C₁-C₆) alkyl (ester), an optionally substituted —C(O)—NH(C₁-C₆) alkyl (urea), an optionally substituted —C(O)—N(C₁-C₆)dialkyl, an optionally substituted —C(O)—NH(aryl), an optionally substituted —C(O)—N(diaryl), an optionally substituted —C(O)—NH(heteroaryl), an optionally substituted —C(O)N(diheteroaryl), an optionally substituted —C(O)—NH(heterocycle), an optionally substituted —C(O)—N(diheterocycle), an optionally substituted —NHC(O)—(C₁-C₆)alkyl, an optionally substituted —NHC(O)-aryl, an optionally substituted —NHC(O)-heteroaryl or an optionally substituted —NHC(O)-heterocycle);
R⁶, R⁷ and R⁹ are each independently H, NO₂, CN, halogen (preferably F, Br or Cl) or an optionally substituted C₁-C₁₂ hydrocarbyl group or an optionally substituted aryl (preferably phenyl) group, or a pharmaceutically acceptable salt, solvate or polymorph thereof.

In preferred aspects of the invention, A is connected to the remaining molecule either directly or through a methylene group. In further preferred aspects of the invention, A is

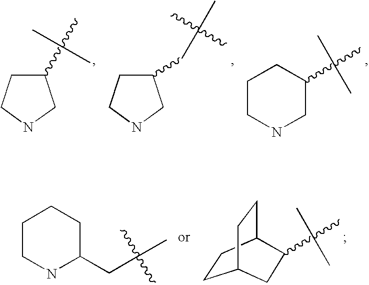

R⁵ and R⁸ are each independently selected from H, halogen (preferably, F or Br), an optionally substituted C₁-C₄ alkyl, or an optionally substituted phenyl group (which includes an optionally substituted styryl group or a F- or Br-substituted phenyl group) and R⁶, R⁷ and R⁹ are each independently H, a halogen (preferably F, Cl or Br) or an optionally substituted methyl group (CH₃ or CF₃). Preferably, R⁶, R⁷ and R⁹ are H or F, Br, most preferably H.

In further preferred aspects A is

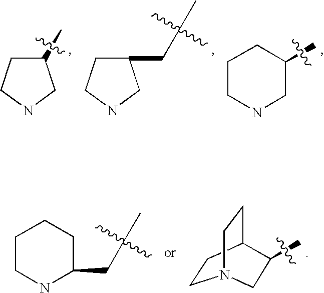

The present invention also relates to pharmaceutical compositions comprising an effective amount of at least one compound as otherwise described hereinabove, optionally in combination with a pharmaceutically acceptable carrier, additive or excipient.

The present invention also relates to methods of using the above-described compounds to modulate nicotinic receptors in a patient or subject, in particular, nicotinic acetylcholine receptors (NChRs), especially α4β2 nAChR, α3β4 nAChR and/or α7 nAChR. Through modulation of these and/or other receptors, compounds according to the present invention may be used in effective amounts to treat a patient or subject in need thereof for one or more of the following mood or affective disorders: major depressive disorder, bipolar disorder, unipolar disorder, dysthymia (dysthymic disorder), post-partum depression, seasonal affective disorder and schizoaffective disorder, among others.

### DETAILED DESCRIPTION OF THE INVENTION

The following terms shall be used to describe the invention. In instances where a term is not defined explicitly or implicitly herein, such term is given its common meaning by those in the relevant art. In certain instances, certain terms may subsume other terms and such use is to be interpreted within context to avoid redundancy.

The term “patient” or “subject” refers to a mammal, preferably a human, in need of treatment or therapy to which compounds according to the present invention are administered in order to treat a condition or disease state modulated through the binding of a compound according to the present invention with a receptor, and in particular, a nicotinic receptor.

The term “compound”, as used herein, unless otherwise indicated, refers to any specific chemical compound disclosed herein and includes in context, tautomers, regioisomers (especially cis/trans), geometric isomers, and where applicable, optical isomers thereof, as well as pharmaceutically acceptable salts, solvates and polymorphs thereof. Within its use in context, the term compound generally refers to a single compound, but also may include other compounds such as stereoisomers, regioisomers and/or optical isomers (including in some instances, racemic mixtures) as well as specific enantiomers or enantiomerically enriched mixtures of disclosed compounds. The compounds of this invention include all stereoisomers where relevant (e.g., cis and trans isomers) and all optical isomers of the present compounds (eg., R and S enantiomers), as well as racemic, diastereomeric and/or other mixtures of such isomers, as well as all pharmaceutically acceptable salt forms, solvates, polymorphs and prodrug forms of the present compounds, where applicable.

The term “effective” is used to describe an amount or concentration of a compound, intermediate or component which, when used in context, over a period of time produces an intended result, whether that result is modulation of a nicotinic acetylcholine receptor, or the treatment of a mood disorder, or as otherwise specified herein.

The term “nicotinic acetylcholine receptors” or “NAChRs” are used to describe ionotropic receptors that form ion channels in cells' plasma membranes. Like the other type of acetylcholine receptors, muscarinic acetylcholine receptors, their opening is triggered by the neurotransmitter acetylcholine, but they are also opened by nicotine. Their action is inhibited by curare. Nicotinic acetylcholine receptors are present in many tissues in the body. The neuronal receptors are found in the central nervous system and the peripheral nervous system. The neuromuscular receptors are found in the neuromuscular junctions of somatic muscles; stimulation of these receptors causes muscular contraction.

Nicotinic receptors, with a molecular weight of about 280 kDa, are made up of five receptor subunits, arranged symmetrically around the central pore. Twelve types of nicotinic receptor subunits, α2 through 10 and β2 through 4, combine to form pentamers. The subunits are somewhat similar to one another, especially in the hydrophobic regions. The muscle form of the nAChR consist of two α subunits, a β, a δ and either a γ or an ∈. The neuronal forms are much more heterogeneous, with a wide range of possible subunit combinations.

Neuronal nAChRs are much more diverse than the muscle type because many subunit combinations are possible. The assembly of the subunits in the neuronal nAChR is less tightly constrained than that of the muscle receptor. To date, nine neuronal subunits with the homology of muscle α1 (α2-α10) and three non-α subunits (β2-β4) have been identified. (See, Lindstrom J. “The structures of neuronal nicotinic receptors” In: Clementi, F.; Fornasari D.; Gotti, C. (Eds.), Handbook of Experimental Pharmacology Vol. Neuronal Nicotinic receptors. Springer, Berlin, pp. 101-162). In CNS these subunits form either heteromeric or homomeric complexes. The majority of the heteromeric receptor complexes identified are believed to contain a single type of α and a single type β subunit in (α)₂(β)₃ stochiometry, e.g. (α4)₂(β2)₃. However, heteromeric receptors involving three types of subunits can be formed as well, e.g. the α3 and α5 subunits have been shown to form “triplet” receptors when co-expressed with other α or β subunits in the Xenopus expression system. The properties of these triple receptors were distinct from those containing a single type of α and β subunit. The functional homomeric nAChR pentamers can be composed only of α7-α10 subunits (e.g. (α7)₅, the homomeric subtype widely distributed in mammalian CNS). In the present invention, while compounds according to the invention may bind generally to nAChRs, the focus of activity is on the nAChRs which are labeled as α4β2 nAChR, α3β4 nAChR and/or α7 nAChR.

The term “modulate” means, with respect to disease states or conditions modulated through binding of a compound according to the present invention to nicotine nAChR receptors to produce, either directly or indirectly, an improvement or lessening of a condition or disease state which was, prior to administration of a compound according to the present invention, sub-optimal and in many cases, debilitating or in some instances, even life threatening. Modulation may occur by virtue of agonist activity, antagonist activity or mixed agonist/antagonist activity (partial agonist activity), depending on the receptor site and the compound.

The term “independently” is used herein to indicate that the variable, which is independently applied, varies independently from application to application.

The term “non-existent” or “absent” refers to the fact that a substituent is absent and the group to which such substituent is attached forms an additional bond with an adjacent atom or group.

The term “optionally substituted” means optional substitution with the specified groups, radicals or moieties. It should be noted that any atom with unsatisfied valences in the text, schemes, examples and tables, etc. herein is assumed/interpreted to have the hydrogen atom(s) to satisfy the valences.

The term “coadministration” or “combination therapy” is used to describe a therapy in which at least two active compounds in effective amounts are used to treat one or more mood disorder or combination of a mood disorder and another disorder as otherwise described hereunder. Although the term coadministration preferably includes the administration of two active compounds to the patient at the same time, it is not necessary that the compounds be administered to the patient at the same time, although effective amounts of the individual compounds will be present in the patient at the same time. Compounds according to the present invention may be administered with one or more traditional agent used to treat mood disorders, including for example, tricyclic antidepressants, such as amitriptyline (Elavil®, Endep®, Tryptanol®, Trepiline®); clomipramine (Anafranil®); desipramine (Norpramin®, Pertofrane®); dothiepin hydrochloride (Prothiaden®, Thaden®); doxepin (Adapin®, Sinequan®); imipramine (Tofranil®); lofepramine (Gamanil®, Lomont®); nortriptyline (Pamelor®); protriptyline (Vivactil®); trimipramine (Surmontil®); MAO inhibitors, such as isocarboxazid (Marplan); phenelzine (Nardil); tranylcypromine (Parnate); as well as selective serotonin reuptake inhibitors (SSRI), including escitalopram oxalate (Lexapro); citalopram (Celexa); fluvoxamine (Luvox); paroxetine (Paxil); sertraline (Zoloft) and fluoxetine (Prozac). Coadministration of one of the present compounds with another agent as otherwise described herein will often result in a synergistic enhancement of the activity of the other agent, an unexpected result. One or more of the present compounds may also be coadministered with another bioactive agent (e.g., antiviral agent, antibiotics, antihyperproliferative disease agent, sedative hypnotic agents, agents which treat inflammatory disease, anticancer agents, analgesic agents, among others or as otherwise described herein), depending upon the desired therapeutic outcome and the disease state or condition treated.

The term “mood disorder” or “affective disorder” is used to describe mental disorders that primarily affect mood and interfere with the activities of daily living. Usually it includes major depressive disorder (MDD) and bipolar disorder (also called Manic Depressive Psychosis), among others.

Mood disorders include, but are not limited to, the following:
Major Depressive Disorder (otherwise known as MDD, major depression or simply depression). This disorder can also exist with psychotic features.
Bipolar disorder, also called bipolar. Bipolar disorder exists as a spectrum of disorders, and can include the following:
   Bipolar I (episodes of depression, and episodes of mania with psychosis)
   Bipolar II (episodes of depression and hypomania)
   Cyclothymia (episodes of dysthymia and hypomania).
Dysthymic Disorder, also called dysthymia.
Post-partum depression. This is the sudden depression which can sometimes occur in a mother after giving birth to a child.
Seasonal affective disorder, also known as SAD. People with this disorder experience depression during the winter months, and may require antidepressants during this time. Another therapy available for use in people with SAD is light therapy.
Schizoaffective disorder, in which the sufferer has experienced the symptoms of schizophrenia as well as symptoms of a mood disorder.

The terms “major depressive disorder”, MDD, major depression and “depression” are used synonymously in the present invention to describe a condition characterized by a long-lasting depressed mood or marked loss of interest or pleasure (anhedonia) in all or nearly all activities. Children and adolescents with MDD may be irritable instead of sad. These symptoms, along with others described below, must be sufficiently severe to interfere significantly with the patient's daily functioning in order for a person to be diagnosed with MDD.

Major depressive disorder is a serious mental disorder that profoundly affects an individual's quality of life. Unlike normal bereavement or an occasional episode of “the blues,” MDD causes a lengthy period of gloom and hopelessness, and may rob the sufferer of the ability to take pleasure in activities or relationships that were previously enjoyable. In some cases, depressive episodes seem to be triggered by an obviously painful event, but MDD develops without a specific identified stressor. Research indicates that an initial episode of depression is likely to be a response to a specific stimulus, but later episodes are progressively more likely to start without a triggering event. A person suffering major depression finds job related responsibilities and such other tasks as parenting burdensome and carried out only with great effort Mental efficiency and memory are affected, causing even simple tasks to be tiring and irritating. Sexual interest dwindles; many people with MDD become withdrawn and avoid any type of social activity. Even the ability to enjoy a good meal or a sound night's sleep is frequently lost; many depressed people report a chronic sense of malaise (general discomfort or unease). For some, the pain and suffering accompanying MDD becomes so unendurable that suicide is viewed as the only option; MDD has the highest mortality rate of any mental disorder.

Major depressive disorder may be limited to a single episode of depression; more commonly, it may become a chronic condition with many episodes of depressed mood. Other symptoms that may develop include psychotic symptoms (bizarre thoughts, including delusional beliefs and hallucinations); catatonia; postpartum onset (sometimes accompanied by psychotic symptoms); and seasonal affective disorder, or SAD.

Such conditions as postpartum depression and seasonal affective disorder accompany MDD only under certain circumstances. Postpartum depression begins within four weeks of giving birth. Women with this disorder experience labile mood (frequent drastic mood changes). They may feel helpless and unable to care adequately for their infant, or they may be completely uninterested in the child. The symptoms of postpartum depression are much more severe than those of the relatively common “new baby blues,” which affect up to 70% of new mothers. The presence of psychotic symptoms in the mother, too many ruminations (obsessive thoughts), or delusions about the infant are associated with a heightened risk of serious harm to the child. The symptoms of postpartum depression are usually attributed to fluctuations in the woman's hormone levels and the emotional impact of bearing a child. The condition is especially likely to occur in women who were highly anxious during pregnancy or had a previous history of mood disorder. Seasonal affective disorder (SAD) is also more common in women than in men; in this case, symptoms of MDD typically begin in fall and winter, especially in northern latitudes in the United States and Canada. Exposure to natural light is limited during the winter in these areas, but the symptoms of SAD typically improve during the spring and summer.

Because MDD is a relatively common mental disorder, researchers have performed a range of different studies to identify possible underlying causes. Three types of causes are commonly identified: intrapsychic, environmental, and biological.

In terms of biological causes, levels of cortisol, a hormone associated with the human “fight-or-flight” response, have long been studied as possible biological markers for depression. In many adults, cortisol levels rise when the person is acutely depressed and return to normal when the depression passes. Research findings have been inconsistent regarding cortisol levels in children and adolescents, although there is some evidence that higher levels of cortisol secretion are associated with more severe depressive symptoms and with a higher likelihood of recurrence. As of 2002, however, cortisol levels were not considered to be reliable enough to be useful in diagnosing MDD and is rarely, if ever, used anymore.

Another biological factor that has been studied in humans are changes in the levels of neurotransmitters, which are chemicals that conduct nerve impulses across the tiny gaps between nerve cells. Variations in the levels of certain neurotransmitters have been researched for many years due to their importance in the brain's limbic system, which is the center of emotions and has many important pathways to other parts of the brain. In depression, the system that regulates a neurotransmitter called serotonin does not function properly. A group of medications known as serotonin specific reuptake inhibitors, or SSRIs, are assumed to be effective in relieving depression because they prevent serotonin from being taken back up too quickly by receptors in the brain.

The symptoms of major depressive disorder include the following. The core symptom of major depression is a sad mood that does not go away. While most people have occasional days when they feel out of sorts, persons with MDD experience low feelings that build gradually over a period of days or weeks. They are usually not able to “snap out of it” even when something positive happens. In some cases, the symptoms are preceded by an obvious loss or painful event, such as divorce or a death in the family, but the disorder may also appear to begin “out of the blue.” People with MDD often appear sad, irritable, and easily moved to tears. They may sleep poorly and complain of vague physical aches and pains; experience sexual difficulties or loss of interest in sex; drop out of social activities; and come across to others as unhappy or lacking in energy. Some people with MDD may deny that they feel depressed, but they lose their enthusiasm for hobbies or work they once found enjoyable and rewarding. Children and adolescents present with many of these same characteristics, but they may often appear easily frustrated and cranky instead of sad. The symptoms of MDD can be summarized as follows:
Disturbed mood (sad, hopeless, discouraged, “down in the dumps”) during most of the day.
Loss of interest or pleasure in activities.
Change in appetite nearly every day, leading either to weight gain or to loss of 5% of body weight. In children, this symptom may appear as a failure to make normal weight gains related to growth.
Insomnia (waking in the middle of the night and having difficulty returning to sleep, or waking too early in the morning) or hypersomnia (sleeping much more than normal).
Psychomotor retardation (slowed thinking, speech, body movements) or agitation (inability to sit still, hand-wringing, pulling at clothing, skin, or other objects) that is apparent to others.
Sense of worthlessness or unreasonable guilt over minor failings.
Problems with clear thinking, concentration, and decision-making.
Recurrent thoughts of death or suicide, or making a suicide attempt.

Recent research indicates that 4.9% of the population of the United States meets the diagnostic criteria for MDD at any given time, but 17.1% will experience at least one episode of the disorder at some point during their lives. While the disorder may affect people at any age, it is most commonly diagnosed in young adults in their twenties.

Depression appears to have become a more common disorder over the past century. Epidemiologists studying the incidence of depression across time compared groups of people born between 1917 and 1936, between 1937 and 1952, and between 1953 and 1966; their results indicated that the rate of depression increased progressively from one generation to the next. While no single explanation for the rise in depressive disorders emerged, some researchers have suggested that the breakdown of social support networks caused by higher rates of family disruption and greater social mobility may be important contributing factors.

Major depressive disorder may be diagnosed when a person visits his or her family doctor with concerns about mood, changes in appetite or sleeping patterns, and similar symptoms. Doctors in family practice, in fact, are more likely to be consulted by patients with depression than doctors in any other medical specialty. In addition, a large proportion of people discuss depressed feelings with their clergyperson, who, in the mainstream Christian and Jewish bodies, has typically been trained to recognize the signs of depression and to encourage the person to see their doctor. In some cases the patient may be brought to see the doctor by a concerned spouse or other family member.

The diagnosis of MDD involves a constellation of symptoms in addition to depressed mood. After taking a careful history, including asking the patient about his or her sleeping patterns, appetite, sex drive, and mood, the doctor will give the patient a physical examination to rule out other possible causes of the symptoms. Certain other disorders may resemble MDD, including cognitive dysfunction caused by the direct effects of a substance (drug of abuse, medication, or toxic chemical); various medical conditions (i.e., an underactive thyroid gland; strokes; or early stages of dementia), or other mental disorders. Such stressful life events as normal bereavement may also produce behaviors similar to those associated with MDD; while a bereaved person may appear to have many of the characteristics of MDD, the disorder would not be diagnosed unless the symptoms continued for more than two months or were extreme in some way. As part of the diagnostic interview, the doctor may give the patient a brief screening questionnaire, such as the Beck Depression Inventory, in order to obtain a clearer picture of the symptoms. In addition to interviewing the patient, the doctor may talk to family members or others who can provide information that the patient may forget, deny, or consider unimportant.

The diagnosis of MDD is complicated by the fact that people with MDD frequently suffer from other mental illnesses at the same time, including anxiety disorders, substance abuse problems, and personality disorders. Given that the patient's symptoms may vary according to age, sex, and stage of the illness, some clinicians have suggested that MDD may actually be a collection or group of disorders with a small number of underlying core symptoms rather than a single entity.

The diagnosis of a person with MDD may also include certain specifiers, including the severity and chronicity of the disorder, the presence of psychotic features (delusions or hallucinations) or catatonia (remaining motionless for long periods of time, and other peculiarities of posture, movement, or speech); melancholia (depressed mood that is worse in the morning; early morning wakening; psychomotor retardation or agitation; significant weight loss; or inappropriate guilt); and information regarding postpartum status. If the depression is currently in remission, this fact is also commonly listed as a diagnostic specifier.

The term “bipolar disorder” is used to describe a mood disorder which is characterized by extreme variations in mood, from mania and/or irritability to depression. Alterations in mood (commonly referred to as “mood swings”) between mania and depression can be frightening and disturbing for persons who have this disorder as well as family members and those people who know and work with them. Manic episodes can be especially distressing because they are often associated with high-risk behaviors like substance abuse, sexual promiscuity, immoderate spending, violent behavior, and disregard for danger. The capacity for empathy is also typically reduced or absent, leaving family members and others without the usual interpersonal protections and understandings that empathy (knowing how our words and actions affect others) provides.

Bipolar II disorder is characterized by recurring episodes of depression and hypomania. Hypomania differs from full mania in the following ways—although expansive and elevated mood states are present, gross lapses of judgment or impulsive behavior tend not to occur. Hypomania does not impair functioning as significantly as mania, and may even be felt as enhancing functioning by the person with the disorder.

### Criteria for Major Depressive Episode and Manic Episode

Major Depressive Episode

Five or more of the following symptoms have been present during the same 2-week period and represent a change from previous functioning; at least one of the symptoms is either (1) depressed mood or (2) loss of interest or pleasure.
Depressed mood most of the day, nearly every day, as indicated by either subjective report (e.g., feels sad or empty) or observation made by others (e.g., appears tearful). Note: In children and adolescents, can be irritable mood.
Markedly diminished interest or pleasure in all, or almost all, activities most of the day, nearly every day (as indicated by either subjective account or observation made by others)
Significant weight loss when not dieting or weight gain (e.g., a change of more than 5% of body weight in a month), or decrease or increase in appetite nearly every day. Note: In children, consider failure to make expected weight gains.
Insomnia or hypersomnia nearly every day
Psychomotor agitation or retardation nearly every day (observable by others, not merely subjective feelings of restlessness or being slowed down)
Fatigue or loss of energy nearly every day
Feelings of worthlessness or excessive or inappropriate guilt (which may be delusional) nearly every day (not merely self-reproach or guilt about being sick)
Diminished ability to think or concentrate, or indecisiveness, nearly every day (either by subjective account or as observed by others)
Recurrent thoughts of death (not just fear of dying), recurrent suicidal ideation without a specific plan, or a suicide attempt or a specific plan for committing suicide

### Manic Episode

A. A distinct period of abnormally and persistently elevated, expansive, or irritable mood, lasting at least 1 week (or any duration if hospitalization is necessary)

B. During the period of mood disturbance, three (or more) of the following symptoms have persisted (four if the mood is only irritable) and have been present to a significant degree:

Inflated self-esteem or grandiosity

Decreased need for sleep (e.g., feels rested after only 3 hours of sleep)

More talkative than usual or pressure to keep talking

Flight of ideas or subjective experience that thoughts are racing

Distractibility (i.e., attention too easily drawn to unimportant or irrelevant external stimuli)

Increase in goal-directed activity (either socially, at work or school, or sexually) or psychomotor agitation

Excessive involvement in pleasurable activities that have a high potential for painful consequences (e.g., engaging in unrestrained buying sprees, sexual indiscretions, or foolish business investments)

The term “dysthymic disorder” is used to describe a mood disorder characterized by a variety of depressive symptoms in a patient for at least two years, and these symptoms are not numerous or severe enough to qualify for major depressive disorder. It can be difficult to distinguish from major depressive disorder, since it is similar in terms of the types of symptoms present, and their onset and duration historically. In both disorders, individuals may have changes in their sleep patterns or appetite, low energy or fatigue, low self-esteem, poor concentration or difficulty making decisions, or hopelessness during periods of depressed mood. However, individuals with dysthymic disorder may have more prominent cognitive or interpersonal symptoms, such as pessimism, feelings of inadequacy, and social withdrawal.

Dysthymic disorder often has its onset during teen years or early adulthood. When this occurs, it may negatively affect personality development, since the feelings of inadequacy and social withdrawal can interfere with achieving the important social goals of that time. As a consequence, persons with dysthymic disorder may be more likely to remain single and those with early onset (before age 21) more likely to develop personality disorders than those with later onset.

When there is this early onset, individuals may feel that the depression is “just the way life is,” since they have never known a period of better mood and pleasure as teens or adults. As a result, many do not seek treatment until, for some reason, the depression becomes more severe. This happens fairly often, as, each year, about 10% of those with dysthymic disorder develop major depressive disorder. Many persons with dysthymic disorder report that they have been depressed for decades before they finally seek treatment. Like major depressive disorder, dysthymic disorder can cause significant impairments in occupational, academic, social, or recreational functioning.

Treatment choices are also fairly similar to those used for major depressive disorder, though this has not been as well studied. There is some evidence that psychotherapy, in the forms of cognitive-behavioral therapy or interpersonal therapy, may be helpful. However, many believe that anti-depressant medications are the preferred treatment, especially for those individuals who, as is often the case in this disorder, have had one or more prior unsuccessful trials of psychotherapy. There is reason to believe that treatment with a combination of psychotherapy and medication may be better for some patients, such as those with significant psychosocial stressors, marital problems, residual symptoms, or other maladaptive cognitive or behavioral habits.

It is not absolutely clear that dysthymic disorder is actually separate from major depressive disorder. It may only differ in terms of severity and the course of the illness. Several factors suggest that the two disorders may, in fact, share some biological basis. These include 1) a similar sex ratio (women are diagnosed with these disorders about twice as often as men), 2) the fact that dysthymic disorder is more common among close relatives of persons with major depressive disorder than in the general population, 3) the high frequency with which those with dysthymic disorder go on to develop major depressive disorder (10% per year, as mentioned above), 4) the presence in some patients in both groups of certain abnormalities in their sleep EEGs, and 5) the similarities in methods of effective treatment.

The term “post-partum depression” is used to describe a period during the first mouth after a delivery (the postpartum period) when a woman may suffer depression During this period a number of major changes for women occur. Their hormones and weight are rapidly readjusting. There may be new and stressful changes in relationships with other children, the father of the baby, parents and in-laws, colleagues at work, and friends. Of course, the new baby needs almost constant attention and feeding every two hours, resulting in sleep deprivation. All of these factors can contribute to post-partum mood swings. If the moodiness only lasts 2-3 weeks and then goes away, it's commonly called the “baby blues”. This natural reaction to stress is experienced by more than half of new mothers. However, if the feelings of depression or anxiety continue more than three weeks, a more serious condition called post-partum depression may exist. About 10% of women experience significant depression after a pregnancy.

A patient has a higher chance of postpartum depression if:
The patient had mood or anxiety disorders prior to pregnancy, including depression with a previous pregnancy
The patient has a close family member who has had depression or anxiety
Anything particularly stressful happened to you during the pregnancy, including illness, death or illness of a loved one, a difficult or emergency delivery, premature delivery, or illness or abnormality in the baby
The patient is in her teens or over age 30
The pregnancy is unwanted or unplanned
The patient abuses alcohol, takes illegal substances, or smokes.

Most of the symptoms are the same as in major depression. In addition to mood fluctuations, the woman becomes preoccupied with the infant's well-being. The intensity of this varies—the preoccupation may become delusional. Women who are depressed may feel withdrawn and unconnected to their baby, and can even feel as if they might harm the baby. The presence of severe or delusional thoughts about the infant are symptoms that need immediate attention. These can be accompanied by psychotic “command hallucinations” to kill the infant or delusions that the infant is possessed.

There is no single test to diagnose post-partum depression. Sometimes depression following pregnancy can be related to other medical conditions. Hypothyroidism, for example, causes symptoms such as fatigue, irritability, and depression. Another clue to this condition can be weight gain or failure to lose weight after pregnancy, despite breast-feeding the baby.

The term “seasonal affective disorder” is used to describe a mood disorder Seasonal affective disorder (SAD) is a form of depression that occurs in relation to the seasons, most commonly beginning in winter. This disorder is marked by symptoms of depression profound enough to seriously affect work and relationships. The disorder may have its onset in adolescence or early adulthood and, like other forms of depression, occurs more frequently in women than in men. Most people with the “winter blahs” or “cabin fever” do not have SAD.

The cause of SAD is not known, but is thought to be related to numerous factors including body temperature, hormone regulation, and ambient light. Light may be a particularly critical feature. A rare form occurs in the summer.

Symptoms can include:
Lack of energy
Depression with the onset of fall or winter
Decreased interest in work or significant activities
Increased appetite with weight gain
Carbohydrate cravings
Increased sleep and excessive daytime sleepiness
Social withdrawal
Afternoon slumps with decreased energy and concentration
Slow, sluggish, lethargic movement

A psychological evaluation rules out other causes for the symptoms and confirms the diagnosis. Light therapy using a special lamp to mimic the spectrum of light from the sun may be helpful. Symptoms often resolve on their own with the change of seasons, although seasonal affective disorder can sometimes progress to a major depressive syndrome.

The term “schizoaffective disorder” is used to describe Schizoaffective disorder is a mental condition that causes both psychosis and mood problems. Psychosis is associated with a loss of contact with reality, hallucinations (hearing voices or seeing things that are not present) and delusions (false, fixed beliefs). Mood disorder problems may include a very bad or very good mood with sleep disturbances, changes in energy and appetite, disrupted concentration, and poor daily function.

The exact cause of schizoaffective disorder is unknown. Genetics and body chemistry may play a role. Schizoaffective disorder is believed to be less common than schizophrenia and mood disorders. Women may have the condition more often than men. While mood disorders are relatively common in children, a full syndrome of schizophrenia is not. Therefore schizoaffective disorder tends to be rare in children.

The signs and symptoms of schizoaffective disorder vary greatly from person to person. Often times, persons with schizoaffective disorder seek treatment for problems with mood, daily function, or intrusive thoughts. Psychosis and mood changes may occur at one time, or off and on by themselves. Psychotic symptoms can persist for at least 2 weeks without significant mood symptoms. The course of the disorder feature cycles of severe symptoms followed by improvement.

The symptoms of schizoaffective disorder include:
Elevated, inflated, or depressed mood
Irritability and poor temper control
Symptoms that could be seen during a manic or depressed state (changes in appetite, energy, sleep)
Hallucinations (particularly auditory hallucinations, “hearing voices”)
Delusions of reference (for example, believing that someone on TV or the radio is speaking directly to you or that secret messages are hidden in common objects)
Paranoia (a feeling that everyone or a particular person or agency is out to get you)
Deteriorating concern with hygiene, grooming
Disorganized and illogical speech

To be diagnosed with schizoaffective disorder, a person must experience psychotic symptoms—but normal mood—for at least 2 weeks. The combination of psychotic and mood symptoms in schizoaffective disorder can be seen in other illnesses such as bipolar disorder. The extreme disturbance in mood is an important part of the schizoaffective disorder Any medical, psychiatric, or drug-related condition that causes psychotic or mood symptoms must be considered and ruled out before a diagnosis of schizoaffective disorder is made. Persons who take steroid medications, have seizure disorders, or who abuse cocaine, amphetamines, and phencyclidine (PCP) can have concurrent schizophrenic and mood disorder symptoms.

The treatment of people with schizoaffective disorder varies. Generally, medications are prescribed to stabilize mood and to treat psychosis. Neuroleptic medications (antipsychotics) are used to treat psychotic symptoms. Lithium may be used to manage mania and to stabilize mood. Anti-seizure medications such as valproic acid and carbamazepine are effective mood stabilizers. These medications may take up to 3 weeks to relieve symptoms. Usually the combination of antipsychotic and mood-stabilizing medication controls both depressive and manic symptoms, but antidepressants may also be needed in some cases. People with schizoaffective disorder have a greater chance of returning to a previous level of functioning than patients with other psychotic disorders. However, long-term treatment is necessary and individual outcomes may vary.

“Hydrocarbon” or “hydrocarbyl” refers to any radical containing carbon and hydrogen, which may be straight, branch-chained or cyclic in nature. Hydrocarbons include linear, branched and cyclic hydrocarbons, including alkyl groups, alkylene groups and unsaturated hydrocarbon groups, which may be optionally substituted. Hydrocarbyl groups may be fully saturated or unsaturated, containing one or more double (“ene”) or triple (“yne”) bonds.

“Alkyl” refers to a fully saturated monovalent hydrocarbyl radical containing carbon and hydrogen, and which may be cyclic, branched or a straight chain. Examples of alkyl groups are methyl, ethyl, n-butyl, n-hexyl, n-heptyl, n-octyl, isopropyl, 2-methylpropyl, cyclopropyl, cyclopropylmethyl, cyclobutyl, cyclopentyl, cyclopentylethyl, cyclohexylethyl and cyclohexyl. Preferred alkyl groups are C₁-C₆ alkyl groups.

“Alkylene” refers to a fully saturated hydrocarbon which is divalent (may be linear, branched or cyclic) and which is optionally substituted. Other terms used to indicate substitutent groups in compounds according to the present invention are as conventionally used in the art. Thus, the term alkylene aryl includes alkylene phenyl such as a benzyl group or ethylene phenyl group, alkylaryl, includes alkylphenyl such a phenyl group which has alkyl groups as substituents, etc. The bond [symbol] , when used in chemical structures of the present application refers to a single chemical bond, which may be an optional double bond, in context (depending upon the substituent).

“Aryl” refers to a substituted or unsubstituted monovalent aromatic radical having a single ring (e.g., benzene) or multiple condensed rings (e.g., naphthyl, anthracenyl, phenanthryl) and can be can be bound to compound according to the present invention at any position on the ring(s). Other examples of aryl groups include heterocyclic aromatic ring systems “heteroaryl” having one or more nitrogen, oxygen, or sulfur atoms in the ring, such as diazole, imidazole, furyl, pyrrole, pyridyl, indole, benzofuran, indole, quinoline and other fused ring systems, among others, which may be substituted or unsubstituted, monocyclic or fused cyclic (having at least two rings).

“Alkoxy” as used herein refers to an alkyl group bound through an ether linkage; that is, an “alkoxy” group may be represented as —O-alkyl where alkyl is as defined above.

The term “cyclic” shall refer to an optionally substituted carbocyclic or heterocyclic group, preferably a 5- or 6-membered ring, but may include 4 and 7-membered rings. “Bicyclic” or “bicyclo” refers to bicyclic group and a fused ring system is comprised of at least two rings each of which are 4 to 7-membered rings, preferably 5-6 membered rings.

The term “heterocycle” or “heterocyclic” shall mean an optionally substituted moiety which is cyclic, contains from 4 to 14 atoms at least one of which is other than a carbon atom, such as a nitrogen, sulfur, oxygen, selenium, phosphorous or other atom. A heterocyclic ring shall contain up to four atoms other than carbon selected from nitrogen, sulfur and oxygen. These rings may be saturated or have unsaturated bonds. Fused rings are also contemplated by the present invention. Preferably, a heterocyclic ring hereunder is a 5- to 7-membered ring or a fused ring system containing two rings, one of which is carbocyclic (such as phenyl). A heterocycle according to the present invention is an optionally substituted imidazole, pyridine, a piperazine (including piperazinone), piperidine, morpholine, pyrollidine, pyrrollidinone, furan (especially, 2-furanyl, 3-furanyl), pyrrole, thiazole, thienyl (thiophene), pyrazine, oxazole, isoxazole, indole, benzofuran, benzofurazan, quinoline, among numerous others, all optionally substituted and may be monocyclic, bicyclic or be represented by two or more fused rings. Preferred heterocycles are preferably unsaturated and include imidazole, pyrazole, diazole, thiophene, pyrrole, pyridine, indole, benzofuran and quinoline, which may be attached at various ring positions. Depending upon its use in context, a heterocyclic ring may be saturated and/or unsaturated. In instances where a heterocyclic ring is fully unsaturated, there may be overlap with the term “heteroaryl”, although when used in certain context, the terms are to be construed to avoid such overlap or redundancy.

The term “unsubstituted” shall mean substituted only with hydrogen atoms. The term “substituted” shall mean, within the chemical context of the compound defined, a substituent (each of which substituents may itself be substituted) selected from a hydrocarbyl (which may be substituted itself, preferably with an optionally substituted alkyl, vinyl or halogen (fluoro) group, among others), preferably an alkyl or vinyl group (generally, no greater than about 12 carbon units in length, preferably no greater than about 6 carbon units in length), an optionally substituted aryl (which also may be heteroaryl and may include an alkylenearyl or alkyleneheteroaryl), an optionally substituted heterocycle (especially including an alkyleneheterocycle), CF₃, halogen (especially fluoro), thiol, hydroxyl, carboxyl, oxygen (to form a keto group), C₁-C₈ alkoxy, CN, nitro, an optionally substituted amine (e.g. an alkyleneamine or a C₁-C₆ monoalkyl or dialkyl amine), C₁-C₈ acyl, C₁-C₈ alkylester, C₁-C₈ alkyleneacyl (keto), C₁-C₈ alkylene ester, carboxylic acid, alkylene carboxylic acid, C₁-C₈ thioester, C₂-C₈ ether, C₁-C₈ thioether, amide (amido or carboxamido), substituted amide (especially mono- or di-alkylamide) or alkyleneamide, an optionally substituted carbamate or urethane group, wherein an alkylene group or other carbon group not otherwise specified contains from 1 to 8 carbon units long (alternatively, about 2-6 carbon units long) and the alkyl group on an ester group is from 1 to 8 carbon units long, preferably up to 4 carbon units long. Various optionally substituted moieties may be substituted with 5 or more substituents, preferably no more than 3 substituents and preferably from 1 to 3 substituents.

The term “geometric isomer” shall be used to signify an isomer of a compound according to the present invention wherein a chemical group or atom occupies different spatial positions in relation to double bonds or in saturated ring systems having at least three members in the ring as well as in certain coordination compounds. Thus “cis” and “trans” isomers are geometric isomers as well as isomers of for example, cyclohexane and other cyclic systems. In the present invention all geometric isomers as mixtures (impure) or pure isomers are contemplated by the present invention. In preferred aspects, the present invention is directed to pure geometric isomers.

The term “optical isomer” is used to describe either of two kinds of optically active 3-dimensional isomers (stereoisomers). One kind is represented by mirror-image structures called enantiomers, which result from the presence of one or more asymmetric carbon atoms. The other kind is exemplified by diastereomers, which are not mirror images and which contain at least two asymmetric carbon atoms. Thus, such compounds have 2ₙ optical isomers, where n is the number of asymmetric carbon atoms. In the present invention all optical isomers in impure (i.e., as mixtures) or pure or substantially pure form (such as enantiomerically enriched or as separated diastereomers) are contemplated by the present invention. In certain aspects, the pure enantiomer or diastereomer is the preferred compound.

The present invention includes the compositions comprising the pharmaceutically acceptable salt. i.e., the acid or base addition salts of compounds of the present invention and their derivatives. The acids which may be used to prepare the pharmaceutically acceptable acid addition salts of the aforementioned base compounds useful in this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among others.

Pharmaceutically acceptable base addition salts may also be used to produce pharmaceutically acceptable salt forms of the compounds according to the present invention. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (e, calcium and magnesium), ammonium or water-soluble amine addition salts such as N-methylglucamine-(meglumine), and the lower alkanolammonium and other base salts of pharmaceutically acceptable organic amines, among others.

Compounds according to the present invention are primarily active for their modulation (agonist, partial agonist or antagonist activity, preferably partial agonist activity) of the nicotine receptor (nicotinic acetylcholine receptors or NChRs, especially α4β2 nAChR, α3β4 nAChR and/or α7 nAChR in patients or subjects. Regardless of the mechanism, the compounds of the present invention may be used to treat disease states or conditions in patients or subjects who suffer from those conditions or disease states or are at risk for those conditions. In this method a compound in an effective amount is administered to a patient in need of therapy to treat the condition(s) or disease state(s). These disease states and conditions include, for example, mood disorders, including such disorders as major depressive disorder, bipolar disorder, unipolar disorder, dysthymia (dysthymic disorder), post-partum depression, seasonal affective disorder and schizoaffective disorder, among others.

Compositions according to the present invention may be administered by any conventional means known in the art. Pharmaceutical formulations include those suitable for oral, rectal, nasal, topical (including buccal and sublingual), vaginal or parenteral (including intramuscular, sub-cutaneous and intravenous) administration. Compositions according to the present invention may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. When desired, the above described formulations may be adapted to provide sustained release characteristics of the active ingredient(s) in the composition using standard methods well-known in the art.

In the pharmaceutical aspect according to the present invention, the compound(s) according to the present invention is formulated preferably in admixture with a pharmaceutically acceptable carrier. In general, it is preferable to administer the pharmaceutical composition orally, but certain formulations may be preferably administered parenterally and in particular, in intravenous or intramuscular dosage form, as well as via other parenteral routes, such as transdermal, buccal, subcutaneous, suppository or other route, including via inhalation intranasally. Oral dosage forms are preferably administered in tablet or capsule (preferably, hard or soft gelatin) form. Intravenous and intramuscular formulations are preferably administered in sterile saline. Of course, one of ordinary skill in the art may modify the formulations within the teachings of the specification to provide numerous formulations for a particular route of administration without rendering the compositions of the present invention unstable or compromising their therapeutic activity.

Compositions suitable for parenteral injection may comprise physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants.

These compositions may also contain adjuvants such as preserving, wetting, emulsifying, and/or dispersing agents. Prevention of microorganism contamination of the compositions can be accomplished by the addition of various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and/or gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coatings or shells, such as enteric coatings and others well known in the art. They may also contain opacifying agents, and can also be of such composition that they release the active compound or compounds in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, sesame seed oil, glycerol, tetrahydrofurfuryl alcohol, polyethylene glycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

Suspensions, in addition to the active compound, may contain suspending agents, as for example, ethoxylated isostearyl alcohols, polyoxyethylene sorbitol or sorbitan esters, microcrystalline cellulose, aluminum metahydroxide, bentonite, agar-agar, or tragacanth, or mixtures of these substances, and the like.

Compositions for rectal or vaginal administration, where applicable, can be prepared by mixing an active agent and any additional compounds with suitable non-irritating excipients or carriers such as cocoa butter, polyethylene glycol or a suppository wax, which are solid at ordinary room temperature, but liquid at body temperature, and therefore, melt in the rectum or vaginal cavity and release the active.

Dosage forms for topical administration include ointments, powders, sprays and inhalants. The compound(s) are admixed under sterile conditions with a physiologically acceptable carrier, and any preservatives, buffers, and/or propellants that may be required. Ophthalmic formulations, eye ointments, powders, and solutions are also contemplated as being within the scope of this invention.

Chemical Synthesis

Staring from cytisine, a natural, known compound, numerous compounds were synthesized according to the following methodology.

In general, the first step of a synthesis involving cytisine as starting material is the protection of the amine, using any standard amine protecting group which is stable to further steps used to synthesize the rest of the molecule. One preferred group is a t-butyloxycarbonyl group (t-BOC), although numerous additional blocking groups may be used. From there, individual scaffolds and related substituents on the cytisine molecule are introduced. Most of the modifications are performed using the 2-Pyridone Scaffold or by modifying the chemistry associated with the 2-pyridone scaffold.

For example, bromination is carried out quite readily using the bromination agent N-bromosuccinimide (3-Bromo or 5-Bromo). From the bromo derivative, introduction of numerous groups may be afforded. For example, introduction of aryl and heteroaryl may be afforded using Suzuki coupling reactions under microwave accelerated conditions, in a single mode microwave cavity using power of no more than 30 W for less than 30 minutes, although conventional heating may be used. See, for example, Timari, et al. Synlett 1067-1068 (1997), Larhed, et al. Org Chem 61, 9582-9584 (1996) and Tetrahedron Lett 37, 8219-8222 (1996).

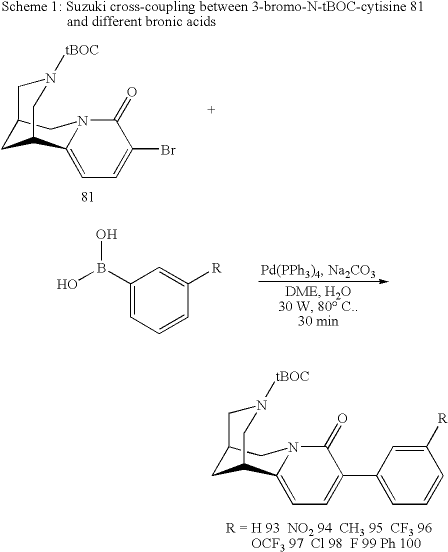

Analogously, 5-substituted (R⁴) alkyl, vinyl and phenyl-substituted cytisine may be similarly synthesized using the Suzuki coupling reaction.

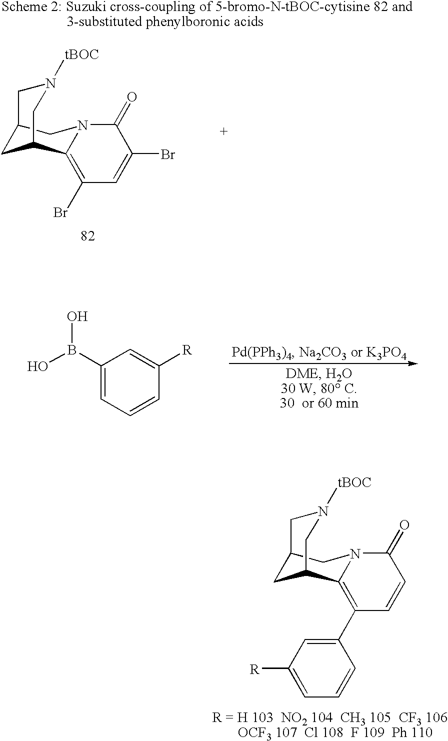

Heterocyclic derivatives of cytisine may be readily synthesized using Suzuki coupling or via a Pd-catalysed Buchwald-Hartwig reaction. See, WO 98/18798, PCT/IB97/01282, U.S. Pat. No. 6,235,734 B1, US 2003/0065173 A1, relevant portions of which are incorporated by reference herein.

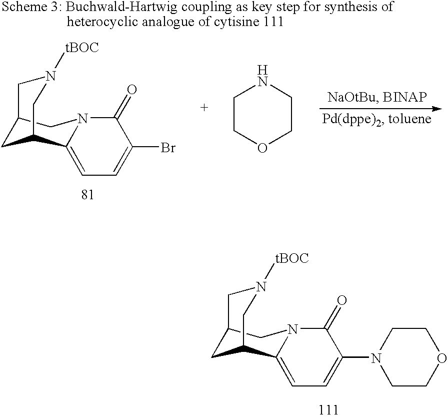

Alternatively, Stille cross-coupling reactions may be used

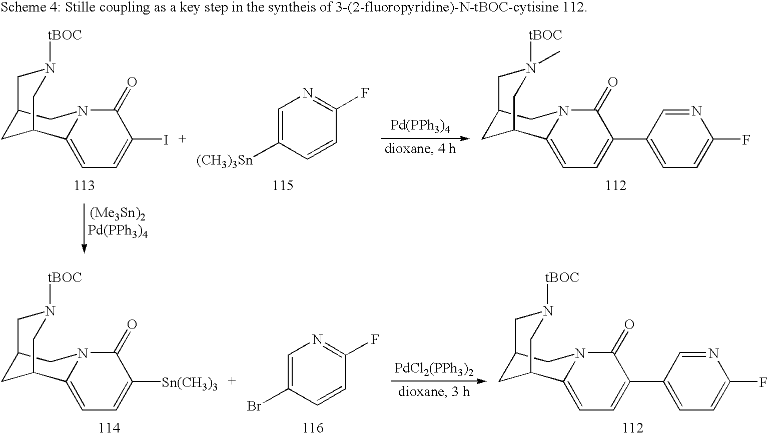

The introduction of heterocyclic scaffolds of biological interest into the position 3 or 5 of the cytisine structure is shown in Schemes 5 and 6, below. While coupling of 5-indolylboronic acid with both 81 or 82 performed well under “standard” condition (entry 117 and 123), the same condition afforded incorporation of 3,4-methylenedioxyphenyl group only into the more reactive position 3 of the pyridone moiety (entry 118). For the same coupling with 5-bromo-N-tBOC-cytisine 82 sodium carbonate had to be replaced by Ba(OH)₂ and the reaction time extended to 60 min (entry 124). The application of K₃PO₄ as a base afforded synthesis of 3-(3′-pyridyl)-N-tBOC-cytisine 119 within 60 min, but the stronger base Ba(OH)₂ had to be employed for the synthesis of the corresponding 5-substituted counterpart (entry 125). The same observation was made for incorporation of 1-methyl-1H-pyrazol-4-yl moiety. Whereas the coupling reaction in the presence of K₃PO₄ as a base gave the desired coupling product 122 in 30 min, the corresponding 5-substituted analogue (entry 126) was synthesized only when Ba(OH)₂ was employed and the reaction time extended to 60 min. Potassium phosphate also allowed incorporation of 4-pyridyl and quinolin-8-yl into the position 3 of the pyridone moiety (entry 120 and 121), however, the introduction of these substituents into position 5 was not successful. All attempts to synthesize 5-(4′-pyridyl)- and 5-(quinolin-8′-yl) analogue failed. The syntheses were carried out under a variety of conditions with regard to the base (Na₂CO₃, K₂CO₃, Cs₂CO₃ or Ba(OH)₂), solvent (DUE or DMF) and reaction time. The microwave heating was stopped after 60 or 90 minutes. However, no combination allowed incorporation of quinolin-8-yl and 4-pyridyl moieties into position 5. For every Suzuki cross-coupling of 81 or 82 with heterocyclic boronic acids, Pd(PPh₃)₄ was employed as a catalyst and DME/H₂O as solvents. Since the unsuccessful syntheses were performed with the same catalyst (Pd(PPh₃)₄), employing different catalyst systems (e.g. PdBnCl(PPh₃)₂ or Pd(OAc)₂/PPh₃) could allow incorporation of 4-pyridyl and quinolin-8-yl moieties into position 5.

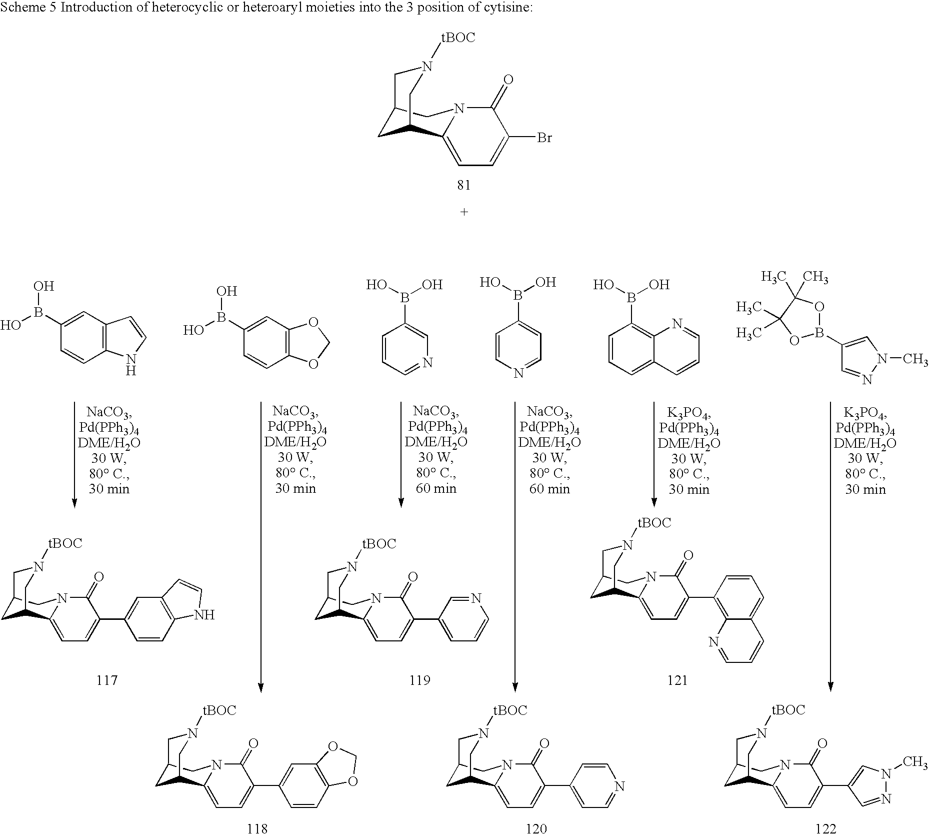

The purification of all N-protected reactions products 117-126 consisted of a solid phase extraction of the highly lipophilic catalyst and a separation of the reaction products from reactants and side products, which was performed with HPLC using isocratic methanol/water mixture.

The coupling reactions with heterocyclic boronic acids confirmed the higher reactivity of position 3. 5-Substituted coupling products were achieved in lower yields and all attempts to introduce quinol-8-yl and 4-pyridyl moieties into the position 5 failed. Similarly, coupling reactions of 3- or 5-bromo-N-tBOC-cytisine 81-82 with 2-chloro-5-pyridylboronic acid yielded the desired coupling product in trace.

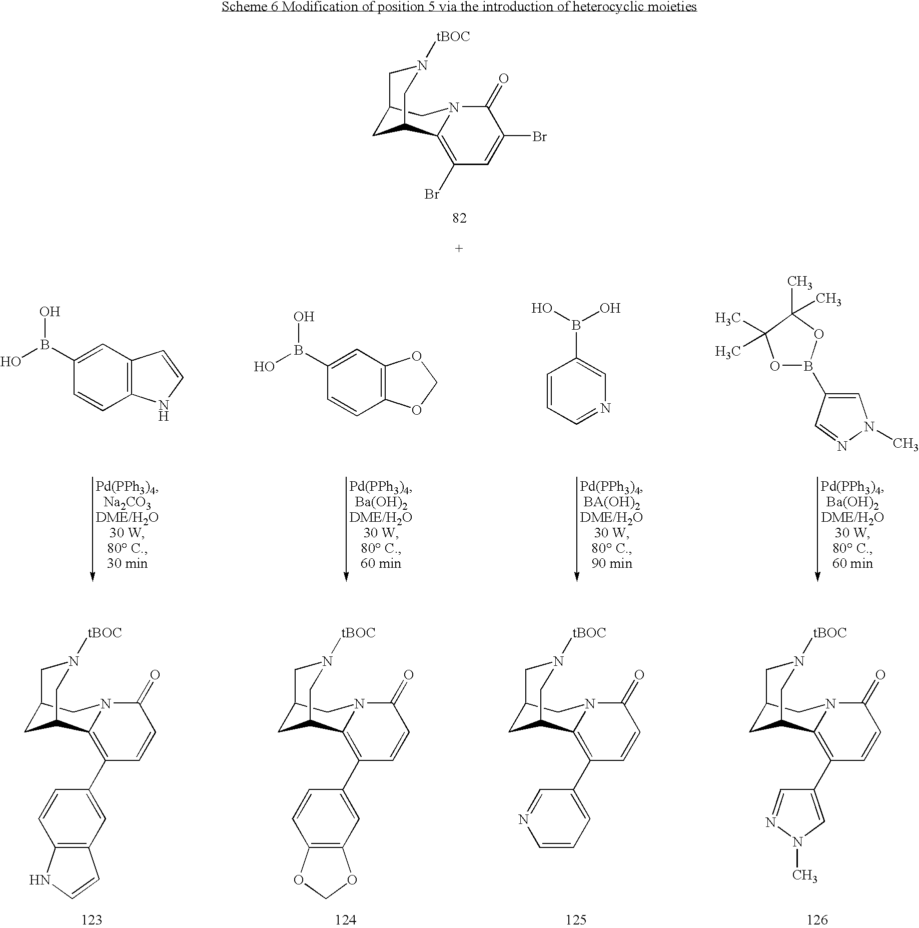

The results of Suzuki cross-coupling with heteroaryl boronic acids extend the original observation from the synthesis of aryl analogues. Microwave activation as an unconventional energy source demonstrated beneficial impact also with the introduction of a heterocyclic unit of pharmacological interest into the structure of cytisine 27. A new strategy for the straightforward and fast synthesis of heterocyclic cytisine analogues yielded ten novel derivatives 117-126 featuring bulky heterocycle in the position 3 or 5.

However, the reaction condition described for phenyl derivatives had to be modified in some protocols, as using the “standard microwave conditions” [Pd(PPh₃)₄, Na₂CO₃, DME/H₂O, 30 W, 30 min], gave only very fair yields. The syntheses of 117-126 were carried out under a variety of conditions regarding the base (Na₂CO₃, K₂CO₃, K₃PO₄, Cs₂CO₃ or Ba(OH)₂), solvent (DME or DMF) and reaction time. Replacement of DME by DMF did not increase yields while the addition of K₃PO₄ or Ba(OH)₂ exerted a remarkable affect on the acceleration of the coupling rate.

The rate and yield enhancing effect of a base is a result of the increasing basic strength of counter anions (HCO₃⁻<MCO₃⁻<MPO₄⁻<OH⁻). Furthermore, counter cations possess different stability constant for halides (Na⁺<K⁺<Cs⁺<Ba²⁺<<Tl⁺<Ag⁺) and for hydroxide anions (Cs⁺<K⁺<Na⁺<Li⁺). Thus, in the example of the synthesis of 3-(4′-pyridyl)-N-tBOC-cytisine 120 (Scheme 7, below), a stronger base K₂PO₄⁻ yields in the transmetallation step a higher concentration of 4-pyridylboronate complex 127 than NaCO₃⁻ does (Scheme 3-31).

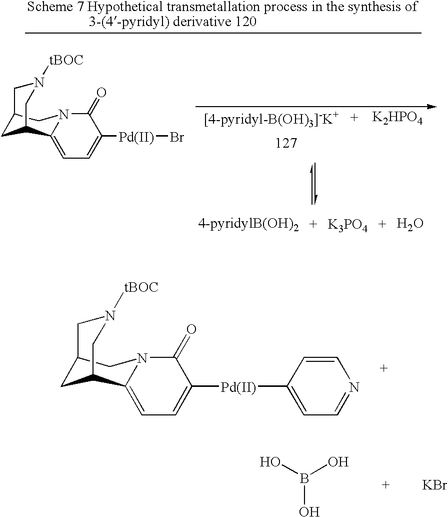

Moreover, K⁺ supports a higher concentration of the boronate 127 because it possesses a lower stability constant for hydroxide anion than Na⁺. On the other hand, potassium cation has a higher stability constant for bromide than Na⁺, meaning that the transmetallation is faster with potassium salts (e.g. K₂CO₃, K₃PO₄) than with sodium salts (e.g. Na₂CO₃).

### 3,5-Disubstituted Analogues of Cytisine

Synthetic routes to disubstituted cytisine analogues 128 and 129 started with 3,5-dibromo-N-tBOC-cytisine 83 (Scheme 8). The Suzuki-cross coupling reaction with phenylboronic acid 74 was selectively performed with the bromine in position 3. When Na₂CO₃ was used as a base, only the bromine in the position 3 of the pyridone moiety coupled with phenylboronic acid. Using 3-pyridylboronic acid and potassium phosphate led to an addition of the 3-pyridyl substituent again only to position 3. For both reactions Pd(PPh₃)₄ was applied as a catalyst and DME/H₂O as solvents. The microwave irradiation of the maximum 30 W was stopped after 30 or 60 min. The purification procedure of the coupling products followed the protocol described for phenyl and heterocyclic derivatives (SPE and HPLC separation).

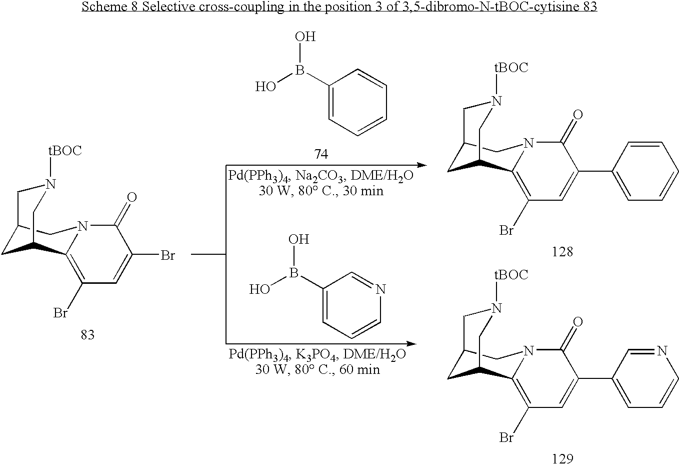

The conditions used in the selective Suzuki cross-coupling reaction were chosen due to previous experience revealing that the more reactive position 3 of the pyridone moiety undergoes coupling with the boronic acid easier and faster. Thus, employing bases such as Na₂CO₃ or K₃PO₄ afforded coupling reaction only in position 3, while the bromine in position 5 was not attacked. In this way obtained 3-phenyl-5-bromo- and 3-(3′-pyridyl)-5-bromo analogues 128 and 129 are novel disubstituted cytisine derivatives.

Removal of the t-BOC group could be afforded using acid or microwave irradiation. Hydrochloric acid quantitatively (reaction time >24 hours) removed the tBOC group and after extraction of a free base into chloroform, final products 93e-100e and 103e-110e were obtained as off-white crystalline powders (Scheme 10, Method A).

However, it was observed that heating the Suzuki reaction with microwave irradiation quantitatively removed the tBOC protecting group. Therefore, after the HPLC purification, the aqueous fraction containing the N-protected product was concentrated to approximately 80 mL and the removal of the carbamate was achieved by thermal fission (150° C.) of CO₂ and isobutene using microwave irradiation of 150 W for 30 minutes (Scheme 10, Method B). As the reaction was quantitative, no purification was required and lyophilization of water for at least 24 h afforded ligands 117e-126e as white or yellowish crystalline powders.

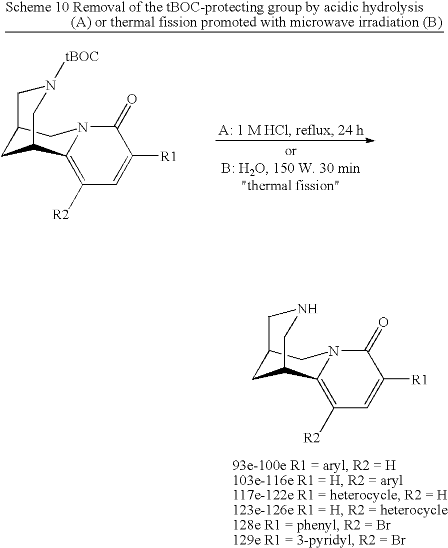

The Synthesis of Novel nAChR Ligands Based Upon Choline

The synthesis of the phenylcarbamate derivatives was carried out according to the Method F (Scheme 11, below). Equimolar amounts of amino alcohols and the appropriate phenylisocyanates were stirred for 1.5-4 hours under argon atmosphere. The reaction was carried out in toluene at 50° C., as reflux temperature was found to reduce yields and increased the formation of the byproducts. The solvent was evaporated and the resulting oily residue was purified by flash chromatography on a small amount of silica gel eluting with dichlormethane/methanol mixture. The products were obtained as yellow oils that crystallised on standing (28%-97%).

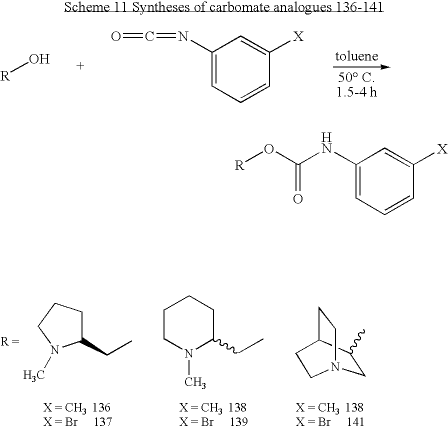

The reaction had to be carried out in a water-free solvent (i.e. dry toluene) under argon atmosphere in order to avoid the decomposition of the isocyanate reactant. Isocyanates are hydrolysed in the presence of water to primary amines and this reaction is catalysed by acids or bases. This may explain why the addition of triethylamine was found to have negative impact on the yields of the syntheses. The primary amine then reacts with isocyanate to give substituted urea derivatives as side-products. Scheme 12 shows an unwanted synthesis of 1,3-di-m-tolyl-urea 142 from m-tolylisocyanate 143 in the presence of water. The side-product 142 (M=240.3 g/mol) crystallised in toluene, thus was easily isolated by filtration and identified via mass spectroscopy ([M+H]⁺=240.2). Interestingly, m-tolylisocyanate always led to the production of side products, whereas this side reaction was not observed when 3-bromopenylisocyanate was employed. Therefore, the yields of methyl analogues 136, 138 and 140 were significantly lower (28%-42%) than the yields of the bromo analogues 137, 139 and 141 (75%-97%).

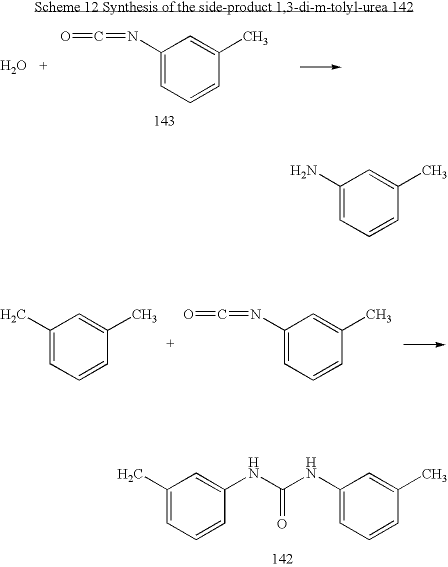

Compounds 144 and 145 were prepared by the Suzuki coupling reaction under microwave irradiation (Scheme 13). Microwave irradiation (60-100 W) and standard Suzuki conditions allowed addition of the phenyl and styryl moieties to the phenyl ring of 141 in less than 10 minutes. The reaction mixture was allowed to cool to room temperature and the solvent evaporated under pressure. The oily residue was purified by column chromatography with the dichloromethane/methanol mixture and crystallised from the mixture of diethyl ether/petroleum ether. The coupling products were obtained as yellow crystalline powders (22%-39%).

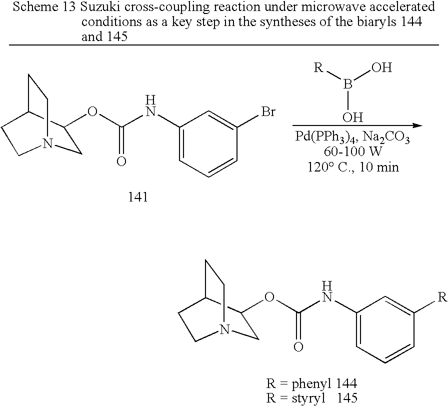

The syntheses of the carbamate derivatives 136-141 were carried out under argon atmosphere and in dry solvents. The addition of triethylamine, which is used for the preparation of carbamate derivatives, did not positively influence the yield of the desired products 136-141. Reflux temperature led to the reduction of yields and increased the production of byproducts, e.g. 142.

The azabicyclic carbamate derivative 141 was used as a reactant for synthesis of the phenyl and styryl analogues 144 and 145. The combination of Suzuki cross-coupling protocol with microwave dielectric heating (previously described) allowed synthesis of the coupling products 144 and 145 in 10 min (22%-39%).

The low yields are results of several impacts and it has to be pointed out that the Suzuki protocol for addition of the phenyl or styryl moieties to the compound 141 was not optimized, since the reactions yielded sufficient amount of the products 144 and 145 for the biological evaluation. Optimization of the experimental protocol for the Suzuki cross-coupling reaction on the cytisine skeleton revealed that the energy of no more than 50 W should be applied in the cross-coupling reactions employing Pd-catalyst (e.g. Pd(PPh₃)₄). Thus, using lower microwave energy could lead to higher yields.

The following examples are presented to exemplify the present invention. These examples are representative in nature and are not to be construed as limiting the invention.

### EXAMPLES

### Methodology

### Ultraturrax Homogenization

The homogenization was completed with ULTRA-TURRAX T50 DPX homogenizator (Janke&Kunkel, IKA Labortechnik, Germany).

### Microwave

Microwave irradiation was carried out using the CEM-Discover microwave synthesis system (CEM GmbH, Kamp-Lintfort, Germany).

### Solid Phase Extraction

SPE was performed on Solid Phase Extraction BAKERBOND Spe™ Columns (KMF Laborchemie Handels GmbH, St. Augustin, Germany).

### High Performance Liquid Chromatography

The chromatographic system consisted of WellChrom Pump K-120 (Knauer GmbH, Germany), Injection & Switching Valves (Knauer GmbH, Germany), Preparative HPLC-Pump K-1800 (Knauer GmbH, Germany), Fast scanning Spectro-Photometer K-2600 (Knauer GmbH, Germany) and Electric Valve Drive (Knauer GmbH, Germany). The column used was Eurospher 100 C 18, 10 μm, 250 mm×20 mm (ID) from Knauer GmbH, Germany. The mobile phase was a mixture of MeOH(HPLC Grade Methanol, Merck KgaA, Darmstadt, Germany) and deionised H₂O. The flow rate of the mobile phase was 20 mL/min and the input of 10 mL was used. The chromatograms were scanned at 254 nm and collected to reaction flasks.

### Lyophilization

Lyophilization of water was carried out using the Alpha 1-4 LSC (Martin Christ Gefriertrocknungsanlagen GmbH, Osterode, Germany).

### Column Chromatography

Column chromatography was carried out on Merck silica gel 60 (70-230 mesh). The solvents were evaporated with Vacuubrand CVC 2 rotary evaporator (Vacuubrand GmbH & Co KG, Wertheim, Germany).

### NMR Spectroscopy

¹H- and ¹³C-NMR spectra (1D and 2D) were measured at 500 MHz and 125 MHz on a Bruker “Avance 500” spectrometer at the Institute for Pharmaceutical Chemistry, Poppelsdorf, University of Bonn. CDCl₃ was used as a solvent and the chemical shift of the remaining protons of the deuterated solvent served as internal standard: δ ¹H 7.24 ppm, δ ¹³C 77 ppm. The assignment was done with the aid of 2D NMR chemical shift maps (COSY, HSQC, HMBC) as well as with the aid of substituent chemical shifts. The coupling constants are given in Hertz (Hz) and the chemical shifts in part per million (ppm). The signal multiplicities are given as follows: s=singlet, d=doublet, t=triplet, q=quartet, sex=sextet, m=multiplet, br=broad, ovl=overlapping, p=pseudo.

### Mass Spectroscopy

The mass spectra (EI With high resolution) were measured on an “MS-50 A.E.I.” or “MAT 95 XL, Thermoquest” at the Kekulé Institute for Organic Chemistry and Biochemistry, Endenich, University of Born

### InfraRed Spectroscopy

Infrared spectra were determined on Perkin-Elmer 1600 Series FTIR (Perkin-Elmer, Wellesley, Mass., USA) spectrophotometer at the institute for Pharmaceutical Chemistry, Poppelsdorf, University of Bonn

### Elemental Microanalysis

Elemental microanalyses were performed on a VarioEL apparatus (Elemetar AnalysenSysteme, GmbH, Hanau, Germany) at the Institute for Pharmaceutical Chemistry, Endenich, University of Bonn.

### Melting Point

Melting points were determined on a Büchi B-545 melting point apparatus and are uncorrected. For some derivatives the melting point was not determined due to the little amount of the product.

### Thin Layer Chromatography

The purity of the compounds was checked on TLC chromatography (Kieselgel 60 F₂₅₄, Merck, Darmstadt, Germany) using CH₂Cl₂/MeOH/EDMA 99:1:1 v/v/v as a mobile phase.

For the reverse phase TLC the RP-18 F₂₅₄s plates (Merck, Darmstadt, Germany) and a mixture MeOH/H₂0 80:20 v/v were utilised.

### Chemical Substances

Commercially obtained chemical substances (purity>97%) were directly used in the chemical reactions. Commercially obtained solvents with the purity<97% were purified via distillation. The boronic acids were obtained from Acros Organics (provided by KMF Laborchemie Handels GmbH, Sankt Augustin, Germany) or Aldrich-Sigma Chemie GmbH, Taufkirchen, Germany in 95%-98% purity.

### Numbering

The alternative numbering used in the thesis for the name and assignment of the ¹H and ¹³C NMR chemical shifts is not consistent with the IUPAC numbering. The IUPAC name of each compound is listed at the end of the spectroscopic characterisation.

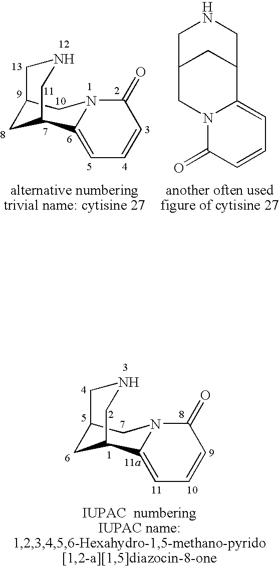

### Intermediates

N-tBOC-cytisine 76, 3-bromo-N-tBOC-cytisine 81, 5-bromo-N-tBOC-cytisine 82 and 3,5-dibromo-N-tBOC-cytisine 83 were synthesized as previously described (See, WO 98/18798, PCT/IB97/01282, U.S. Pat. No. 6,235,734 B1, US 2003/0065173 A1 and were used in following synthetic steps.

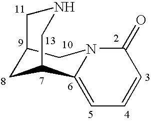

The seeds of Laburnum anagyroides and Laburnum watereri were collected each year in the Köln-Bonn area in the months September-October. The plant material was air-dried at least for 3 months and ground to a powder consistence.

The plant material was extracted with CH₂Cl₂/MeOH/aq.NH₃ through homogenization by Ultra-turrax for 8 hours (Table 6-1). The evaporated solvent were replaced, exactly the same amounts of each solvent were added to the homogenate during the extraction. The homogenate was centrifuged (2,000×min, 40 min) and the supernatant collected. The dark green solution was concentrated under reduced pressure to the final volume of 500 mL and extracted with 1M HCl (3×100 mL). The aqueous acid solution was rendered alkaline with 26% NH₄OH (pH 11-12) and the free base extracted with CH₂Cl₂ (10×100 mL). The organic layers were collected and the solvent evaporated in vacuo. The dark green/brownish residue was chromatographed on silica gel column with CHCl₃/MeOH 6:1 v/v. The alkaloid 27 was recrystallized from perchlorethylene or directly used in the next step (N-tBOC-cytisine 76, Method B).

TABLE 1
Experimental conditions for the extraction of cytisine 27

| Extraction | Amount of the | Solvents* | | | | |
|---|---|---|---|---|---|---|
| No. | plant material | CH₂Cl₂ | MeOH | aq.NH₃ | Cytisine 27 | Yields |
| 1 | 350 g (seeds only) | 500 mL | 140 mL | 60 mL | 0.95 g | 0.27% |
| 2 | 600 g (seeds only) | 840 mL | 240 mL | 90 mL | 2.90 g | 0.48% |
| 3 | 1,000 g | 2.5 L | 1 L | 250 mL | 1.15 g | 0.11% |
| 4 | 1,000 g | 2.5 L | 1 L | 250 mL | 1.83 g | 0.18% |
| 5 | 500 mg | 1.3 L | 500 mL | 125 mL | 0.64 g | 0.13% |
| 6 | 1,000 g | 2.5 L | 1 L | 250 mL | 1.36 g | 0.14% |

*The same amount of solvents added through the 8 hours of homogenization.

M.p.: 155-156° C.

¹H NMR (500 MHz, CDCl₃) δ [ppm] 7.05 (dd, ³J=9.1 Hz, ³J=6.9 Hz, 1H, H4); 6.17 (d, ³J=9.1 Hz, 1H, H3); 5.77 (d, ³J=6.9 Hz, 1H, H5); 3.77 (d, ²J=15.4 Hz, 1H, H10β); 3.57 (dd, ²J=6.6 Hz, ³J=15.7 Hz, 1H, H10α); 2.70-2.75 (m, 4H, H11+H13); 2.62 (s br, 1H, H7); 2.03 (s br, 1H, H9); 1.65 (s br, 2H, H8)

¹³C NMR (125 MHz, CDCl₃) δ [ppm] 162.8 (C═O, C2); 150.7 (C6); 138.1 (C4); 115.7 (C3); 104.2 (C5); 53.3 (C13); 52.3 (C11); 49.1 (C10); 34.9 (C7); 27.0 (C9); 25.6 (C8)

¹H NMR (500 MHz, CD₃OD) δ [ppm] 7.49 (dd, ³J=9.1 Hz, ³J=6.9 Hz, 1H, H4); 6.44 (dd, ³J=9.1 Hz, ⁴J=1.6 Hz, 1H, H3); 6.30 (dd, ³J=6.9 Hz, ⁴J=1.6 Hz, 1H, H5); 4.09 (d, ²J=15.5 Hz, 1H, H10α); 3.92 (dd, ²J=6.6 Hz, ³J=15.5 Hz, 1H, H10β); 2.99-3.11 (m, 5H, H11+H13+H7); 2.39 (s br, 1H, H9); 2.04 (d, ²J=12.9 Hz, 1H, H8_{A}); 1.98 (d, ²J=12.9 Hz 1H, H8_{B})

¹³C NMR (125 MHz, CD₃OD) δ [ppm] 165.8 (C═O, C2); 153.3 (C6); 141.3 (C4); 117.0 (C3); 108.0 (C5); 54.4 (C13); 53.4 (C11); 51.3 (C10); 36.6 (C7); 29.0 (C9); 26.9 (C8)

IUPAC 1,2,3,4,5,6-hexahydro-1,5-methano-pyrido[1,2-a][1,5]diazocin-8-one

### Synthesis of Intermediates

### N-tBOC-cytisine 76

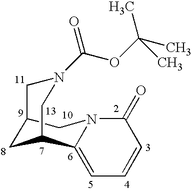

### Method A

Cytisine 27 (500 mg, 2.63 mmol), di-t-butyldicarbonate (688 mg, 3.15 mmol, 1.2 eq) and Na₂CO₃ (334 mg, 3.15 mmol, 1.2 eq) were stirred in 25 mL CH₂Cl₂ and 6 mL H₂O at 60° C. for 2 hours. The reaction mixture was allowed to cool to room temperature and 10 mL of concentrated NaCl solution was added. The organic layer was dried over Mg₂SO₄ and the solvent evaporated. The product was recrystallized from petroleum ether and obtained as off-white crystalline powder (590 mg-690 mg, 77%-90%).

### Method B

The dark brownish oily residue obtained from column chromatography in the isolation step was dissolved in 50 mL of CH₂Cl₂. Di-t-butyldicarbonate (2 g, 9.0 mmol), Na₂CO₃ (954 mg, 9.0 mmol) and 10 mL H₂O were added. The reaction mixture was stirred at 60° C. The reaction was monitored by TLC (CH₂Cl₂/MeOH 9:1 v/v) and reagents (di-t-butyldicarbonate, Na₂CO₃) were added until the cytisine spot disappeared. The mixture was allowed to cool to room temperature, washed with 30 mL of concentrated NaCl solution and the organic layer evaporated under reduced pressure. The yellow residue was dissolved in 150 mL of MeOH/H₂O 60:40 v/v and the product was purified with HPLC.

### HPLC Method

Mobile phase: 0-10′ MeOH/H₂O 60:40 v/v
   10′-15′ gradient to 100% MeOH
   15′-25′ 100% MeOH
Input: 10 mL
Flow rate: 20 mL/min
Run time: 25 min
Detection: UV at λ=254 nm
Retention time: t_{r(N-tBOC-cytisine)}=6.9 min

The collected aqueous layer were concentrated under reduced pressure and dried via lyophilization for at least 24 hours. N-tBOC-cytisine 76 was obtained as white crystalline powder and was directly used in the bromination step. The yields are calculated over the whole isolation/protection procedure and are listed in the Table 6-2.

TABLE 2
Amount of cytisine 27 calculated from the amount of N-tBOC-cytisine 76.
Yields calculated as the ratio of cytisine 27 quantity to the amount of plant
material (1,000 g)

| | Amount of | Solvents* | | | | | |
|---|---|---|---|---|---|---|---|
| | the plant | | | | N-tBOC- | Cytisine | |
| Extraction No. | material | CH₂Cl₂ | MeOH | aq.NH₃ | cytisine 76 | 27 | Yields |
| 7 | 1,000 g | 2.5 L | 1 L | 250 mL | 1.80 g | ~1.18 g | 0.12% |
| 8 | 1,000 g | 2.5 L | 1 L | 250 mL | 1.76 g | ~1.15 g | 0.11% |
| 9 | 1,000 g | 2.5 L | 1 L | 250 mL | 2.70 g | ~1.77 g | 0.17% |
| 10 | 1,000 g | 2.5 L | 1 L | 250 mL | 2.30 g | ~1.51 g | 0.15% |
| 11 | 1,000 g | 2.5 L | 1 L | 250 mL | 2.06 g | ~1.35 g | 0.13% |

*The same amount of solvents added through the 8 hours of homogenization.

¹H NMR (500 MHz, CDCl₃) δ [ppm] 7.24 (dd, ³J=9.1 Hz, ³J=6.3 Hz, 1H, H4); 6.41 (d, ³J=9.1 Hz, 1H, H3); 6.03 (s br, 1H, H5); 4.14 (d, ²J=15.4 Hz, 1H, H10β); 4.00-4.19 (m ovl., 2H, H13); 3.79 (dd, ²J=6.6 Hz, ³J=15.7 Hz, 1H, H10α); 2.94-3.05 (m, 3H, H7+H11); 2.38 (s br, 1H, H9); 1.93 (d, 1H, H8_{A}); 1.87 (d, 1H, H8_{B}); 1.30 (s, 9H, tBOC-group)

¹³C NMR (125 MHz, CDCl₃) δ [ppm] 163.4 (C═O, C2); 154.5 (C═O; tBOC); 148.7 (C6); 138.9 (C4); 117.1 (C3); 105.8 (C5); 80.3 (C(CH₃), tBOC); 51.6 (C13); 50.5 (C11); 48.9 (C10); 34.8 (C7); 28.0 (C(CH₃), tBOC); 27.5 (C9); 26.1 (C8)
IUPAC 8-oxo-1,5,6,8-tetrahydro-2H,4H-1,5-methano-pyrido[1,2-a][1,5]diazocine-3-carboxylic acid tert-butyl ester 3-Bromo-N-tBOC-cytisine 81 and 5-Bromo-N-tBOC-cytisine 82

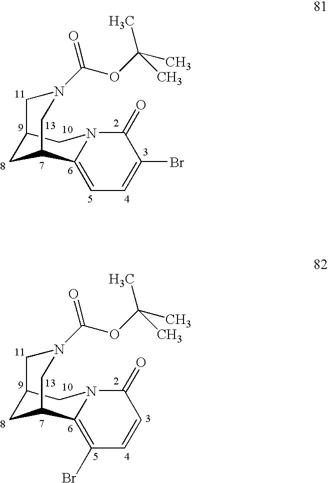

N-tBOC-cytisine 76 (1 g, 3.44 mmol) and N-bromosuccinimide (613 mg, 3.44 mmol, 1 eq) were stirred in 30 mL CH₂Cl₂ at 60° C. for 2 hours. The reaction mixture was allowed to cool to room temperature and the solvent evaporated in vacuo. The oily residue was dissolved in 150 mL of MeOH/H₂O 60:40 v/v and the isomers were separated and purified with HPLC.

### HPLC Method

Mobile phase: MeOH/H₂O 60:40 v/v
Input: 10 mL
Flow rate: 20 mL/min
Run time: 20 min
Detection: UV at λ=254 nm
Retention time: t_{r(3-Br—N-tBOC-cyt)}=8.85 min
   t_{r(5-Br—N-tBOC-cyt)}=11.55 min

The collected aqueous layers were concentrated under reduced pressure and products dried via lyophilization for at least 24 hours. The products were obtained as white crystalline powders in 38.0%-51.9% (81) and 28.1%-42.2% (82) yields (Table 6-3).

TABLE 3
Yields of 3-bromo and 5-bromo-N-tBOC-cytisine isomers 81 and 82

| Experiment | 3-Br—N-tBOC- | | 5-Br—N-tBOC- | |
|---|---|---|---|---|
| No. | cytisine 81 | Yields | cytisine 82 | Yields |
| 1 | 540 mg | 43.0% | 422 mg | 33.6% |
| 2 | 543 mg | 43.2% | 353 mg | 28.1% |
| 3 | 652 mg | 51.9% | 384 mg | 30.6% |
| 4 | 646 mg | 51.4% | 530 mg | 42.2% |
| 5 | 477 mg | 38.0% | 460 mg | 36.6% |
| 6 | 507 mg | 40.4% | 286 mg | 22.8% |
| 7 | 517 mg | 41.2% | 395 mg | 31.5% |
| 8 | 504 mg | 40.1% | 354 mg | 28.2% |

### 3-Bromo-N-tBOC-cytisine 81

¹H NMR (500 MHz, CDCl₃) δ [ppm] 7.64 (d, ³J=7.6 Hz, 1H, H4); 5.96 (s br, 1H, H5); 4.23 (d, ²J=15.8 Hz, 1H, H10; 4.06-4.35 (m ovl., 2H, H13); 3.85 (dd, ²J=6.3 Hz, ³J=15.5 Hz, 1H, H10α); 2.99-3.06 (m, 3H, H7+H11); 2.40 (s br, 1H, H9); 1.94 (t, ²J=13.2 Hz, 2H, H8); 1.30 (s, 9H, tBOC)

¹³C NMR (125 MHz, CDCl₃) δ [ppm] 159.4 (C═O, C2); 154.4 (C═O, tBOC); 148.5 (C6); 140.8 (C4); 112.5 (C3); 105.7 (C5); 80.6 (C(CH₃), tBOC); 51.4 (C13); 50.2 (C11); 49.2 (C10); 34.7 (C7); 28.0 (C(CH₃), tBOC); 27.4 (C9); 26.0 (C8)

IUPAC 9-bromo-8-oxo-1,5,6,8-tetrahydro-2H,4H-1,5-methano-pyrido[1,2-a][1,5]diazocine-3-carboxylic acid tert-butyl ester

### 5-Bromo-N-tBOC-cytisine 82

¹H NMR (500 MHz, CDCl₃) δ [ppm] 7.42 (d, ³J=9.8 Hz, 1H, H4); 6.38 (d, ³J=9.5 Hz, 1H, H3); 4.36 (s br, 2H, H13); 4.16 (d, ²J=15.5 Hz, 1H, H10β); 3.82 (dd, ³J=6.0 Hz, ²J=15.5 Hz, 1H, H10α); 3.42 (s br, 1H, H7); 2.90-3.06 (m, 2H, H11); 2.40 (s br, 1H, H9); 1.97 (s, 2H, H8); 1.29 (s, 9H, tBOC)

¹³C NMR (125 MHz, CDCl₃) δ [ppm] 162.3 (C═O, C2); 154.4 (C═O, tBOC); 145.9 (C6); 142.4 (C4); 118.2 (C3); 99.3 (C5); 80.5 (C(CH₃), tBOC); 50.2 (C13); 48.9 (C11); 47.2 (C10); 34.2 (C7); 28.0 (C(CH₃), tBOC); 27.4 (C9); 26.3 (C8)

IUPAC 11-bromo-8-oxo-1,5,6,8-tetrahydro-2H,4H-1,5-methano-pyrido[1,2-a][1,5]diazocine-3-carboxylic acid tert-butyl ester

### 3,5-Dibromo-N-tBOC-cytisine 83

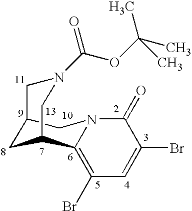

N-tBOC-cytisine 76 (600 mg, 2.0 mmol) and N-bromosuccinimide (700 mg, 4-0 mmol, 2 eq) were stirred in 25 mL CH₂Cl₂ at 60° C. for 2 hours. The reaction mixture was allowed to cool to room temperature and the solvent evaporated in vacuo. The oily residue was dissolved in 100 mL of MeOH/H₂O 60:40 v/v and the product was separated and purified with HPLC.

### HPLC Method

Mobile phase: isocratic MeOH/H₂O 60:40 v/v

Input: 10 mL

Flow rate: 20 mL/min

Run time: 25 min

Detection: UV at λ=254 nm

Retention time: t_{r(3,5-diBr—N-tBOC-cyt)}=16.9 min

The collected aqueous layers were concentrated under reduced pressure and the product dried via lyophilization for at least 24 hours. The product was obtained as white crystalline powder (376 mg, 42%).

¹H NMR (500 MHz, CDCl₃) δ [ppm] 7.83 (s, 1H, H4); 4.33 (s br, 2H, H13); 4.22 (d, ²J=15.8 Hz, 1H, H10β); 3.85 (dd, ³J=7.0 Hz, ²J=16.1 Hz, 1H, H10α); 3.39 (s br, 1H, H7); 2.87-3.10 (m, 2H, H11); 2.39 (s br, 1H, H9); 1.99 (t, ²J=14.1 Hz, 2H, H8), 1.29 (s, 9H, tBOC)

¹³C NMR (125 MHz, CDCl₃) δ [ppm] 158.6 (C═O, C2); 154.2 (C═O, tBOC); 145.5 (C6); 143.8 (C4); 113.2 (C3); 98.3 (C5); 80.7 (C(CH₃), tBOC); 51.4 (C13); 48.9 (C11); 47.9 (C10); 34.2 (C7); 28.0 (C(CH₃), tBOC); 27.3 (C9); 26.2 (C8)

IUPAC 9,11-dibromo-8-oxo-1,5,6,8-tetrahydro-2H,4H-1,5-methano-pyrido[1,2-a][1,5]diazocine-3-carboxylic acid tert-butyl ester

Synthesis of Novel nAChR Ligands Based on Cytisine

General Procedures

### Suzuki Cross-Coupling Reaction

The appropriate bromo-N-tBOC-cytisine isomer (81-83, 0.27 mmol), boronic acid or ester (0.41 mmol; 1.5 eq), base (0.6 mmol, 2.2 eq), DME (3 mL) or DMF (3 mL) and H₂O (1 mL) were placed in a 10-mL microwave glass tube. The solution was washed with argon for 10 min. After the addition of Pd(PPh₃)₄ (0.027 mmol, 0.1 eq) the reaction vessel was sealed with a septum and placed into the microwave cavity. Microwave irradiation of 30 W was used, the temperature being ramped from room temperature to 80° C. Once 80° C. was reached, the reaction mixture was held for 30 or 60 min. Then the mixture was allowed to cool to room temperature, the reaction vessel was opened and the solvent evaporated under pressure. The brown residue was extracted on SPE C-18 column eluting with mixture MeOH/H₂O 70:30 or 60:40 v/v. The aqueous solution was concentrated in vacuo and the tBOC-protected product was purified by HPLC.

### HPLC Method

The ratios of MeOH/H₂O 80:20 (v/v), 70:30 (v/v), 60:40 (v/v), 55:45 (v/v), 50:50 (v/v) were used for 25 min. In the following 5 min gradient to 100% MeOH was run and the system was washed with MeOH for 15 min. The flow rate of the mobile phase was 20 mL/min and the input of 10 mL was used. The chromatograms were scanned at 254 nm and collected to reaction flasks. The retention time given for each compound is the retention time of the N-tBOC-protected analogue. The fraction containing the desired product was concentrated in vacuo using a rotary evaporator.

### Deprotection—Method A

To the concentrated aqueous solution (approximately 15 mL) of the tBOC-protected product was added 1M HCl (15 mL) and the mixture was stirred at reflux for 24 hours. The reaction mixture was allowed to cool to room temperature and NaHCO₃ was added. Free base was extracted with CHCl₃, the organic solvent was evaporated and the desired product dried in vacuo.

### Deprotection—Method B

The concentrated aqueous solution of the tBOC-protected product (approximately 70 mL) was put into a 80-mL microwave glass tube, sealed and placed into a microwave cavity. Microwave irradiation of 150 W was used, the temperature being ramped from room temperature to 150° C. Once 150° C. was reached, the reaction mixture was held for 30 min. Then the mixture was allowed to cool to room temperature, the reaction vessel was opened and the solvent evaporated by lyophilization for at least 24 hours.

### 3-Phenyl-cytisine 93e

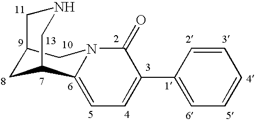

The Suzuki reaction was performed according to the general method with 3-bromo-N-tBOC-cytisine 81 (100 mg, 0.27 mmol), phenylboronic acid (50 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification, a mixture of MeOH/H₂O 70:30 v/v (100 mL) was used. The HPLC separation was completed with MeOH/H₂O 60:40 v/v. Deprotection by Method B. The final product obtained as white crystalline powder (42 mg, 0.15 mmol, 58.4%).

M.p.: 139.8-140.6° C.

HPLC: tᵣ=16.73 min

¹H NMR (500 MHz, CDCl₃) δ [ppm] 7.69 (dt, ⁴J=1.3 Hz, ³J=7.2 Hz, 2H, H2′+H6′); 7.46 (d, ³J=7.2 Hz, 1H, H4), 7.38 (t, ³J=7.2 Hz, 2H, H3′+H5′); 7.29 (tt, ⁴J=1.3 Hz, ³J=7.2 Hz, 1H, H4′), 6.09 (d, ³J=7.2 Hz, 1H, H5); 4.19 (d, ²J=15.5 Hz, 1H, H10β); 1H, H10β); 3.96 (dd, ²J=6.3 Hz, ³J=15.5 HZ, 1H, H10α); 3.03 (d, ²J=12.3 Hz, 1H, H11_{A}); 3.07 (dd, ³J=2.2 Hz, ²J=12.3 Hz, 1H, H13_{A}); 3.02 (d br, J=12.3 Hz, 2H, H11_{B}+H13_{B}); 2.91 (s br, 1H, H7); 2.34 (s br, 1H, H9); 1.96 (s br, 2H, H8)

¹³C NMR (125 MHz, CDCl₃) δ [ppm] 162.1 (C═O, C2); 150.3 (C6); 137.4 (C4); 137.0 (C1′); 128.6 (C2′+C6′); 128.0 (C3′+C5′); 127.4 (C3); 127.2 (C4′); 105.0 (C5); 54.0 (C13); 53.0 (C11); 50.2 (C10); 35.7 (C7); 27.9 (C9); 26.3 (C8)

MS (EI) m/z 266.2 (100), 223.1 (65), 210.1 (20), 185.1 (25), 167.1 (10), 149.1 (10), 133.1 (10), 115.1 (10), 97.1 (10)

| | | |
|---|---|---|
| HRMS for C₁₇H₁₈N₂O | calc. | 266.1419 |
| | found | 266.1426 |

IUPAC 9-phenyl-1,2,3,4,5,6-hexahydro-1,5-methano-pyridino[1,2-a]diazocin-8-one

### 3-(3′-Nitro-phenyl)-cytisine 94e

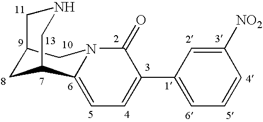

The Suzuki reaction was performed according to the general method with 3-bromo-N-tBOC-cytisine 81 (100 mg, 0.27 mmol), 3-nitrophenylboronic acid (68 mg, 0.41 mmol), Na₂CO₃ (64 mg, 0.6 mmol), Pd(PPh₃)₄ (30 mg, 0.027 mol), DME and H₂O. The reaction time was 30 min. For the SPE purification, a mixture of MeOH/H₂O 70:30 v/v (100 mL) was used. The HPLC separation was completed with MeOH/H₂O 60:40 v/v. Deprotection by Method A. The final product obtained as yellow crystalline powder (70 mg, 0.22 mmol, 83%).

M.p.: 209.3-209.8° C.

HPLC: tᵣ=17.18 min

¹H NMR (500 MHz, CDCl₃) δ [ppm] 8.53 (t, ⁴J=1.9 Hz, 1H, H2′); 8.10-8.13 (m, ovl., 2H, H4′+H6′); 7.54 (d, ³J=7.3 Hz, 1H, H4); 7.52 (t, ³J=8.0 Hz, 1H, H5′); 6.13 (d, ³J=7.3 Hz, 1H, H5); 4.17 (d, ²J=15.7 Hz, 1H, H10β); 3.95 (dd, ³J=6.6 Hz, ²J=15.7 Hz, 1H, H10α); 2.98-3.14 (m, 4H, H11+H13); 2.95 (s br, 1H, H7); 2.38 (s br, 1H, H9); 1.97 (s, 2H, H8); 1.87 (s br, NH)

¹³C NMR (125 MHz, CDCl₃) δ [ppm] 161.7 (C═O); 152.0 (C6); 148.2 (C3′); 139.0 (C1′); 137.6 (C4); 134.7 (C6′); 128.9 (C5′); 124.7 (C3); 123.3 (C2′); 121.9 (C4′); 105.0 (C5); 53.8 (C13); 52.9 (C11); 50.3 (C10); 35.7 (C7); 27.7 (C9); 26.1 (C8)

MS (EI) m/z 311.2 (90), 268.1 (100), 255.1 (20), 230.1 (15), 82.1 (25), 44.0 (10)

| | | |
|---|---|---|
| HRMS for C₁₇H₁₇N₃O₃ | calc. | 311.1269 |
| | found | 311.1267 |

IUPAC 9-(3-nitro-phenyl)-1,2,3,4,5,6-hexahydro-1,5-methano-pyridino[1,2-a]diazocin-8-one

### 3-(3′-Methyl-phenyl)-cytisine 95e

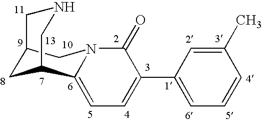

The Suzuki reaction was performed according to the general method with 3-bromo-N-tBOC-cytisine 81 (100 mg, 0.27 mmol), m-tolylboronic acid (55 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification, a mixture of MeOH/H₂O 70:30 v/v (100 mL) was used. The HPLC separation was completed with MeOH/H₂O 65:35 v/v. Deprotection by Method A. The final product obtained as off-white crystalline powder (49 mg, 0.16 mmol, 65%).

M.p.: n.d.

HPLC: tᵣ=16.27 min

¹H NMR (500 MHz, CDCl₃) δ [ppm] 7.52 (s br, 1H, H2′); 7.43 (d, ³J=7.3 Hz, 2H, H4+H6′); 7.26 (t, ³J=7.3 Hz, 1H, H5′); 7.09 (d, ³J=7.3 Hz, 1H, H4′); 6.06 (d, ³J=7.0 Hz, 1H, H5); 4.18 (d, ²J=15.6 Hz, 1H, H10β); 3.94 (dd, ³J=6.6 Hz, ²J=15.6 Hz, 1H, H10α), 2.91 (s br, 1H, H7); 3.00-3.11 (m, 4H, H11+H13); 2.35 (s, 3H, CH₃); 2.34 (s, 1, H9); 1.97 (s, 2H, H8)

¹³C NMR (125 MHz, CDCl₃) δ [ppm] 162.2 (C═O); 150.0 (C6); 137.5 (C1′); 137.3 (C3′); 137.0 (C4); 129-3 (C2′); 128.0 (C4′); 128.0 (C5′); 127.6 (C3); 125.7 (C6′); 105.0 (C5); 53.9 (C13); 52.8 (C11); 50.1 (C10); 35.6 (C7); 27.8 (C9); 26.3 (C8); 21.5 (CH₃)

MS (EI) m/z 280.2 (80), 237.1 (100), 224.1 (25), 199.1 (20), 82.1 (10), 44.0 (40)

| | | |
|---|---|---|
| HRMS for C₁₈H₂₀N₂O | calc. | 280.1576 |
| | found | 280.1579 |

IUPAC 9-m-tolyl-1,2,3,4,5,6-hexahydro-1,5-methano-pyridino[1,2-a]diazocin-8-one

### 3-(3′-Trifluoromethyl-phenyl)-cytisine 96e

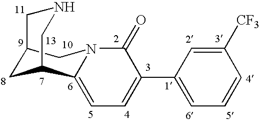

The Suzuki reaction was performed according to the general method with 3-bromo-N-tBOC-cytisine 81 (100 mg, 0.27 mmol), 3-trifluoromethylphenylboronic acid (77 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification, a mixture of MeOH/H₂O 70:30 v/v (100 mL) was used. The HPLC separation was completed with MeOH/H₂O 65:35 v/v. Deprotection by Method A. The final product obtained as off-white crystalline powder (47 mg, 0.14 mmol, 52%).

M.p.: 140.9-143.0° C.

HPLC: tᵣ=19.28 min

¹H NMR (500 MHz, CDCl₃) δ [ppm] 7.95 (s, 1H, H2′); 7.91 (d, ³J=7.8 Hz, 1H, H6′); 7.52 (d, ³J=7.8 Hz, 1H, H4′); 7.49 (d, ³J=7.0 Hz, 1H, H4); 7.47 (t, ³J=7.8 Hz, 1H, H5′); 6.11 (d, ³J=7.0 Hz, 1H, H5); 4.16 (d, ²J=15.6 Hz, 1H, H10β); 3.95 (dd, ³J=6.3 Hz, ²J=15.6 Hz, 1H, H10α); 2.99-3.13 (m, 4H, H11+H13); 2.94 (s br, 1H, H7); 2.35 (s br, 1H, H9); 1.96 (s, 2H, H8)

¹³C NMR (125 MHz, CDCl₃) δ [ppm] 161.9 (C═O, C2); 151.4 (C6); 138.1 (C1′); 137.4 (C4); 131.9 (C6′); 130.4 (q, ²J_{C,F}=31.7 Hz, C3′); 128.4 (C5′); 125.8 (C3); 125.3 (q, ³J_{C,F}=3.7 Hz, C4′); 123.8 (q, ³J_{C,F}=3.7 Hz, C2′); 123.2 (q, ¹J_{C,F}=272.5 Hz, CF₃), 104.9 (C5); 54.0 (C13); 53.0 (C11); 50.3 (C10); 35.7 (C7); 27.8 (C9); 26.2 (C8)

MS (EI) m/z 334.2 (85), 291.1 (100), 270.0 (25), 253.1 (35), 82.1 (30), 44.0 (90)

| | | |
|---|---|---|
| HRMS for C₁₈H₁₇F₃N₂O | calc. | 334.1293 |
| | found | 334.1294 |

IUPAC 9-(3-trifluoromethyl-phenyl)-1,2,3,4,5,6-hexahydro-1,5-methano-pyridino[1,2-a]-diazocin-8-one

### 3-(3′-Trifluoromethoxy-phenyl)-cytisine 97e

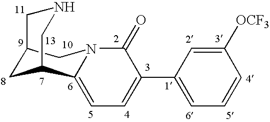

The Suzuki reaction was performed according to the general method with 3-bromo-N-tBOC-cytisine 81 (100 mg, 0.27 mmol), 3-trifluoromethoxyphenylboronic acid (62 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification, a mixture of MeOH/H₂O 70:30 v/v (100 mL) was used. The HPLC separation was completed with MeOH/H₂O 65:35 v/v. Deprotection by Method A. The final product obtained as off-white crystalline powder (61 mg, 0.17 mmol, 64%).

M.p.: n.d.

HPLC: tᵣ=21.28 min

¹H NMR (500 MHz, CDCl₃) δ [ppm] 7.63 (ddd, ⁴J=1.3 Hz, ⁴J=1.6 Hz ³J=8.0 Hz, 1H H6′); 7.58 (s br, 1H, H2′); 7.45 (d, ³J=7.3 Hz, 1H, H4); 7.38 (t, ³J=8.0 Hz, 1H, H5′); 7.11 (dquint, ⁴J=1.3 Hz, ³J=8.0Hz, 1H, H4′); 6.07 (d, ³J=7.3 Hz, 1H, H5); 4.17 (d, ²J=15.5 Hz, 1H, H10β); 3.93 (dd, ³J=6.6 Hz, ²J=15.5 Hz, 1H, H10α); 2.95-3.12 (m, 4H, H11+H13); 2.91 (s br, 1H, H7); 2.35 (s br, 1H, H9); 1.95 (s br, 2H, H8)

¹³C NMR (125 MHz, CDCl₃) δ [ppm] 161.8 (C═O, C2); 151.1 (C6); 149.0 (q, ³J_{C,F}=1.5 Hz, C3′); 139.3 (C1′); 137.3 (C4); 129.2 (C5′); 126.9 (C6′); 125.6 (C3); 121.5 (q, ¹J_{C,F}=257.0 Hz, CF₃); 121.1 (C4′); 119.5 (C2′); 105.0 (C5); 53.7 (C13); 52.8 (C11); 50.2 (C10); 35.6 (C7); 27.7 (C9); 26.1 (C8)

MS (EI) m/z 350.2 (100), 307.1 (100), 294.1 (20), 269.1 (30), 82.0 (20), 44.0 (50)

| | | |
|---|---|---|
| HRMS for C₁₈H₁₇F₃N₂O₂ | calc. | 350.1242 |
| | found | 350.1244 |

IUPAC 9-(3-trifluoromethoxy-phenyl)-1,2,3,4,5,6-hexahydro-1,5-methano-pyridino[1,2-a]diazocin-8-one

### 3-(3′-Chloro-phenyl)-cytisine 98e

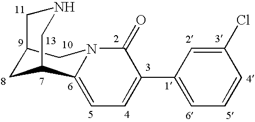

The Suzuki reaction was performed according to the general method with 3-bromo-N-tBOC-cytisine 81 (100 mg, 0.27 mmol), 3-chlorophenylboronic acid (63 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification, a mixture of MeOH/H₂O 70:30 v/v (100 mL) was used. The HPLC separation was completed with MeOH/H₂O 65:45 v/v. Deprotection by Method B. The final product obtained as white crystalline powder (38 mg, 0.13 mmol, 47%).

M.p.: 190.6-190.9° C.

HPLC: tᵣ=20.39 min

¹H NMR (500 MHz, CDCl₃) δ [ppm] 7.69 (t, ⁴J=1.6 Hz, 1H, H2′); 7.58 (dt, ⁴J=1.6 Hz, ³J=7.9 Hz, 1H, H4′); 7.44 (d, ³J=7.31 Hz, 1H, H4); 7.29 (t, ³J=7.9 Hz, 1H, H5′); 7.23 (dd, ⁴J=1.6 Hz, ³J=7.9 Hz, 1H, H6′); 6.08 (d, ³J=7.3 Hz, 1H, H5); 4.16 (d, ²J=15.6 Hz, 1H, H10β); 3.94 (dd, ³J=6.0 Hz, ²J=15.6 Hz, 1H, H10α); 3.11 (d, ²J=12.0 Hz, 1H, H11_{A}); 3.06 (dd, ³J=2.2 HZ, ²J=12.0 Hz, 1H, H13_{A}); 3.00 (d, ²J=12.0 Hz, 2H, H11_{B}+H13_{B}); 2.92 (s br, 1H, H7); 2.35 (s br, 1H, H9); 1.96 (s br, 2H, H8)

¹³C NMR (125 MHz, CDCl₃) δ [ppm] 161.8 (C═O, C2); 151.0 (C6); 139.1 (C1′); 137.3 (C4); 133.9 (C3′); 129.2 (C5′); 128.6 (C2′); 127.2 (C4′); 126.7 (C6′); 125.9 (C3); 105.0 (C5); 53.9 (C13); 53.0 (C11); 50.2 (C10); 35.7 (C7); 27.8 (C9); 26.2 (C8)

MS (EI) m/z 300.1 (100), 257.1 (95), 244.0 (30), 219.0 (40), 192.1 (5), 150.0 (10), 82.1 (10), 68.1 (5)

| | | |
|---|---|---|
| HRMS for C₁₇H₁₇ClN₂O | calc. | 300.1029 |
| | found | 300.1025 |

IUPAC 9-(3-chlorophenyl)-1,2,3,4,5,6-hexahydro-1,5-methano-pyridino[1,2-a]diazocin-8-one

### 3-(3′-Fluoro-phenyl)-cytisine 99e

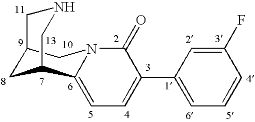

The Suzuki reaction was performed according to the general method with 3-bromo-N-tBOC-cytisine 81 (100 mg, 0.27 mmol), 3-fluorophenylboronic acid (56 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification, a mixture of MeOH/H₂O 70:30 v/v (100 mL) was used. The HPLC separation was completed with MeOH/H₂O 65:35 v/v. Deprotection by Method A. The final product obtained as off-white crystalline powder (35 mg, 0.12 mmol, 46%).

M.p.: 138.9-140.0° C.

HPLC: tᵣ=13.7 min

¹H NMR (500 MHz, CDCl₃) δ [ppm] 7.45 (d, ³J=7.2 Hz, 1H, H4); 7.43 (d br, ³J=8.2 Hz, 2H, H2′+H6′); 7.32 (dt, ⁴J=6.3 Hz, ³J=8.5 Hz, 1H, H5′); 6.96 (tdd, ⁴J=0.9 Hz, ⁴J=2.5 Hz, ³J=8.5 Hz, 1H, H4′); 6.08 (d, ³J=7.2 Hz, 1H, H5); 4.17 (d, ²J=15.6 Hz, 1H, H10β); 3.93 (dd, ³J=6.0 Hz, ²J=15.6 Hz, 1H, H10α); 3.12 (d, ²J=12.0 Hz, 1H, H11_{A}); 3.06 (dd, ³J=2.2 Hz, ²J=12.0 Hz, 1H, H13_{A}); 3.01 (d, ²J=12.0 Hz, 1H, H11_{B}); 2.98 (d, ²J=13.0 Hz, 1H, H13_{B}); 2.92 (s br, 1H, 7); 2.36 (s br, H, H9); 2.26 (s br, NH); 1.96 (s br, 2H, H8)

¹³C NMR (125 MHz, CDCl₃) δ [ppm] 163.6 (d, ¹J_{C,F}=244.1 Hz, C3′); 161.9 (C═O, C2); 150.7 (C6); 139.4 (d, ³J_{C,F}=8.2 Hz, C1′); 137.3 (C4); 129.4 (d, ³J_{C,F}=8.5 Hz, C5′); 126.0 (d, ⁴J_{C,F}=2.3 Hz, C3); 124.1 (d, ⁴J_{C,F}=2.7 Hz, C6′); 115.6 (d, ²J_{C,F}=22.4 Hz, C4′); 114.1 (d, ²J_{C,F}=21.2 Hz, C2′); 105.0 (C5); 53.6 (C13); 52.7 (C11); 50.1 (C10); 35.5 (C7); 27.7 (C9); 26.1 (C8)

MS (EI) m/z 284.2 (100), 241.2 (100), 228.1 (30), 203.1 (40), 149.1 (25), 82.1 (20), 44.0 (50)

| | | |
|---|---|---|
| HRMS for C₁₇H₁₇FN₂O | calc. | 284.1325 |
| | found | 284.1324 |

IUPAC 9-(3-fluoro-phenyl)-1,2,3,4,5,6-hexahydro-1,5-methano-pyridino[1,2-a]diazocin-8-one

### 3-(Biphenyl-3′-yl)-cytisine 100e

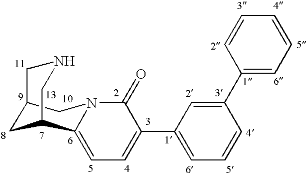

The Suzuki reaction was performed according to the general method with 3-bromo-tBOC-cytisine 81 (100 mg, 0.27 mmol), 3-biphenylboronic acid (80 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification mixture of MeOH/H₂O 80:20 v/v (100 mL) was used. The HPLC separation was completed with MeOH/H₂O 65:35 v/v for 15 min, then a gradient to the final mixture of MeOH/H₂O 80:20 v/v was run for 15 min. Deprotection by Method A. The final product obtained as off-white crystalline powder (33 mg, 0.1 mmol, 36%).

M.p.: 95.2-97° C.

HPLC: tᵣ=27.29 min

¹H NMR (500 MHz, CDCl₃) δ [ppm] 7.91 (t, ⁴J=1.6 Hz, 1H, H2′); 7.69 (ddd, ⁴J=1.3 Hz, ⁴J=1.6 Hz, ³J=8.0 Hz, 1H, H6′), 7.62 (dt, ⁴J=1.3 Hz, ³J=8.2Hz, 2H, H2″+H6″); 7.51 (d, ³J=7.3 Hz, 1H, H4; dd, ovl., 1H, H4′); 7.44 (d, ³J=8.0 Hz, 1H, H5′); 7.40 (tt, ⁴J=1.9 Hz, ³J=7.0 Hz, 2H, H3″+H5″); 7.31 (tt, ⁴J=1.3 Hz, ³J=7.0 Hz, 1H, H4′); 6.09 (d, ³J=7.3 Hz, 1H, H5); 4.19 (d, ²J=15.6 Hz, 1H, H10β); 3.97 (dd, ³J=5.7 Hz, ²J=15.6 Hz, 1H, H10α); 3.00-3.13 (m, 4H, H11+H13); 2.93 (s br, 1H, H7); 2.35 (s br, 1H, H9); 1.97 (s br, 2H, H8)

¹³C NMR (125 MHz, CDCl₃) δ [ppm] 162.1 (C═O, C2), 150.4 (C6), 141.4 (C3′), 141.0 (C1″), 137.6 (C4), 137.8 (C1′), 129.1 (C3), 128.5*, 128.1*, 127.8*, 127.4*, 127.1*, 126.8*, 126.6*, 125.5*, 105.3 (C5), 53.9 (C13), 53.0 (C11), 50.2 (C10), 35.3 (C7), 27.5 (C9), 26.3 (C8)

MS (EI) m/z 342.2 (100), 299.2 (65), 262.1 (60), 183.1 (35), 108.0 (10)

| | | |
|---|---|---|
| HRMS for C₂₃H₂₂N₂O | calc. | 342.1732 |
| | found | 342.1734 |

IUPAC 9-biphenyl-3-yl-1,2,3,4,5,6-hexahydro-1,5-methano-pyridino[1,2-a]diazocin-8-one

### 5-Phenyl-cytisine 103e

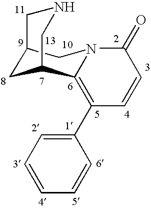

The Suzuki reaction was performed according to the general method with 5-bromo-N-tBOC-cytisine 82 (100 mg, 0.27 mmol), phenylboronic acid (50 mg, 0.41 mmol), Na₂CO₃ (64 mg, 0.6 mmol), Pd(PPh₃)₄ (30 mg, 0.027 mmol), DME and H₂O. The reaction time was 60 ml. For the SPE purification, a mixture of MeOH/H₂O 70:30 v/v (100 mL) was used. The HPLC separation was completed with MeOH/H₂O 60:40 v/v. Deprotection by Method B. The final product obtained as white crystalline powder (28 mg, 0.1 mmol, 39%).

M.p.: 91° C.

HPLC: tᵣ=17.32 min

¹H NMR (500 MHz, CDCl₃) δ [ppm] 7.37 (tt, ⁴J=1.3 Hz, ³J=7.3, 2H, H3′+H5′); 7.31 (tt, ⁴J=1.6 Hz, ³J=7.3 Hz, 1H, H4′); 7.21 (d, ³J=9.5 Hz, 1H, H4); 7.18 (tt, ⁴J=1.6 Hz, ³J=7.3 Hz, 2H, H2′+H6′); 6.49 (d, ³J=9.5 Hz, 1H, H3), 4.19 (d, ²J=15.6 Hz, 1H, H10β); 3.95 (dd, ³J=6.9 Hz, ²J=15.6 Hz; 1H, H10α); 3.12 (d, ²J=12.0 Hz, 1H, H11_{A}); 3.04 (s br, 1H, H7); 2.92 (d, ²J=12.0 Hz, 1H, H11_{B}); 2.81 (d, ²J=12.0 Hz, 1H, H13_{A}); 2.69 (dd, ³J=2.5 Hz, ²J=12.0 Hz, 1H, H13_{B}), 2.30 (s br, 1H, H9); 1.92 (d, ²J=13.0 Hz, 1H, H8_{A}); 1.84 (d, ²J=13.0 Hz, 1H, H8_{B})

¹³C NMR (125 MHz, CDCl₃) δ [ppm] 163.1 (C═O); 147.5 (C6); 141.4 (C4); 138.5 (C1′); 129.8 (C2′+C6′); 128.6 (C3′+C5′); 127.4 (C4′); 119.3 (C5); 116.1 (C3); 53.0 (C13); 52.2 (C11); 50.4 (C10); 31.6 (C7); 27.4 (C9); 26.4 (C8)

MS (EI) m/z 266.2 (100), 223.1 (100), 210.1 (30), 185.1 (20), 167.1 (10), 149.1 (10), 133.1 (10), 82.1 (10)

| | | |
|---|---|---|
| HRMS for C₁₇H₁₈N₂O | calc. | 266.1419 |
| | found | 266.1416 |

IUPAC 11-phenyl-1,2,3,4,5,6-hexahydro-1,5-methano-pyridino[1,2-a]diazocin-8-one

### 5-(3′-Nitro-phenyl)-cytisine 104e

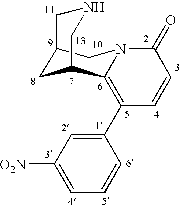

The Suzuki reaction was performed according to the general method with 5-bromo-N-tBOC-cytisine 82 (100 mg, 0.27 mmol), 3-nitrophenylboronic acid (68 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification, a mixture of MeOH/H₂O 70:30 v/v (100 mL) was used. The HPLC separation was completed with MeOH/H₂O 60:40 v/v. Deprotection by Method A. The final product obtained as yellow crystalline powder (52 mg, 0.17 mmol, 62%).

M.p.: 175.6-177.2° C.

HPLC: tᵣ=16.7 min

¹H NMR (500 MHz, CDCl₃) δ [ppm] 8.19-8.21 (m, 1H, H4′); 8.09-8.11 (m, 1H, H2′); 7.55-7.60 (m, 2H, H5′+H6′); 7.18 (d, ³J=9.3 Hz, 1H, H4); 6.52 (d, ³J=9.3 Hz, 1H, H3); 4.19 (d, ²J=15.5 Hz, 1H, H10β); 3.96 (dd, ²J=6.6 Hz, ³J=15.5 Hz, 1H, H10α); 3.10 (d, ²J=9.1 Hz, 1H, H11_{A}); 2.93 (s br, 2H, H7+H11_{B}); 2.74 (d br, ²J=9.1 Hz, 2H, H13); 2.35 (s br, 1H, H9); 1.94 (s br, 1H, H8_{A}); 1.87 (s br, 1H, H8_{B})

¹³C NMR (125 MHz, CDCl₃) δ [ppm] 163.0 (C═O, C2); 148.4 (C3′); 148.1 (C6); 141.0 (C4); 140.0 (C1′); 135.5 (C6′); 129.2 (C5′); 124.2 (C2′); 121.9 (C4′); 117.2 (C5); 116.3 (C3); 52.9 (C13); 52.0 (C11); 50.5 (C10); 32.1 (C7); 27.7 (C9); 26.3 (C8)

MS (EI) m/z 311.2 (65), 268.1 (100), 255.1 (20), 230.1 (15), 183.1 (10), 167.1 (10), 149.1 (10), 82.1 (15), 68.1 (10)

| | | |
|---|---|---|
| HRMS for C₁₇H₁₇N₃O₃ | calc. | 311.1270 |
| | found | 311.1269 |

IUPAC 11-(3-nitro-phenyl)-1,2,3,4,5,6-hexahydro-1,5-methano-pyridino[1,2-a]diazocin-8-one

### 5-(3′-Methyl-phenyl)-cytisine 105e

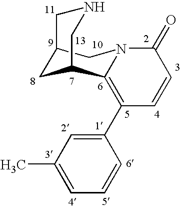

The Suzuki reaction was performed according to the general method with 5-bromo-N-tBOC-cytisine 82 (100 mg, 0.27 mmol), m-tolylboronic acid (55 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification, a mixture of MeOH/H₂O 70:30 v/v (100 mL) was used. The HPLC separation was completed with MeOH/H₂O 65:35 v/v. Deprotection by Method A. The final product obtained as off-white crystalline powder (44 mg, 0.16 mmol, 58%).

M.p.: n.d.

HPLC: tᵣ=16.17 min

¹H NMR (500 MHz, CDCl₃) δ [ppm] 7.25 (t, ³J=7.5 Hz, 1H, H5′); 7.19 (d, ³J=92 Hz, 1H, H4); 7.12 (d br, ³J=7.5 Hz, 1H, H4′); 6.99 (d br, ³J=7.5 Hz, 1H, H6′; s br, ovl., 1H, H2′); 6.47 (d, ³J=9.2 Hz, 1H, H3); 4.19 (d, ²J=15.5 Hz, 1H, H10β); 3.93 (ddd, ⁴J=0.9 Hz, ³J=6.9 Hz, ²J=15.5 Hz, 1H, H10α); 3.07 (s br, 1H, H7); 3.04 (d, ²J=12.3 Hz, H11_{A}); 2.91 (d, ²J=12.3 Hz, 1H, H11_{B}); 2.81 (d, ²J=12.3 Hz, 1H, H13_{A}); 2.68 (dd, ³J=2.5 Hz, ²J=12.3 Hz, 1H, H13_{B}); 2.35 (s, 3H, CH₃); 2.29 (s br, 1H, H9); 1.92 (d br, ²J=13.0 Hz, 1H, H8_{A}); 1.83 (d br, ²J=13.0 Hz, 1H, H8_{B})

¹³C NMR (125 MHz, CDCl₃) δ [ppm] 163.1 (C═O, C2); 147.4 (C6); 141.4 (C4); 138.4 (C1′); 138.3 (C3′); 130.4 (C2′); 128.4 (C5′); 128.1 (C4′); 126.8 (C6′); 119.5 (C5); 116.0 (C3); 53.0 (C13); 52.2 (C11); 50.4 (C10); 31.6 (C7); 27.4 (C9); 26.4 (C8); 21.4 (CH₃)

MS (EI) m/z 280.2 (80), 237.1 (100), 224.1 (25), 199.1 (20), 82.1 (10), 44.0 (30)

| | | |
|---|---|---|
| HRMS for C₁₈H₂₀N₂O | calc. | 280.1576 |
| | found | 280.1577 |

IUPAC 11-m-tolyl-1,2,3,4,5,6-hexahydro-1,5-methano-pyridino[1,2-a]diazocin-8-one

### 5-(3′-Trifluoromethyl-phenyl)-cytisine 106e

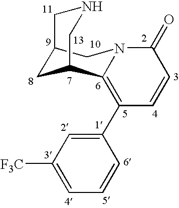

The Suzuki reaction was performed according to the general method with 5-bromo-N-tBOC-cytisine 82 (100 mg, 0.27 mmol), 3-trifluoromethylphenylboronic acid (77 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification mixture of MeOH/H₂O 70:30 v/v (100 mL) was used. The HPLC separation was completed with MeOH/H₂O 65:35 v/v. Deprotection by Method A. The final product obtained as off-white crystalline powder (58 mg, 0.17 mmol, 64%).

M.p.: 155.2-157.0° C.

HPLC: tᵣ=17.05 min

¹H NMR (500 MHz, CDCl₃) δ [ppm] 7.58 (d, ³J=7.9 Hz, 1H, H6′); 7.50 (t, ³J=7.9 Hz, 1H, H5′); 7.46 (s, 1H, H2′); 7.39 (d, ³J=7.9 Hz, 1H, H4′); 7.17 (d, ³J=9.2 Hz, 1H, H4), 6.49 (d, ³J=9.2 Hz, 1H, H3); 4.18 (d, ²J=15.7 Hz, 1H, H10β); 3.94 (dd, ³J=6.0 Hz, ²J=15.7 Hz, 1H, H10α); 3.06 (d, ²J=12.3 Hz, 1H, H11_{A}); 2.93 (s br, 1H, H7); 2.90 (d, ²J=12.3 Hz, 1H, H11_{B}); 2.75 (d, ²J=12.3 Hz, 1H, H13_{A}); 2.69 (dd, ³J=2.5 Hz, ²J=12.3 Hz, 1H, H13_{B}); 2.31 (s br, 1H, H9); 1.93 (d br, ²J=12.9 Hz, 1H, H8_{A}), 1.82 (d br, ²J=12.9 Hz, 1H, H8_{B})

¹³C NMR (125 Mhz, CDCl₃) δ [ppm] 163.0 (C═O, C2); 148.0 (C6); 140.8 (C4); 139.3 (C1′); 133.2 (C6′); 130.9 (q, ²J_{C,F}=32.2 Hz, C3′); 129.2 (C5′); 126.5 (q, ³J_{C,F}=3.7 Hz, C2′); 124.9 (q, ¹J_{C,F}=272.5 Hz, CF₃), 124.3 (q, ³J_{C,F}=3.7 Hz; C4′); 117.6 (C5); 116.4 (C3); 53.0 (C13); 52.1 (C11); 50.5 (C10); 31.7 (C7); 27.3 (C9); 26.3 (C8)

MS (EI) m/z 334.2 (80), 291.1 (100), 253.1 (20), 196.1 (5), 183.1 (5), 167.1 (10), 149.1 (10), 97.1 (5), 82.1 (10)

| | | |
|---|---|---|
| HRMS for C₁₈H₁₇F₃N₂O | calc. | 334.1293 |
| | found | 334.1295 |

IUPAC 11-(3-trifluoromethyl-phenyl)-1,2,3,4,5,6-hexahydro-1,5-methano-pyridino[1,2-a]diazocin-8-one

### 5-(3′-Trifluoromethoxy-phenyl)-cytisine 107e

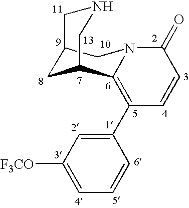

The Suzuki reaction was performed according to the general method with 5-bromo-tBOC-cytisine 82 (100 mg, 0-27 mmol), 3-trifluoromethoxyphenylboronic acid (62 mg, 0.41 mmol), K₃PO₄ (60 mg, 0.6 mmol), Pd(PPh₃)₄ (30 mg, 0.027 mmol), DME and H₂O. The reaction time was 60 min. For the SPE purification, a mixture of MeOH/H₂O 70:30 v/v (100 mL) was used. The HPLC separation was done with MeOH/H₂O 65:35 v/v. Deprotection by Method B. The final product obtained as off-white crystalline powder (26 mg, 0.07 mmol, 27%).

M.p.: n.d.

HPLC: tᵣ=21.12 min

¹H NMR (500 MHz, CDCl₃) δ [ppm] 7.41 (t, ³J=8.0 Hz, 1H, H5′); 7.20 (ddd, ⁴J=1.3 Hz, ⁴J=2-5 Hz, ³J=8.0 Hz, 1H, H4′); 7.19 (d, ³J=9.2 Hz, 1H, H4); 7.15 (dt, ⁴J=1.3 Hz, ³J=8.0 Hz, 1H, H6′); 7.08 (d, ⁴J=2.5 Hz, 1H, H2′); 6.50 (d, ³J=9.2 Hz, 1H, H5); 4.26 (d, ²J=15.7 Hz, 1H, H10β); 3.95 (dd, ³J=6.9 Hz, ²J=15.7H, 1H, H10α); 3.39 (d, ²J=12.3 Hz, 1H, H11_{A}); 3.11 (s br, 1H, H7); 3.07 (d, ²J=12.3 Hz, 1H, H11_{B}); 2.92 (d, ²J=12.3 Hz, 1H, H13_{A}); 2.82 (dd, ³J=2.5 Hz, ²J=12.3 Hz, 1H, H13_{B}); 2.46 (s br, 1H, H9); 1.91 (s br, 2H, H8)

¹³C NMR (125 MHz, CDCl₃) δ [ppm] 163.2 (C═O, C2), 149.3 (q, ³J=1.5 Hz, C3′), 148.3 (C6), 140.0 (C1′), 141.1 (C4), 130.2 (C5′), 128.2 (C6′), 122.3 (C4′), 120.0 (C2′), 119.4 (q, J=257.6 Hz, CF₃), 117.0 (C3), 116.4 (C5), 51.2 (C13), 50.4 (C11), 49.8 (C10), 30.5 (C7), 26.4 (C9), 25.5 (C8)

MS (EI) m/z 350.2 (60), 307.1 (100), 294.1 (25), 269.1 (20), 82.0 (30), 44.0 (50)

| | | |
|---|---|---|
| HRMS for C₁₈H₁₇F₃N₂O₂ | calc. | 350.1242 |
| | found | 350.1252 |

IUPAC 11-(3-trifluoromethoxy-phenyl)-1,2,3,4,5,6-hexahydro-1,5-methano-pyridino[1,2-a]diazocin-8-one

### 5-(3′-Chloro-phenyl)-cytisine 108e

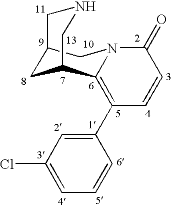

The Suzuki reaction was performed according to the general method with 5-bromo-N-tBOC-cytisine 82 (100 mg, 0.27 mmol), 3-chlorophenylboronic acid (63 mg, 0.41 mmol), K₃PO₄ (60 mg, 0.6 mmol), Pd(PPh₃)₄ (30 mg, 0.027 mmol), DME and H₂O. The reaction time was 60 min. For the SPE purification, a mixture of MeOH/H₂O 70:30 v/v (100 mL) was used. The HPLC separation was done with MeOH/H₂O 65:45 v/v. Additional flash chromatography on silica gel done with CH₂Cl₂/MeOH/EDMA 99:1:1 v/v/v and the purification was completed with HPLC (MeOH/H₂O 65.35 v/v for 25 min). Deprotection by Method B. The final product obtained as white crystalline powder (18 mg, 0.06 mmol, 22%).

M.p.: 80.6-81° C.

HPLC: 20.52 min

¹H NMR (500 MHz, CDCl₃) δ [ppm] 7.32 (m, ovl., 2H, H2′+H4′); 7.21 (t, ³J=8.3 Hz, 1H, H5′); 7.17 (d, ³J=9.2 Hz, 1H, H4); 7.09 (dd, ⁴J=1.6 Hz; ³J=8.3 Hz, 1H, H6′); 6.49 (d, ³J=9.2 Hz, 1H, H3); 4.21 (d, ²J=15.6 Hz, 1H, H10β); 3.95 (dd, ³J=6.1 Hz, ²J=15.6 Hz, 1H, H10α); 3.17 (d, ²J=12.6 Hz, 1H, H11_{A}); 3.04 (s br, 1H, H7); 2.95 (d, ²J=12.6 Hz, 1H, H13_{A}); 2.81 (dd, ³J=2.2 Hz, ²J=12.6 Hz, 1H, H13_{B}); 2.73 (d, ²J=12.6 Hz, 1H, H11_{B}); 2.34 (s br, 1H, H9); 1.93 (s br, 2H, H8)

¹³C NMR (125 MHz, CDCl₃) not available due to the small amount of the sample

MS (EI) m/z 300.1 (100), 257.1 (95), 244.0 (30), 219.0 (40), 192.1 (5), 150.0 (10), 82.1 (10), 68.1 (5)

| | | |
|---|---|---|
| HRMS for C₁₇H₁₇ClN₂O | calc. | 300.1029 |
| | found | 300.1025 |

IUPAC 11-(3-chlorophenyl)-1,2,3,4,5,6-hexahydro-1,5-methano-pyridino[1,2-a]diazocin-8-one

### 5-(3′-Fluoro-phenyl)-cytisine 109e

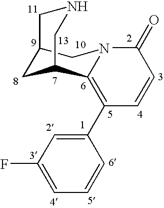

The Suzuki reaction was performed according to the general method with 5-bromo-N-tBOC-cytisine 82 (100 mg, 0.27 mmol), 3-fluorophenylboronic acid (56 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification, a mixture of MeOH/H₂O 70:30 v/v (100 mL) was used. HPLC separation was completed with MeOH/H₂O 65:35 v/v. Deprotection by Method A. The final product obtained as off-white crystalline powder (62 mg, 0.22 mmol, 81%).

M.p.: n.d.

HPLC: tᵣ=12.32 min

¹H NMR (500 MHz, CDCl₃) δ [ppm] 7.32 (td, ⁴J=6.0 Hz, ³J=7.9 Hz, 1H, H15′); 7.16 (d, ³J=9.2 Hz, 1H, H4); 7.01 (tdd, ⁴J=0.9 Hz, ⁴J=2.5 Hz, ³J=8.5 Hz, 1H, H4′); 6.96 (dt, ⁴J=1.4 Hz, ³J=7.9 Hz, 1H, H6′); 6.90 (dt, ⁴J=1.4 Hz, ³J=7.9 Hz, 1H, H2′); 6.47 (d, ³J=9.2 Hz, 1H, H3); 4.17 (d, ²J=15.7 Hz, 1H, H10β); 3.92 (dd, ³J=6.8 Hz, ²J=15.7 Hz, 1H, H10α); 3.03 (d, ²J=11.3 Hz, 1H, H11_{A}); 3.07 (s br, 1H, H7), 2.93 (s br, 1H, H11_{B}); 2.79 (d br, ²J=11.0 Hz, 1H, H13_{B}); 2.71 (d br, ²J=11.0 Hz, 1H, H13_{B}); 2.31 (s br, 1H, H9); 1.91 (s br, 2H, H8_{A}+NH); 1.84 (d br, ²J=12.6 Hz, 1H, H8_{B})

¹³C NMR (125 MHz, CDCl₃) δ [ppm] 163.6 (¹J_{C,F}=247.6 Hz; C3′); 163.0 (C═O, C2); 147.6 (C6); 140.9 (C4); 140.6 (³J_{C,F}=7.7 Hz, C1′); 130.1 (⁴J_{C,F}=8.5 Hz, C5′); 125.6 (⁴J_{C,F}=2.8 Hz, C6′); 118.0 (²J_{C,F}=1.7 Hz, C5); 116.8 (²J_{C,F}=21.0 Hz, C2′); 116.2 (C3), 114.4 (²J_{C,F}=21.0 Hz, C4′); 52.8 (C13); 52.1 (C11); 50.4 (C10), 31.6 (C7); 27.3 (C9); 26.2 (C8)

MS (EI) m/z 284.1 (95), 241.0 (100), 228.0 (30), 203.0 (20), 149.0 (20), 133.0 (15), 82.1 (10), 57.1 (10)

| | | |
|---|---|---|
| HRMS for C₁₇H₁₇FN₂O | calc. | 284.1325 |
| | found | 284.1334 |

IUPAC 11-(3-fluoro-phenyl)-1,2,3,4,5,6-hexahydro-1,5-methano-pyridino[1,2-a]diazocin-8-one

### 5-(Biphenyl-3′-yl)-cytisine 110e

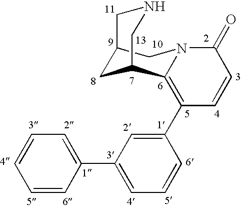

The Suzuki reaction was performed according to the general method with 5-bromo-N-tBOC-cytisine 82 (100 mg, 0.27 mmol), 3-biphenylboronic acid (80 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification, a mixture of MeOH/H₂O 80:20 v/v (100 mL) was used. The HPLC separation was done with MeOH/H₂O 65:35 v/v for 15 min, subsequently a gradient to the final mixture of MeOH/H₂O 80:20 v/v was nm for 15 min. Deprotection by Method A. The final product obtained as off-white crystalline powder (58 mg, 0.17 mmol, 62%).

M.p.: 168.5-169.8° C.

HPLC: tᵣ=27.65 min

¹H NMR (500 MHz, CDCl₃) δ [ppm] 7.55-7.58 (m, 3H, H2′+H2″+H6″); 7.41-7.46 (m, 4H, H3″+H5″+H5′+H6′); 7.35 (tt, ⁴J=1.6 Hz, ³J=7.6 Hz, 1H, H4″); 7.26 (d, ³J=9.1 Hz, 1H, H4); 7.17 (dt, ⁴J=1.6 Hz, ³J=7.6 Hz, 1H, H4′); 6.51 (d, ³J=9.1 Hz, 1H, H3); 4.22 (d, ²J=15.4 Hz, 1H, H10β); 3.96 (dd, ³J=6.6 Hz, ²J=15.4 Hz, 1H, H10α); 3.09 (s, 1H, H7); 3.07 (d, ²J=12.3 Hz, 1H, H11_{A}); 2.91 (d ²J=123 Hz, 1H, H11_{B}); 2.85 (d, ²J=12.3 Hz, 1H, H13_{A}); 2.72 (dd, ³J=2.5 Hz, ²J=1.23 Hz, 1H, H13_{B}), 2.30 (s, 1H, H9); 1.94 (d br, ²J=12.9 Hz, 1H, H8_{A}; 1.84 (d br, ²J=12.9 Hz, 1H, H8_{B}); 1.23 (br s, 1H, NH)

¹³C NMR (125 MHz, CDCl₃) δ [ppm] 163.1 (C═O); 147.7 (C6); 141.6 (C1″); 141.3 (C4); 140.6 (C3′); 139.0 (C1′); 129.1*; 128.6*; 128.9*; 128.6*; 128.5*; 127.1*; 127.6*; 126.1*; 119.1 (C5); 116.1 (C3); 53.0 (C13); 52.3 (C11); 50.5 (C10); 31.4 (C7); 27.4 (C9); 26.4 (C8) * the assignment of the biphenyl moiety could not be completed

MS (EI) m/z 342.2 (100), 299.2 (90), 262.1 (50), 201.20 (20), 183.1 (30), 170.1 (20), 149.0 (20)

| | | |
|---|---|---|
| HRMS for C₂₃H₂₂N₂O | calc. | 342.1732 |
| | found | 342.1734 |

IUPAC 11-biphenyl-3-yl-1,2,3,4,5,6-hexahydro-1,5-methano-pyridino[1,2-a]diazocin-8-one

### 3-(1H-Indol-5′-yl)-cytisine 117e

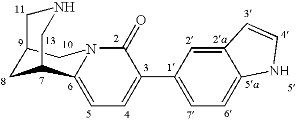

The Suzuki reaction was performed according to the general method with 3-bromo-N-tBOC-cytisine 81 (100 mg, 0.27 mmol), 5-indolylboronic acid (65 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification, a mixture of MeOH/H₂O 60:40 v/v (100 mL) was used. The HPLC separation was completed with MeOH/H₂O 60:40 v/v. Deprotection by Method B. The final product obtained as yellowish crystalline powder (31 mg, 0.1 mmol, 37%).

M.p.: 139.0-141.1° C.

HPLC: tᵣ=16.12 min

¹H NMR (500 MHz, CDCl₃) δ [ppm] 8.28 (s br, 1H, indolic NH); 7.92 (s br, 1H, H2′); 7.52 (dd, ⁴J=1.6 Hz, ³J=8.5 Hz, 1H, H7′); 7.47 (d, ³J=7.0 Hz, 1H, H4); 7.38 (d, ³J=8.5 Hz, 1H, H6′); 7.16 pseudo t, ³J=2.8 Hz, 1H, H4′); 6.54 (tt, ⁴J=0.9 Hz, ³J=22 Hz, 1H, H3′); 6.06 (d, ³J=7.0 Hz, 1H, H5); 4.22 (d, ²J=15.6 Hz, 1H, H10β); 3.96 (dd, ³J=6.3 Hz, ²J=15.6 Hz, 1H, H10α); 3.12 (d, ²J=12.6 Hz, 1H, H11_{A}); 3.00-3.06 (m, 3H, H11_{B}+H13); 2.89 (s br, 1H, H7); 2.34 (s br, 1H, H9); 1.96 (s br, 2H, H8)

¹³C NMR (125 MHz, CDCl₃) δ [ppm] 162.6 (C═O); 148.9 (C6); 136.7 (C4); 135.3 (C5a); 129.1 (C1′); 128.9 (C3); 127.8 (C2a); 124.3 (C4′); 123.2 (C7′); 120.9 (C2′); 110.5 (C6′); 105.1 (C5); 103.0 (C3′); 53.8 (C13); 52.9 (C11); 50.1 (C10); 35.6 (C7); 27.9 (C9); 26.4 (C8)

MS (EI) m/z 305.2 (100), 261.1 (70), 249.2 (20), 233.2 (20), 44.0 (10)

| | | |
|---|---|---|
| HRMS for C₁₉H₁₉N₃O | calc. | 305.1528 |
| | found | 305.1531 |

IUPAC 9-(1H-Indol-5-yl)-1,2,3,4,5,6-hexahydro-1,5-methano-pyridino[1,2-a]diazocin-8-one

### 3-(3′,4′-Methylenedioxy-phenyl)-cytisine 118e

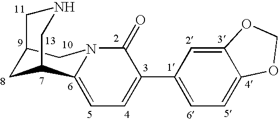

The Suzuki reaction was performed according to the general method with 3-bromo-N-tBOC-cytisine 81 (100 mg, 0.27 mmol), 3,4-methylenedioxyphenylboronic acid (68 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification, a mixture of MeOH/H₂O 70:30 v/v (100 mL) was used. The HPLC separation was completed with MeOH/H₂O 60:40 v/v. Deprotection by Method B. The final product obtained as off-white crystalline powder (30 mg, 0.1 μmmol, 36%).

M.p.: 259.1-261.6° C.

HPLC: tᵣ=18.99 min

¹H NMR (500 MHz, CDCl₃), δ [ppm] 7.38 (d, ³J=7.3 Hz, 1H, H4); 7.26 (d, ⁴J=1.6 Hz, 1H, H2′); 7.12 (dd, ⁴J=1.6 Hz, ³J=8.2, 1H, H6′); 6.81 (cl, ³J=8.2 Hz, 1H, H5′); 6.05 (d, ³J=7.3 Hz, 1H, H5); 5.94 (s, 2H, CH₂); 4.16 (d, ²J=15.7 Hz, 1H, H10β); 3.93 (dd, ³J=6.6 Hz, ²J=15.7 Hz, 1H, H10α); 3.11 (d, ²J=12.0 Hz, 1H, H11_{A}); 3.06 (dd, ³J=2.2 Hz, ²J=12.0 Hz, 1H, H13_{A}); 3.00 (d br; ²J=12.0 Hz, 2H, H11_{B}+H13_{B}); 2.90 (s br, 1H, H7); 2.34 (s br, 1H, H9); 1.95 (s br, 2H, H8)

¹³C NMR (125 MHz, CDCl₃) δ [ppm] 162.1 (C═O, C2); 149.9 (C6); 147.3 (C3′); 146.8 (C4′); 136.4 (C4); 131.4 (C1); 127.1 (C3); 122.1 (C6′); 109.4 (C2′); 108.0 (C5); 104.9 (C5); 100.9 (CH₂); 54.0 (C13); 53.0 (C11); 50.2 (C10); 35.7 (C7); 27.9 (C9); 26.4 (C8)

MS (EI) m/z 310.1 (100), 267.0 (70), 254.0 (20), 229.0 (25), 180.0 (5), 155.0 (10), 140.0 (10)

| | | |
|---|---|---|
| HRMS for C₁₈H₁₈N₂O₃ | calc. | 310.1317 |
| | found | 310.1324 |

IUPAC 9-(benzo[1,3]dioxol-5-yl)-1,2,3,4,5,6-hexahydro-1,5-methano-pyridino[1,2-a]diazocin-8-one

### 3-(Pyridin-3′-yl)-cytisine 119e

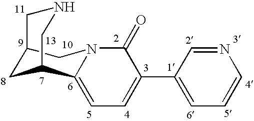

The Suzuki reaction was performed according to the general method with 3-bromo-N-tBOC-cytisine 81 (100 mg, 0.27 mmol), 3-pyridineboronic acid (49 mg, 0.41 mmol), K₃PO₄ (126 mg, 0.6 mmol), Pd(PPh₃)₄ (30 mg, 0.027 mmol), DME and H₂O. The reaction time was 60 nm. For the SPE purification, a mixture of MeOH/H₂O 60:40 v/v (100 mL) was used. The HPLC separation was completed with MeOH/H₂O 50:50 v/v. Deprotection by Method B. The final product obtained as yellow crystalline powder (48 mg, 0.18 mmol, 66%).

M.p.: 79.8-81.6° C.

HPLC: tᵣ=16.05 min

¹H NMR (500 MHz, CDCl₃) δ [ppm] 8.78 (d, ⁴J=1.6 Hz, 1H, H2′); 8.49 (dd, ⁴J=1.6 Hz, ³J=4.7 Hz, 1H, H4′); 8.16 (ddd, ⁴J=1.6H, ⁴J=2.2 Hz, ³J=7.9 Hz, 1H, H6′); 7.49 (d, ³J=7.3 Hz, 1H, H4); 7.29 (ddd, ⁵J=0.9 Hz, ³J=4.7 Hz, ³J=7.9 Hz, 1H, H5′); 6.11 (d, ³J=7.3 Hz, 1H, H5); 4.16 (d, ²J=15.6 Hz, 1H, H11β); 3.94 (dd, ³J=6.9 Hz, ²J=15.6 Hz, 1H, H10α); 3.11 (d, ²J=12.0 Hz, 1H, H11_{A}); 3.06 (dd, ³J=2.2 Hz, ²J=12.0 Hz, 1H, H13_{A}); 2.99-3.03 (m, 2H, H11_{B}+H13_{B}); 2.95 (s br, 1H, H7); 2.36 (s br, 1H, H9); 1.96 (s br, 2H, H8)

¹³C NMR (125 MHz, CDCl₃) δ [ppm] 161.9 (C═O, C2); 151.5 (C6); 149.1 (C2′); 148.2 (C4′); 137.2 (C4); 136.1 (C6′); 133.2 (C1′); 123.9 (C3); 122.8 (C5′); 105.0 (C5); 53.9 (C13); 53.0 (C11); 50.3 (C10); 35.7 (C7); 27.8 (C9); 26.2 (C8)

MS (EI) m/z 267.1 (100), 223.1 (100), 211.1 (20), 186.1 (25), 82.1 (5)

| | | |
|---|---|---|
| HRMS for C₁₆H₁₇N₃O | calc. | 267.1371 |
| | found | 267.1376 |

IUPAC 9-pyridin-3-yl-1,2,3,4,5,6-hexahydro-1,5-methano-pyridino[1,2-a]diazocin-8-one

### 3-(Pyridin-4′-yl)-cytisine 120e

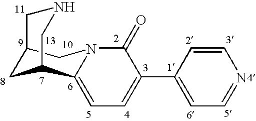

The Suzuki reaction was performed according to the general method with 3-bromo-N-tBOC-cytisine 81 (100 mg, 0.27 mmol), 4-pyridineboronic acid (49 mg, 0.41 mmol), K₃PO₄ (126 mg, 0.6 mmol), Pd(PPh₃)₄ (30 mg, 0.027 mmol), DME and H₂O. The reaction time was 90 min. For the SPE purification, a mixture of MeOH/H₂O 60:40 v/v (100 mL) was used. The HPLC separation was completed with MeOH/H₂O 50:50 v/v. Deprotection by Method B. The final product obtained as yellow crystalline powder (45 mg, 0.17 mmol, 62%).

M.p.: n d.

HPLC: tᵣ=17.8 min

¹H NMR (500 MHz, CDCl₃) δ [ppm] 8.57 (dd, ⁴J=1.6 Hz, ³J=6.1 Hz, 2H, H3′+H5′); 7.67 (dd, ⁴J=1.6 Hz, ³J=6.1 Hz, 2H, H2′+H6′); 7.57 (d, ³J=7.4, 1H, H4); 6.13 (d, ³J=7.4 Hz, 1H, H5); 4.16 (d, ²J=15.8 Hz, 1H, H10β); 3.95 (dd, ³J=6.6 Hz, ²J=15.8 Hz, 1H, H10α); 3.11 (dd, ³J=2.5 Hz, ²J=12.3 Hz, 1H, H11_{A}); 3.07 (dd, ³J=2.5 Hz, ²J=12.3 Hz, 1H, H13_{A}); 3.04 (d, ²J=12.3 Hz, 1H, H13_{B}); 3.00 (ddd, ⁴J=1.2 Hz, ³J=2.5 Hz, ²J=12.3 Hz, 1H, H11_{B}); 2.94 (s br, 1H, H7), 236 (s br, 1H, H9), 1.96 (s br, 2H, H8)

¹³C NMR (125 MHz, CDCl₃) δ [ppm] 161.5 (C═O, C2); 152.6 (C6); 149.6 (C3′+C5′); 144.9 (C1′); 137.8 (C4); 123.9 (C2′+C6′); 122.8 (C3); 104.9 (C5); 53.9 (C13), 53.0 (C11), 50.3 (C10), 35.8 (C7), 27.8 (C9), 26.2 (C8)

MS (EI) m/z 267.1 (100), 223.1 (100), 211.1 (25), 186.1 (40), 149.1 (10), 133.6 (10), 117.1 (10), 82.1 (10)

| | | |
|---|---|---|
| HRMS for C₁₆H₁₇N₃O | calc. | 267.1371 |
| | found | 267.1379 |

IUPAC 9-pyridinyl-1,2,3,4,5,6-hexahydro-1,5-methano-pyridino[1,2-a]diazocin-8-one

### 3-Quinolin-8′-yl-cytisine 121e

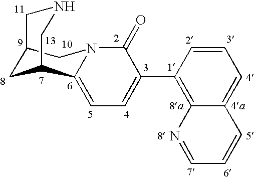

The Suzuki reaction was performed according to the general method with 3-bromo-N-tBOC-cytisine 81 (50 mg, 0.13 mmol), 8-quinolineboronic acid (35 mg, 0.2 mmol), K₃PO₄ (60 mg, 0.3 mmol), Pd(PPh₃)₄ (15 mg, 0.013 mmol), DME and H₂O. The reaction time was 30 min. For the SPE purification, a mixture of MeOH/H₂O 70:30 v/v (100 mL) was used. The HPLC separation was completed with MeOH/H₂O 60:40 v/v. Deprotection by Method B. The final product obtained as off-white crystalline powder (37 mg, 0.12 mmol, 44%).

M.p.: 207.1-209.7° C.

HPLC: tᵣ=18.43 min

¹H NMR (500 MHz, CDCl₃) δ [ppm] 8.86 (dd, ⁴J=1.9 Hz, ³J=4.1 Hz, 1H, H7′); 8.15 (dd, ⁴J=1.9 Hz, ³J=8.2 Hz, 1H, H5′); 7.85 (dd, ⁴J=1.3 Hz, ³J=7.3 Hz, 1H, H2′); 7.78 (dd, ⁴J=1.3 Hz, ³J=7.3 Hz, 1H, H4′); 7.58 (d, ³J=73 Hz, 1H, H4); 7.56 (t, ³J=7.3 Hz, 1H, H3′); 7.35 (dd, ³J=4.1 Hz, ³J=8.2 Hz, 1H, H6′); 6.16 (d, ³J=7.3 Hz, 1H, H5); 4.21 (d, ²J=15.7 Hz, 1H, H10β); 3.96 (dd, ³J=6.3 Hz, ²J=15.7 Hz, 1H, H10α); 3.13 (d, ²J=12.3 Hz, 1H, H13_{A}); 3.08 (s br, 2H, H11_{B}); 3.02 (d, ²J=12.3 Hz, 1H, H13_{B}); 2.95 (s br, 1H, H7); 2.33 (s br, 1H, H9), 1.96 (t, ²J=13.2 Hz, 2H, H8), 1.83 (br s, NH)

¹³C NMR (125 MHz, CDCl₃) δ [ppm] 162.5 (C═O, C2); 150.4 (C6); 149.9 (C7′); 146.5 (C8′a); 139.8 (C4); 139.6 (C1′); 136.5 (C3); 136.4 (C5′); 131.0 (C2′); 128.6 (C4′a); 127.7 (C4′); 126.2 (C3′); 120.8 (C6′); 104.8 (C5); 53.9 (C13); 53.0 (C11); 50.1 (C10); 35.8 (C7); 27.9 (C9); 26.4 (C8)

MS (EI) m/z 317.2 (100), 273.1 (50), 261.1 (40), 245.1 (20), 231.1 (10), 167.1 (15)

| | | |
|---|---|---|
| HRMS for C₂₀H₁₉N₃O | calc. | 317.1528 |
| | found | 317.1532 |

IUPAC 9-quinolin-8-yl-1,2,3,4,5,6-hexahydro-1,5-methano-pyridino[1,2-a]diazocin-8-one

### 3-(1-Methyl-1H-pyrazol-4′-yl)-cytisine 122e

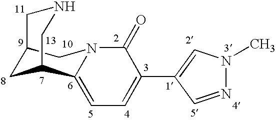

The Suzuki reaction was performed according to the general method with 3-bromo-N-tBOC-cytisine 81 (100 mg, 0.27 mmol), 1-methyl-4-(4,4,5,5-tetramethyl-1,3,2-dioxaborolan-2-yl)-1H-pyrazole (84 mg, 0.41 mmol), K₃PO₄ (126 mg, 0.6 mmol), Pd(PPh₃)₄ (30 mg, 0.027 mmol), DME and H₂O. The reaction time was 30 min. For the SPE purification, a mixture of MeOH/H₂O 60:40 v/v (100 mL) was used. The HPLC separation was completed with MeOH/H₂O 50:50 v/v. Deprotection by Method B. The final product obtained as yellow crystalline powder (51 mg, 0.19 mmol, 70%).

M.p.: 113.4° C.

HPLC: tᵣ=15.32 min

¹H NMR (500 MHz, CDCl₃) δ [ppm] 8.29 (s, 1H, H5′); 7.80 (s, 1H, H2′); 7.56 (d, ³J=7.3 Hz, 1H, H4); 6.05 (d, ³J=7.3 Hz, 1H, H5); 4.18 (d, ²J=15.7 Hz, 1H, H10β); 3.94 (dd, ³J=6.6 Hz, ²J=15.7 Hz, 1H, H10α); 3.90 (s, 3H, CH₃); 3.09 (d, ²J=12.0 Hz, 1H, H11_{A}); 3.03 (dd, ³J=2.2 Hz, ²J=12.0 Hz, 1H, H13_{A}); 2.99 (d, ²J=12.0 Hz, 1H, H13_{B}); 2.96 (d, ²J=12.0 Hz, 1H, H11_{B}); 2.90 (s br, 1H, H7), 2.33 (s br, 1H, H9), 1.94 (s br, 2H, H8)

¹³C NMR (125 MHz, CDCl₃) δ [ppm] 161.2 (C═O, C2); 148.0 (C6); 136.8 (C2′); 132.4 (C4); 129.9 (C5′); 119-8 (C3); 117.8 (C1′); 105.1 (C5); 53.9 (C13); 52.9 (C11); 50.1 (C10); 38.9 (CH₃); 35.5 (C7); 27.9 (C9); 26.4 (C8)

MS (EI) m/z 270.1 (100), 227.1 (60), 214.1 (10), 189.1 (20), 135.1 (5), 82.1 (5)

| | | |
|---|---|---|
| HRMS for C₁₅H₁₈N₄O | calc. | 270.1481 |
| | found | 270.1480 |

IUPAC 9-(1-Methyl-1H-pyrazol-4-yl)-1,2,3,4,5,6-hexahydro-1,5-methano-pyridino[1,2-a]diazocin-8-one

### (1H-Indol-5′-yl)-cytisine 123e

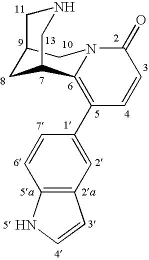

The Suzuki reaction was performed according to the general method with 5-bromo-N-tBOC-cytisine 82 (100 mg, 0.27 mmol), 5-indolylboronic acid (65 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification, a mixture of MeOH/H₂O 70:30 v/v (100 mL) was used. The HPLC separation was completed with MeOH/H₂O 60:40 v/v. Deprotection by Method B. The final product obtained as yellowish crystalline powder (21 mg, 0.07 mmol, 26%).

M.p.: 176.5-177.5° C.

HPLC: tᵣ=15.55 min

¹H NMR (500 MHz, CDCl₃) δ [ppm] 8.53 (s br, 1H, 5′-NH; 7.44 (t, ⁴J=0.9 Hz, 1H, H2′); 7.39 (dt, ⁴J=0.9 Hz, ³J=7.0 Hz, 1H, H7′); 728 (d, ³J=9.1 Hz, 1H, H4); 726 (t, ³J=2.8 Hz, 1H, H4′); 6.98 (dd, ³J=1.6 Hz, ³J=7.0 Hz, 1H, H6′); 6.53 (tt, ⁴J=0.9 Hz, ³J=2.8 Hz, 1H, H3′); 6.49 (d, ³J=9.1 Hz, 1H, 3); 4.23 (d, ²J=15.7 Hz, 1H, H10β); 3.97 (dd, ³J=6.9 Hz, ²J=15.7 Hz, 1H, H10α); 3.14 (s br, 1H, H7); 3.08 (d, ²J=12.3 Hz, 1H, H11_{A}); 2.93 (d, ²J=123 Hz, 1H, H11_{B}); 2.87 (d, ²J=12.3 Hz, 1H, H13_{A}); 2.64 (dd, ³J=2.5 Hz, ²J=12.3 Hz, 1H, H13_{B}); 2.30 (s br, 1H, H9); 1.93 (d br, ²J=12.7 Hz, 1H, H8_{A}); 1.81 (d br, ²J=12.7 Hz, 1H, H8_{B})

¹³C NMR (125 MHz, CDCl₃) δ [ppm] 163.2 (C═O, C2); 147.6 (C6); 142.2 (C4); 135.0 (C5′a); 129.8 (C1′); 128.0 (C2′a); 125.1 (C4′); 123.9 (C7′); 121.7 (C2′); 120.5 (C5); 115.7 (C3); 111.1 (C6′); 102.6 (C3′); 52.9 (C13); 52.2 (C11); 50.4 (C10); 31.6 (C7); 27.4 (C9); 26.4 (C8)

MS (EI) m/z 305.1 (100), 262.1 (70), 249.1 (25), 235.0 (20), 221.0 (15), 206.1 (10), 154.1 (10)

| | | |
|---|---|---|
| HRMS for C₁₉H₁₉N₃O | calc. | 305.1528 |
| | found | 305.1533 |

IUPAC 11-(1H-indol-5-yl)-1,2,3,4,5,6-hexahydro-1,5-methano-pyridino[1,2-a]diazocin-8-one

### 5-(3′,4′-Methylenedioxy-phenyl)-cytisine 124e

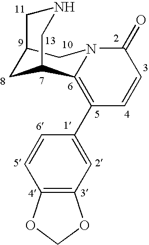

The Suzuki reaction performed according to the general method with 5-bromo-N-tBOC-cytisine 82 (100 mg, 0.27 mmol), 3,4-methylenedioxyphenylboronic acid (68 mg, 0.41 mmol), Ba(OH)₂*8H₂O (185 mg, 0.6 mmol), Pd(PPh₃)₄ (30 mg, 0.027 mmol), DME and H₂O. The reaction time was 60 min. For the SPE purification, a mixture of MeOH/H₂O 70:30 v/v (100 mL) was used. The HPLC separation was completed with MeOH/H₂O 60:40 v/v. Deprotection by Method B. The final product obtained as off-white crystalline powder (21 mg, 0.07 mmol, 25%).

M.p.: 94.5-96.5° C.

HPLC: tᵣ=18.68 min

¹H NMR (500 MHz, CDCl₃) δ [ppm] 7.17 (d, ³J=9.3 Hz, 1H, H4); 6.80 (d, ³J=7.9 Hz, 1H, H5′); 6.65 (d, ⁴J=1.8 Hz, 1H, H2′); 6.63 (dd, ⁴J=1.8 Hz, ³J=7.9 Hz, 1H, H6′); 6.45 (d, ³J=9.3 Hz, 1H, H3); 5.97 (s, 2H, CH₂); 4.18 (d, ²J=15.7 Hz, 1H, H10β); 3.93 (dd, ³J=6.6 Hz, ²J=15.7 Hz, 1H, H10α); 3.08 (d, ²J=12.0 Hz, 1H, H11_{A}; s br, ovl., 1H, H7); 2.92 (d, ²J=12.0 Hz, 1H H11_{B}); 2.82 (d, ²J=12.0 Hz, 1H, H13_{A}); 2.72 (dd, ³J=2.2 Hz, ²J=12.0 Hz, 1H, H13_{B}); 2.30 (s br, 1H, H9); 1.95 (d br, ²J=12.9 Hz, 1H, H8_{A}); 1.84 (d br, ²J=12.9 Hz, 1H, H8_{B})

¹³C NMR (125 MHz, CDCl₃) δ [ppm] 163.1 (C═O, C2); 147.8 (C6); 147.7 (C3′); 146.9 (C4′); 141.5 (C4); 132.0 (C1′); 123.1 (C6′); 118.8 (C5); 116.0 (C3); 110.2 (C2′); 108.4 (C5′); 101.2 (CH₂), 52.9 (C13); 52.1 (C11); 50.4 (C10); 31.6 (C7); 27.4 (C9); 26.4 (C8)

MS (EI) m/z 310.1 (100), 267.0 (70), 254.0 (20), 229.0 (25), 155.0 (10), 82.0 (10)

| | | |
|---|---|---|
| HRMS for C₁₈H₁₈N₂O₃ | calc. | 310.1317 |
| | found | 310.1318 |

IUPAC 11-(benzo[1,3]dioxol-5-yl)-1,2,3,4,5,6-hexahydro-1,5-methano-pyridino[1,2-a]diazocin-8-one

### 5-(Pyridin-3′-yl)-cytisine 125e

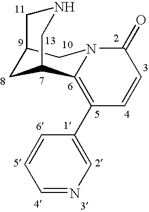

The Suzuki reaction was performed according to the general method with 5-bromo-N-tBOC-cytisine 82 (100 mg, 0-27 mmol), 3-pyridineboronic acid (49 mg, 0.41 mmol), Ba(OH)₂*8H₂O (185 mg, 0.6 mmol), Pd(PPh₃)₄ (30 mg, 0.027 mmol), DMP and H₂O. The reaction time was 90 min. For the SPE purification, a mixture of MeOH/H₂O 60:40 v/v (100 mL) was used. The HPLC separation was competed with MeOH/H₂O 50:50 v/v. Deprotection by Method B. The final product obtained as yellow crystalline powder (23 mg, 0.09 mmol, 32%).

M.p.: 70.4-72.0° C.

HPLC: tᵣ=14.52 min

¹H NMR (500 MHz, CDCl₃) δ [ppm] 8.58 (dd, ⁴J=1.6 Hz, ³J=5.0 Hz, 1H, H4′); 8.49 (d, ⁴J=1.9 Hz, 1H, H2′); 7.55 (dt, ⁴J1.9 Hz, ³J7.9 Hz, 1H, H6′); 733 (ddd, ⁵J=0.6 Hz, ³J=5.0 Hz, ³J=7.9 Hz, 1H, H5′); 7.18 (d, ³J=9.2 Hz, 1H, H4); 6.52 (d, ³J=9.2 Hz, 1H, H3); 4.21 (d, ²J=15.6 Hz, 1H, H10β); 3.95 (dd, ³J=6.6 Hz, ²J=15.6 Hz, 1H, H10β); 3.14 (d, ²J=12.0 Hz, 1H, H11_{A}); 2.97 (s br, 1H, H7); 2.93 (d, ²J=12.0 Hz, 1H, H11_{B}), 2.79 (d, ²J=12.0 Hz, 1H, H13_{A}); 2.71 (dd, ³J=2.5 Hz, ²J=12.0 Hz, 1H, H13_{B}); 2.35 (s br, 1H, H9), 1.94 (d, ²J=13.0 Hz, 1H, H8_{A}), 1.86 (d, ²J=13.0 Hz, 1H, H8_{B})

¹³C NMR (125 MHz, CDCl₃) δ [ppm] 163.0 (C═O, C2); 150.5 (C6); 148.8 (C2′); 148.1 (C6); 141.0 (C4); 137.2 (C6′); 134.3 (C1′); 123.5 (C5′); 116.7 (C3); 115.3 (C5), 52.7 (C13); 51.8 (C11); 50.4 (C10); 31.6 (C7); 27.2 (C9); 26.2 (C8)

MS (EI) m/z 267.1 (100), 224.1 (95), 211.1 (20), 186.1 (15), 156.1 (10), 82.0 (5)

| | | |
|---|---|---|
| HRMS for C₁₆H₁₇N₃O | calc. | 267.1371 |
| | found | 267.1377 |

IUPAC 11-pyridin-3-yl-1,2,3,4,5,6-hexahydro-1,5-methano-pyridino[1,2-a]diazocin-8-one 5-(1-Methyl-1H-pyrazol-4′-yl)-cytisine 126e

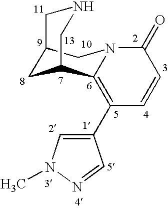

The Suzuki reaction was performed according to the general method with 5-bromo-N-tBOC-cytisine 82 (100 mg, 0.27 mmol), 1-methyl-4-(4,4,5,5-tetramethyl-1,3,2-dioxaborolan-2-yl)-1H-pyrazole (84 mg, 0.41 mmol), Ba(OH)₂*8H₂O (185 mg, 0.6 mmol), Pd(PPh₃)₄ (30 mg, 0.027 mmol), DME and H₂O. The reaction time was 60 min. For the SPE purification, a mixture of MeOH/H₂O 60:40 (100 mL) was used. The HPLC separation was completed with MeOH/H₂O 50:50 v/v. Deprotection by Method B. The final product obtained as yellow crystalline powder (14 mg, 0.05 mmol, 19%).

M.p.: n.d.

HPLC: tᵣ=12.65 min

¹H NMR (500 MHz, CDCl₃) δ [ppm] 7.38 (s, 1H, H5′); 7.26 (s, 1H, H2′); 7.22 (d, ³J=9.1 Hz, 1H, H4); 6.46 (d, ³J=9.1 Hz, 1H, H3); 4.17 (d, ²J=15.7 Hz, 1H, H10β); 3.95 (dd, ³J=6.9 Hz, ²J=15.7 Hz, 1H, H10α); 3.91 (s, 3H, CH₃); 3.13 (s br, 1H, H7); 3.08 (d, ²J=12.3 Hz, 1H, H11_{A}); 2.98 (d, ²J=12.3 Hz, 1H, H11_{B}); 2.93 (d, ²J=12.3 Hz, 1H, H13_{A}); 2.87 (dd, ³J=2.5 Hz, ²J=12.3 Hz, 1H, H11_{B}); 2.31 (s, 1H, H9); 1.88 (s, 2H, H8)

¹³C NMR (125 MHz, CDCl₃) δ [ppm] 163.1 (C═O, C2); 148.2 (C6); 142.0 (C4); 139.1 (C2′); 129.1 (C5′); 118.5 (C1′); 116.3 (C3); 109.4 (C5); 52.9 (C13); 52.4 (C11); 50.4 (C10); 39.1 (CH₃); 31.8 (C7); 27.3 (C9); 26.3 (C8)

MS (EI) m/z 270.1 (100), 227.1 (68), 214.1 (25), 200.1 (15), 189.1 (30), 146.0 (20), 119.1 (10), 82.0 (10)

| | | |
|---|---|---|
| HRMS for C₁₅H₁₈N₄O | calc. | 270.1481 |
| | found | 270.1482 |

IUPAC 11-(1-methyl-1H-pyrazol-4-yl)-1,2,3,4,5,6-hexahydro-1,5-methano-pyridino[1,2-a]diazocin-8-one

### 5-Bromo-3-phenyl-cytisine 128e

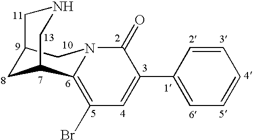

The Suzuki reaction was performed according to the general method with 3,5-dibromo-N-tBOC-cytisine 83 (121 mg, 0.27 mmol), 3-phenylboronic acid (50 mg, 0.41 mmol), Na₂CO₃ (64 mg, 0.6 mmol), Pd(PPh₃)₄ (30 mg, 0.027 mmol), DME and H₂O. The reaction time was 30 min. For the SPE purification, a mixture of MeOH/H₂O 80:20 v/v (100 mL) was used. The HPLC separation was completed with MeOH/H₂O 70:30 v/v. Deprotection by Method A. The final product obtained as off-white crystalline powder (38 mg, 0.11 mmol, 33%).

M.p.: 126.1-127.0° C.

HPLC: tᵣ=25.23 min

¹H NMR (500 MHz, CDCl₃) δ [ppm] 7.65 (d, ³J=7.2 Hz, 2H, H2′+H4′); 7.60 (s, 1H, H4); 7.37 (t, ³J=7.2 Hz, 2H, H3′+H5′); 7.30 (tt, ⁴J=1.3 Hz, ³J=7.2 Hz, 1H, H4′); 4.13 (d, ²J=15.5 Hz, 1H, H10β); 3.96 (dd, ³J=6.6 Hz, ²J=15.5 Hz, 1H, H10α); 3.36 (s br, 1H, H7); 3.18 (d, ²J=12.3 Hz, 1H, H13_{A}); 3.08 (d, ²J=12.3 Hz, 1H, H11_{A}); 2.99 (d, ²J=12.3 Hz, 1H, H11_{B}); 2.96 (d, ²J=12.3 Hz, 1H, H13_{B}); 2.34 (s br, 1H, H9); 1.98 (d br, ²J=12.9 Hz, 1H, H8_{A}); 1.94 (d br, ²J=12.9 Hz, 1H, H8_{B})

¹³C NMR (125 MHz, CDCl₃) δ [ppm] 161.2 (C═O, C2); 147.0 (C6); 140.4 (C4); 136.0 (C1′); 128.6 (C3); 128.6 (C2′+C6′); 128.1 (C3′+C5′); 127.8 (C4′); 98.8 (C5); 52.7 (C13); 51.3 (C11); 50.3 (C10); 34.9 (C7); 27.6 (C9); 26.4 (C8)

MS (EI) m/z 344.1 (100), 302.0 (80), 277.1 (20), 263.0 (40), 162.1 (40), 82.1 (20), 57.1 (10)

| | | |
|---|---|---|
| HRMS for C₁₇H₁₇BrN₂O | calc. | 344.0524 |
| | found | 344.0529 |

IUPAC 11-bromo-9-phenyl-1,2,3,4,5,6-hexahydro-1,5-methano-pyridino[1,2-a]diazocin-8-one

### 5-Bromo-3-(pyridin-3′-yl)-cytisine 129e

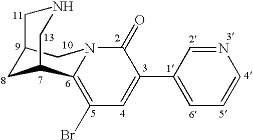

The Suzuki reaction was performed according to the general method with 3,5-dibromo-N-tBOC-cytisine 83 (121 mg, 0.27 mmol), 3-pyridineboronic acid (49 mg, 0.41 mmol), K₃PO₄ (60 mg, 0.6 mmol), Pd(PPh₃)₄ (30 mg, 0.027 mmol), DME and H₂O. The reaction time was 60 min. For the SPE purification, a mixture of MeOH/H₂O 70:30 v/v (100 mL) was used. The HPLC separation was completed with MeOH/H₂O 70:30 v/v. Deprotection by Method B. The final product obtained as yellow crystalline powder (39 mg, 0.11 mmol, 41%).

M.p.: 87.6-92.3° C.

HPLC: tᵣ=26.98 min

¹H NMR (500 MHz, CDCl₃) δ [ppm] 8.81 (d, ⁴J=1.6 Hz, 1H, H2′); 8.55 (dd, ⁴J=2.0 Hz, ³J=5.1 Hz, 1H, H4′); 8.13 (dt, ⁴J=2.0 Hz, ³J=8.0 Hz, 1H, H6′); 7.67 (s, 1H, H4); 7.33 (dd, ³J=5.1 Hz, ³J=8.0 Hz, 1H, H5′); 4.16 (d, ²J=15.8 Hz, 1H, H10β); 4.00 (dd, ³J=6.6 Hz, ²J=15.8 Hz, 1H, H10α); 3.42 (s br, 1H, H7); 3.25 (d, ²J=12.0 Hz, 1H, H13_{A}); 3.12 (d, ²J=12.0 Hz, 1H, H11_{A}); 3.03 (d, ²J=12.0 Hz, 1H11_{B}); 2.98 (dd, ³J=2.2 Hz, ²J=12.0 Hz, 1H, H13_{B}); 2.39 (s br, 1H, H9); 2.02 (d br, ²J=13.2 Hz, 1H, H8_{A}); 1.96 (d br, ²J=13.2 Hz, 1H, H8_{B})

¹³C NMR (125 MHz, CDCl₃) δ [ppm] 161.0 (C═O, C2); 149.1 (C6); 148.8 (C2′); 148.3 (C4′); 140.6 (C4); 136.1 (C6′); 131.9 (C1′); 125.3 (C3); 122.9 (C5′); 98.7 (C5); 52.7 (C13); 51.4 (C11); 50.4 (C10); 35.0 (C7); 27.5 (C9); 26.4 (C8)

MS (EI) m/z 347.0/345.0 (90), 303.0 (100), 289.0 (20), 264.0 (40), 223.1 (10), 194.1 (10), 168.1 (5), 82.0 (10)

| | | |
|---|---|---|
| HRMS for C₁₆H₁₆BrN₃O | calc. | 345.0477 |
| | found | 345.0475 |

IUPAC 11-bromo-9-pyridin-3-yl-1,2,3,4,5,6-hexahydro-1,5-methano-pyridino[1,2-a]diazocin-8-one

Synthesis of Novel nAChR Ligands Based on Choline

General Procedure for the Synthesis of Phenylcarbamates

Equimolar amounts of the amino alcohol and appropriate phenylisocyanate were stirred in toluene (10 mL) under argon atmosphere at 50° C. for 1.5-4 hours. The solvent was evaporated and the resulting oily residue was purified by flash chromatography on a small amount of silica gel (max. 50 mg) eluting with CH₂Cl₂/MeOH (95:5).

### (3-Methyl-phenyl)-carbamic (S)—(−)-1-methyl-pyrrolidin-2-yl-methyl ester 136

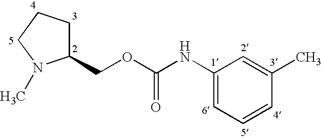

The synthesis was performed according to the general method with (S)-(−)-1-methyl-2-pyrrolidinylmethanol (0.24 mL, 2 mmol) and m-tolylisocyanate (0.26 mL, 2 mmol). The filial product was obtained as a colourless oil (206 mg, 0.83 mmol, 42%).

M.p.: 62.1-62.3° C.

[α]_{D}²⁰+13295° (c 0.02, MeOH)

IR (KBr): 1565, 1710, 2799, 2855, 3053 cm⁻¹

¹H NMR (500 MHz CDCl₃) δ 7.21 (s, 1H, H2′); 7.11-7.17 (m, 2H, H5′+H6′); 6.84 (d, ³J=6.9 Hz, 1H, H4′); 6.67 (s, 1H, NH); 4.22 (dd, ³J=4.4 Hz, ²J=11.0 Hz, 1H, CH_{2A}); 4.05 (dd, ³J=4.7 Hz, ²J=11.4 Hz, 1H, CH_{2B}); 3.08 (dt, ³J=1.9 Hz, ³J=8.2 Hz, 1H, H2); 2.43-2.48 (m, 1H, H_{5A}); 2.40 (s, 3H, CH₃); 2.30 (s, 3H, N—CH₃); 2.20-2.26 (dt, ³J=7.6 Hz, ³J=9.5 Hz, 1H, H5_{B}); 1.88-1.93 (m, 1H, H4_{A}); 1.64-1.81 (m, 3H, H3+H4_{B})

¹³C NMR (125 MHz, CDCl₃) δ 153.5 (C═O); 139.0 (C1′); 137.8 (C3′); 128.8 (C5′); 124.2 (C4′); 119.2 (C2′); 115.6 (C6′); 66.0 (CH₂); 64.2 (C2); 57.5 (C5); 41.2 (N—CH₃); 27.9 (C3); 22.7 (C4); 21.5 (CH₃)

MS (EI) m/z 248.1 (20) [M], 97 (20), 84 (100)

| | |
|---|---|
| Anal. calcd. for C₁₄H₂₀N₂O₂ (248.33) | C, 67.71; H, 8.05; N, 11.28 |
| Found | C, 67.21; H, 8.12; N, 10.65 |

(3-Bromophenyl)-carbamic (S-(−)-1-methyl-pyrrolidin-2-yl-methyl ester 137

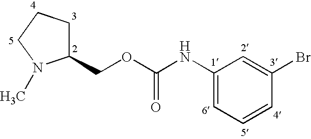

The synthesis was performed according to the general method with (S)-(−)-1-methyl-2-pyrrolidinylmethanol (0.24 mL, 2 mmol) and m-bromophenylisocyanate (0.25 mL, 2 mmol). The final product was obtained as a colourless oil, which crystallised on standing (610 mg, 1.94 mmol, 97%).

M.p.: 49.0-49.6° C.

IR (KBr): 2942, 2857, 1714, 1596 cm⁻¹

[α]_{D}²+1.3295° (c 0.02, MeOH)

¹H NMR (500 MHz, CDCl₃) 7.62 (s, 1H, H2′); 7.23 (s br, 1H, H6′); 7.10-7.16 (m, 2H, H4′+H5′); 6.78 (br s, 1H, NH); 422 (dd, J=4.4 Hz, J=11.0 Hz, 1H, CH₂ₐ); 4.10 (dd, J=4.4 Hz, J=11.0 Hz, 1H, CH_{2B}); 3.07 (dt, J=1.9 Hz, J=7.9 Hz, 1H, H2); 2.42-2.47 (m, 1H, H5_{A}); 2.39 (s, 3H, N—CH₃); 2.20-2.25 (dt, J=7.3 Hz, J=9.5 Hz, 1H, H5_{B}); 1.82-1.93 (m, 1H, H4_{A}); 1.63-1.82 (m, 3H, H3+H4_{B})

¹³C NMR (125 MHz, CDCl₃) δ 153.2 (C═O); 139.2 (C1′); 130.3 (C5′); 126.3 (C4′); 122.7 (C3′); 121.4 (C2′); 117.0/C6′); 66.3 (CH₂); 64.1 (C2); 57.5 (C5); 41.1 (CH₃); 27.8 (C3); 22.7 (C4)

MS (EI) m/z 312.1 (20) [M−H⁺], 198.9 (10), 97 (20), 84.0 (100)

| | |
|---|---|
| Anal. calc. for C₁₃H₁₇BrN₂O₂ (313.202) | C, 49.85; H, 5.47; N, 8.94 |
| Found | C, 49.01; H, 5.57; N, 8.54 |

### (3-Methyl-phenyl)-carbamic 1-methyl-piperidin-2-yl-methyl ester 138

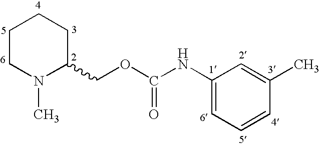

The synthesis was performed according to the general method with 2-hydroxymethyl-N-methylpiperidine (0.26 mL, 2 mmol) and m-tolylisocyanate (0.26 mL, 2 mmol). The final product was obtained as a yellowish oil, which crystallised on standing (210 mg, 0.6 mmol, 30%).

M.p.: 73.5-75.5° C.

IR (KBr): 2937, 2852, 2803, 1732 cm⁻¹

¹H NMR (500 MHz, CDCl₃) δ 7.23 (s, 1H, H2′); 7.15-7.19 (m, 2H, H5′+H6′); 6.87 (d, ³J=6.9 Hz, 1H, H4′); 6.81 (s, 1H, NH); 4.25 (dd, ³J=4.1 Hz, ²J=11.7 Hz, 1H, CH_{2A}); 4.23 (dd, ³J=3.2 Hz, ²J=11.7 Hz, 1H, CH_{2B}); 2.88-2.93 (m, 1H, H2); 2.34 (s, 3H, N—CH₃); 2.32 (s, 3H, CH₃); 2.08-2.13 (m, 2H, H6); 1.75-1.82 (m, 1H, H3_{A}); 1.50-1.69 (m, 4H, H4+H5); 1.24-1.34 (m, 1H, H3_{B})

¹³C NMR (125 MHz, CDCl₃) δ 153.6 (C═O); 139.0 (C1′); 137.8 (C3′); 128.9 (C5′); 124.2 (C4′); 119.1 (C2′); 115.6 (C6′); 66.2 (CH₂); 62.9 (C2); 57.4 (C6); 43.2 (N—CH₃); 29.2 (C3); 25.8 (C5); 24.2 (C4); 21.5 (CH₃)

MS (EI) m/z 262.2 (38) [M], 134 (10), 98.1 (100), 77 (10)

| | |
|---|---|
| Anal. calcd. for C₁₅H₂₂N₂O₂ (262.353) | C, 68.67; H, 8.45; N, 10.67 |
| Found | C, 68.78; H: 8.47; N, 10.13 |

### (3-Bromo-phenyl)-carbamic 1-methyl-piperidin-2-yl-methyl ester 139

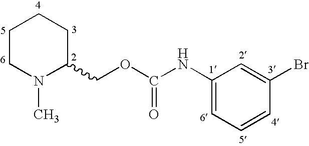

The synthesis was performed according to the general method with 2-hydroxymethyl-N-methylpiperidine (0.26 mL, 2 mmol) and m-bromophenylisocyanate (0.25 mL, 2 mmol). The final product was obtained as yellowish oil, which crystallised on standing (555.2 mg, 1.7 mmol, 85%).

M.p.: 67.3-68.1° C.

IR (KBr): 2936, 2856, 2794, 1729, 1705, 1533 cm⁻¹

¹H NMR (500 MHz, CDCl₃), 7.66 (s, 1H, H2′); 7.26 (d, ³J=7.8 Hz, 1H, H6′); 7.12-7.20 (m, 2H, H4′+H5′); 7.04 (s, 1H, NH), 4.28 (dd, ³J=3.8 Hz, ²J=11.7 Hz, 1H, CH_{2A}); 4.20 (dd, ³J=3.2 Hz, J=11.7 Hz, 1H, CH_{2B}); 2.91 (d, ³J=11.7 Hz, 1H, H2); 2.35 (s, 3H, N—CH₃); 2.06-2.12 (m, 2H, H6); 1.76-1.80 (m, 1H, H_{3A}); 1.51-1.68 (m, 4H, H4+H5); 1.25-1.35 (m, 1H, H3_{B})

¹³C NMR (125 MHz, CDCl₃) δ 153.2 (C≡O); 139.3 (C1′); 130.2 (C5′); 126.3 (C4′); 122.7 (C3′); 121.3 (C2′); 116.9 (C6′); 66.3 (CH₂); 62.8 (C2); 57.3 (C6); 43.0 (N—CH₃); 29.1 (C6); 25.6 (C5); 24.1 (C4)

MS (EI) m/z 326.1 (10) [M⁺], 196.9 (10), 98 (100)

| | |
|---|---|
| Anal. calcd. for C₁₄H₁₉Br N₂O₂ (327.218) | C, 51.39; H, 5.85; N, 8.56 |
| Found | C, 50.81; H, 6.00; N, 8.12 |

### m-Tolyl-carbamic acid 1-aza-bicyclo[2.2.2]oct-3-yl ester 140

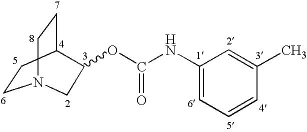

The synthesis was performed according to the general method with 3-quinuclidinole (254 mg, 2 mmol) and m-tolylisocyanate (0.26 mL, 2 mmol). The resulting oily residue was purified by column chromatography eluting with CH₂Cl₂/MeOH (90:10→90:50 v/v) and crystallised from diethyl ether. The final product was obtained as white crystalline powder (150 mg, 0.57 mmol, 28%).

M.p.: 150.1-150.4° C.

IR (KBr); 2938, 2866, 2780, 1710, 1598, 1559 cm⁻¹

¹H NMR (500 MHz, CDCl₃) δ 7.29 (s, 1H, H2′); 7.26 (d, ³J=8.2 Hz, 1H, H6′); 7.18 (t, ³J=7.6 Hz, 1H, H5′); 6.88 (d, ³J=7.6 Hz, 1H, H4′); 4.84-4.85 (m, 1H, H3_{A}); 3.34 (qui, J=1.7 Hz, 1H, H3_{B}); 3.25 (ddd, J=2.4 Hz, J=8.4 Hz, J=14.7 Hz, 1H, H2); 2.77-2.95 (m, 5H, H2+H6+H8); 2.34 (s, 3H, CH₃); 2.10-2.14 (m, 1H, H4); 1.98-2.06 (m, 1H, H5 or H7); 1.78-1.85 (m, 1H, H5 or H7); 1.66-1.72 (m, 1H, H5 or H7); 1.52-1.58 (m, 1H, H5 or H7)

¹³C NMR (125 MHz, CDCl₃): δ 156.0 (C≡O); 140.3 (C1′); 140.0 (C3′); 130.0 (C5′); 125.1 (C4′); 120.8 (C2′); 117.3 (C6′); 72.5 (C3); 56.4 (C2); 48.3 (C8); 47.3 (C6); 26.8 (C4); 25.1 (C7); 21.9 (C5); 20.4 (CH₃)

MS (EI) m/z 260.2 (25) [M], 147.1 (10), 134.0 (32), 122.0 (40), 105.0 (100), 82 (18), 77 (25)

| | |
|---|---|
| Anal. calcd. for C₁₅H₂₀N₂O₂ (260.34) | C, 68.20; H, 7.74; N, 10.76 |
| Found | C, 68.37; H, 7.72; N, 10.98 |

### 3-Bromo-carbamic acid 1-aza-bicyclo[2.2.2]oct-3-yl ester 141

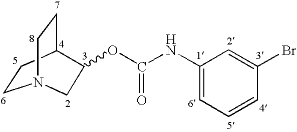

The synthesis was performed according to the general method with 3-quinuclidinole (254 mg, 2 mmol) and 3-bromophenylisocyanate (0.25 mL, 2 mmol). The oily residue was crystallised from diethyl ether to yield the final product as white crystals (488 mg, 1.5 mmol, 75%).

M.p.: 162.2-162.4° C.

IR (KBr): 3163, 2943, 2866, 1722, 1595 cm⁻¹

¹ NMR (500 MHz, CDCl₃+TMS) δ 7.65 (s, 1H, H2′); 7.26 (s, 1H, H6′); 7.13-7.20 (m, 2H, H4′+H5′); 7.08 (s, 1H, NH); 4.80-4.91 (m, 1H, H3); 3.27 (ddd, J=1.9 Hz, J=8.4 Hz, J=14.5 Hz, 1H, H2_{A}); 2.73-2.96 (m, 5H, H2_{B}+H6+H8); 2.09-2.18 (s, 1H, H4); 1.81-1.87 (m, 1H, H5 or H7); 1.68-1.75 (m, 1H, H5 or H7); 1.55-1.61 (m, 1H, H5 or H7); 1.40-1.46 (m, 1H, H5 or H7)

¹³C NMR (125 MHz, CDCl₃+TMS) δ 153.1 (C═O); 139.4 (C1′); 130.3 (C5′); 126.3 (C4′); 122.8 (C3′); 121.6 (C2′); 117.0 (C6′); 72.5 (C3); 55.4 (C2); 47.3 (C8); 46.5 (C6); 25.4 (C4); 24.5 (C7); 19.5 (C5)

MS (EI) m/z 324.1 (40) [M−H⁺], 126.1 (100), 109.0 (28), 82.0 (22)

| | |
|---|---|
| Anal. calc. for C₁₄H₁₇BrN₂O₂ (325.21) | C, 51.70; H, 5.27; N, 8.61 |
| Found | C, 51.05; H, 5.22; N: 8.60 |

### Biphenyl-3-yl-carbamic acid 1-aza-bicyclo[2.2.2]oct-3-yl ester 144

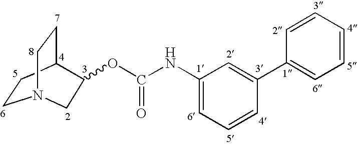

3-Bromo-carbamic acid 1-aza-bicyclo-[2.2.2]oct-3-yl ester 141 (325 mg, 1 mmol), phenylboronic acid (244 mg, 2 mmol), tetrakis-(triphenylphosphin)-palladium(0) (58 mg, 0.05 mmol), Na₂CO₃ (233 mg, 2.2 mmol), toluene (5 mL) and a magnetic stir bar were placed in a 10-mL microwave glass tube. The vessel was sealed with a septum and placed into the microwave cavity. Enhanced microwave irradiation of 100 W was used, the temperature being ramped from room temperature to 120° C. Once 120° C. was reached, the reaction mixture was held for 20 min. Then, the mixture was allowed to cool to room temperature, the reaction vessel was opened and the solvent was evaporated under pressure. The oily residue was purified by column chromatography on silica gel eluting with CH₂Cl₂/MeOH (80:20). The final product was crystallised from the mixture of diethyl ether/petroleum ether and obtained as a yellow crystalline powder (37.7 mg, 0.11 mmol, 22.7%).

M.p.: 201-202° C.

IR (KBr) 3189, 2934, 2868, 1716 cm⁻¹

¹H NMR (500 MHz, DMSO-d₆) δ 9.66 (s, 1H, NH); 7.79 (s, 1H, H2′); 7.58 (dt, ⁴J=1.4 Hz, ³J=7.1 Hz, 2H, H2″+H6″); 7.43-7.47 (m, 3H, H6′+H3″+H5″); 7.36 (tt, ⁴J=1.4 Hz, ³J=7.1 Hz, 1H, H4″); 7.35 (t, ³J=7.7 Hz, 1H, H5′); 7.25 (dt, ⁴J-1.5 Hz ³J=8.2 Hz, 1H, H4′); 4.68-4.71 (m, 1H, H3); 3.15 (ddd, J=1.8 Hz, J=7.9 Hz, J=14.5 Hz, 1H, H2_{A}); 2.57-2.72 (m, 5H, H2_{B}+H6+H8); 1.98 (sx, J=3.2 Hz, 1H, H4); 1.78-1.83 (m, 1H, H5 or H7); 1.59-1.65 (m, 1H, H5 or H7); 1.47-1.54 (m, 1H, H5 or H7), 1.33-1.39 (m, 1H, H5 or H7)

¹³C NMR (125 MHz, DMSO-d₆) δ153.6 (C═O); 140.9 (C3′); 140.4 (C1′); 139.9 (C1″); 129.4 C5′); 129.1 (C3″+C5′); 127.6 (C4″); 126.7 (C2″+C6″), 120.9 (C4′); 117.4 (C2′); 116.6 (C6′); 71.4 (C3); 55.3 (C2); 47.1 (C8); 46.1 (C6); 25.4 (C4); 24.4 (C7); 19.3 (C5)

MS (EI) m/z 322.2 (10) [M+]

Anal. calcd. for C₂₀H₂₂N₂O₂ (322.41): C, 74.50; H, 6.88; N, 8.69.
Found: C, 74.28; H, 6.48; N, 8.15.

### 3-Styryl)-phenyl]carbamic acid 1-aza-bicyclo-[2.2.2]oct-3-yl ester 145

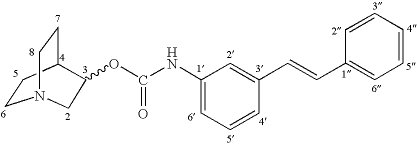

3-Bromo-carbamic acid 1-aza-bicyclo[2.2.2]oct-3-yl ester 141 (325 mg, 1 mmol), styrylboronic acid (300 mg, 2 mmol), tetrakis-triphenylphosphin)-palladium(0) (115.5 mg, 0.1 mmol), Na₂CO₃ (233 mg, 2.2 mmol), toluene (5 mL) and a magnetic stir bar were placed in a 10-mL microwave glass tube. The vessel was sealed with a septum and placed into the microwave cavity. Microwave irradiation of 60 W was used, the temperature being ramped from room temperature to 120° C. Once 120° C. was reached, the reaction mixture was held for 10 min. Then the mixture was allowed to cool to room temperature, the reaction vessel was opened and the solvent was evaporated under pressure. The oily residue was purified by column chromatography on silica gel eluting with CH₂Cl₂/MeOH (90:10). The final product was crystallised from the mixture of diethyl ether/petroleum ether and obtained as a yellow crystalline powder (101.1 mg, 0.2 mmol, 39%).

M.p.: 174-175° C.

IR (KBr): 3021, 2945, 2771, 2661, 2589, 1728, 1589, 1547, 1224, 960 cm⁻

¹H NMR (500 MHz, DMSO-d₆) δ 9.81 (s, 1H, NH); 7.70 (s, 1H, H2′); 7.59 (d, ³J=7.1 Hz, 2H, H2″+H6″); 7.44 (d, J=7.3 Hz, 1H, H6′); 7.37 (t, ³J=7.4 Hz, 3H, H3″+H5″+H5′); 7.26-7.29 (m, 2H, H4′+H4″); 7.20 (d, ³J=16.6 Hz, 1H, —CH═); 7.13 (d, ³J=16.6 Hz, 1H, —CH═); 4.93-5.00 (m, 1H, H3); 3.68 (ddd, J=2.1 Hz, J=8.4 Hz, J=13.7 Hz, 1H, H2_{A}); 3.15-3.25 (m, 5H, H2_{B}+H6+H8); 2.28 (sx, J=2.9 Hz, 1H, H4); 1.73-191 (m, 4H, H5+H7)

¹³C NMR (125 MHz, DMSO-d₆) δ 153.0 (C═O); 139.3 (C1′); 137.7 (C3′); 136.9 (C1″); 129.2, 128.8 (C3″+C5″); 128.7 (—CH═); 128.5 (C5′); 127.9 (C4″); 126.7 (C2″+C6″); 121.2 (C4′); 118.03 (C6′); 116.5 (C2′); 67.6 (C3); 53.1 (C2); 45.9 (C8); 45.1 (C6); 24.0 (C4); 20.2 (C7); 16.9 (C5)

MS (EI) m/z 348.2 (100) MI

| | |
|---|---|
| Anal. calcd. for C₂₂H₂₄N₂O₂ (348.45): | C, 75.83; H, 6.94; N, 8.04 |
| Found | C, 75.68; H, 6.96; N, 8.23 |

In Vitro Evaluation of Novel mAChRs Ligands

General Information

Membrane Preparation

Preparation of Rat Brains

Frozen rat brains were thawed slowly before the preparation of the P2 rat brain membrane fraction (30-60 min on ice, afterwards at room temperature). A single cut just behind the inferior colliculi was done to exclude the cerebellum and medulla. After the determination of the wet weight (1.32 g on average), the brains were pressed into a pulp using a syringe and homogenised in saccharose buffer with a glass teflon homogenizator (Potter, 10 seconds). The tissue was then centrifuged (1,000×g, 20 min, 4° C.), the supernatant (S1) aspirated with a Pasteur pipette and stored on ice. The P1 pellet was re-suspended in saccharose buffer and the centrifugation was repeated. The supernatant S1′ was collected and added to the supernatant S1. The combined supernatants were centrifuged (25,000×g, 20 min, 4° C.), the supernatant S2 was removed and the pellet P2 collected and diluted with HSS-buffer. The buffer volume added was calculated on the basis of the wet weight in a ratio 1:2.

The final pellet was stored in aliquots at −80° C. On the day of the experiment, the P2 membrane fraction was thawed, diluted with HSS-buffer (30-fold volume), homogenised and centrifuged (35,000×g, 10 min, 4° C.). The collected pellet was suspended in HSS-buffer and used in the radioligand binding experiments.

Preparation of Calf Adrenals

Frozen calf adrenals (−80° C.) were placed on ice for 30-60 min and allowed to thaw slowly before they were cut into small pieces. After determination of the wet weight (4-6 g), the tissue was homogenised in HSS-buffer (Ultraturrax at 750 rpm). The homogenate was centrifuged (30,000×g, 10 min, 4° C.), the pellets collected and washed. This procedure was repeated five times. The buffer volume used to re-suspend the pellets was calculated on the basis of the wet weight in a ratio 1:6.5.

The prepared tissues were stored in aliquots at −80° C. One hour before the experiments the tissues were slowly thawed, homogenised in HSS-buffer and centrifuged (25,000×g, 20 min, 4° C.). The pellets were re-suspended in fresh HSS-buffer and used for radioligand binding assays.

Preparation of Torpedo californica Electroplax

Frozen samples of Torpedo californica electric organ (−80° C.) were placed on ice for 30-60 min and allowed to thaw slowly before the membrane preparation. The tissue was homogenised in an ice-cold HSS-buffer (Ultraturrax at 750 rpm) and centrifuged (30,000×g, 10 min, 4° C.). The pellets were collected, washed four times with HSS-buffer through re-homogenization and centrifugation at the same settings. The remaining pellets were collected, re-suspended in HSS and stored in aliquots at −80° C.

One hour before the experiments the tissues were slowly thawed, homogenised in HSS-buffer and centrifuged (25,000×g, 20 min, 4° C.). The pellets were re-suspended in fresh HSS-buffer and used for radioligand binding assays.

Radioligand Binding Studies

Competition Assay Using (±)-[³H]Epibatidine ([³H]Epi) and Rat Brain P2-Fraction (α4β2*nAChR)

A dilution row of 6-9 concentrations of the test compound was prepared. Each assay sample, with a total volume of 500 μL contained 100 μL of the membrane protein (60 μg), 100 μL of (±)-[³H]epibatidine (0.5 nM), 100 μL of HSS-buffer and 200 μL of the test compound Non-specific binding was determined in the presence of 300 μM (−)-nicotine tartrate salt. The samples were homogenised and incubated for 90 min at 22° C. The incubation was terminated by vacuum filtration through glass fibre filters pre-soaked in 1% poly(ethyleneimine). The filter were rinsed three times with TRIS-buffer, punched out and transferred into 4 mL scintillation vials. The scintillation vials were filled with scintillation cocktail (2 mL) and the radioactivity was measured using a liquid scintillation counter.

Assays were carried out in duplicate, triplicates or quadruplicates.

Competition Assay Using [³H]Methyllycaconitine ([³H]MLA) and Rat Brain P2-Fraction (α7*nAChR)

A dilution row of 6-9 concentrations of the test compound was prepared. Each assay sample, with a total volume of 250 μL contained 50 μL of the test compound, 100 μL of [³H]MLA and 100 μL of the P2-membrane protein fraction (60-70 μg). Non-specific binding was determined in the presence of 1 μM MLA. The samples were homogenised and incubated for 120 min at 22° C. The incubation was terminated by vacuum filtration through glass fibre filters pre-soaked in 1% poly(ethyleneimine). The filters were rinsed three times with TRIS-buffer, punched out and transferred into 4 mL scintillation vials. The scintillation vials were filled with scintillation cocktail (2 mL) and the radioactivity was measured using a liquid scintillation counter.

Assays were carried out in duplicates, triplicates or quadruplicates.

Competition Assay Using (±)-[³H]Epibatidine ([³H]Epi) and Calf Adrenals Membrane Fraction (α3β4*nAChR)

A dilution row of 6-9 concentrations of the test compound was prepared. Each assay sample, with a total volume of 500 μL contained 200 μL of the test compound, 100 μL of (±)-[³H]epibatidine, 100 μL of the calf adrenal membrane protein fraction (60-70 μg) and 100 μM of HSS-buffer. Non-specific binding was determined in the presence of 300 μM (−)-nicotine tartrate salt. The samples were homogenised and incubated for 90 min at 22° C. The incubation was terminated by vacuum filtration through glass fibre filters presoaked in 1% poly(ethyleneimine). The filters were rinsed three times with TRIS-buffer, punched out and transferred into 4 mL scintillation vials. The scintillation vials were filled with scintillation cocktail (2 mL) and the radioactivity was measured using a liquid scintillation counter.

Assays were carried out in duplicates, triplicates or quadruplicates

Competition Assay Using (±)-[³H]Epibatidine ([³H]Epi) and Torpedo californica Electroplax ((α1)₂β1γδ nAChR)

A dilution row of 6-9 concentrations of the test compound was prepared. Each assay sample, with a total volume of 500 μL contained 200 μL of the test compound, 100 μL of (±)-[³H]epibatidine and 100 μL of the Torpedo californica electroplax fraction (60-70 μg). Non-specific binding was determined in the presence of 300 μM (−)-nicotine tartrate salt. The samples were homogenised and incubated for 90 min at 22° C. The incubation was terminated by vacuum filtration through glass fibre filters pre-soaked in 1% poly(ethyleneimine). The filters were rinsed three times with TRIS-buffer, punched out and transferred into 4 mL scintillation vials. The scintillation vials were filled with scintillation cocktail (2 mL) and the radioactivity was measured using a liquid scintillation counter.

Assays were carried out in duplicates, triplicate or quadruplicates. The results appear below.

TABLE 4
Binding affinity values (Kᵢ) for 3-aryl derivatives of cytisine 93e-100e
at α4β2*, α7*, α3β4* and (α1)₂β1γδ nACh receptor subtypes

| | | | | α3β4* | (α1)₂β1γδ |
|---|---|---|---|---|---|
| | | α4β2* | α7* | [³H]epi | [³H]epi |
| | | [³H]epi | [³H]MLA | calf | Torp. calif. |
| | | rat brain | rat brain | adrenals | electroplax |
| Structure | No. | Ki [nM]^{a} | Ki [nM]^{b} | Ki [nM]^{a} | Ki [nM]^{a} |
| 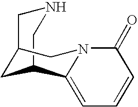 | 27 | 0.122 | 250 | 19 | 1,300 |
| 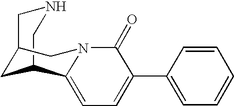 | 93e | 128 | >10,000 | >10,000 | >10,000 |
| 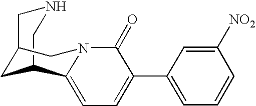 | 94e | 23 | >10,000 | >2,000 | >10,000 |
| 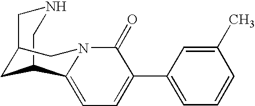 | 95e | 28 | >10,000 | >10,000 | >10,000 |
| 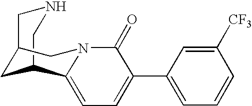 | 96e | 8.3 | >10,000 | 3,700 | >10,000 |
| 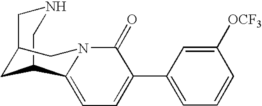 | 97e | 67 | >10,000 | >10,000 | >10,000 |
| 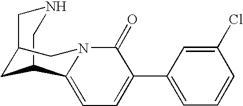 | 98e | 199 | >10,000 | >10,000 | >10,000 |
| 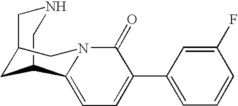 | 99e | 5.7 | >10,000 | 1,200 | >10,000 |
| 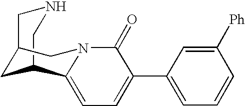 | 100e | 200 | >10,000 | >10,000 | >10.000 |

a) values are the mean from at least n = 3 to 5 independent assays
b) preliminary results
c) show that the group has the same impact on the binding affinity and the compound 96e shows nearly the same Kᵢ value (Kᵢ = 8.3 nM). Substitution of the phenyl ring with larger electron withdrawing nitro (compound 94e) and trifluoromethoxy (compound 97e) groups resulted in less potent ligands (Kᵢ = 23 and 67 nM, respectively), when compared to compounds 96e and 99e.

TABLE 5
Binding affinities of 3-aryl substituted
“all-carbon” derivatives of cytisine 57

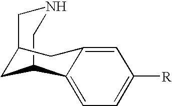

| | |
|---|---|
| 57a R = H | Kᵢ = 34 nM (hα4β2, HEK293 cells) |
| 57k R = 4-PhCF₃ | Kᵢ = 200 nM (hα4β2, HEK293 cells) |
| 57l R = 4-PhOMe | Kᵢ = 370 nM (hα4β2, HEK293 cells) |
| 57j R = Ph | Kᵢ = 500 nM (hα4β2, HEK293 cells) |
| 57m R = 3-PhNH₂ | Kᵢ = 500 nM (hα4β2, HEK293 cells) |

TABLE 6
Structures and Kᵢ values of nitro derivatives of cytisine 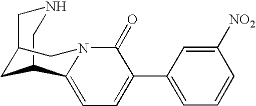
94e Kᵢ = 23 nM (α4β2, rat brain) 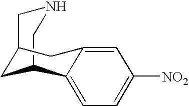
57f Kᵢ = 4.9 nM (α4β2, HEK293 cells) 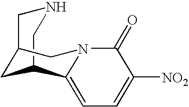
58 Kᵢ = 0.42 nM (α4β2, rat brain)

TABLE 7
Binding affinity values (Kᵢ) for 5-aryl derivatives of cytisine 103e-110e
at α4β2*, α7*, α3β4* and (α1)₂β1γδ nACh receptor subtypes

| | | | | α3β4* | (α1)₂β1γδ |
|---|---|---|---|---|---|
| | | α4β2* | α7* | [³H]epi | [³H]epi |
| | | [³H]epi | [³H]MLA | calf | Torp. calif. |
| | | rat brain | rat brain | adrenals | electroplax |
| Structure | No. | Ki [nM]^{a} | Ki [nM]^{b} | Ki [nM]^{a} | Ki [nM]^{a} |
| 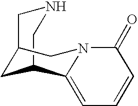 | 27 | 0.122 | 250 | 19 | 1,300 |
| 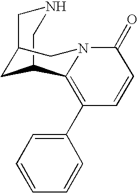 | 103e | 45 | >10,000 | >10,000 | >10,000 |
| 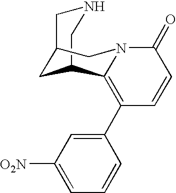 | 104e | 3.7 | >10,000 | 481 | >10,000 |
| 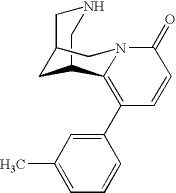 | 105e | 24 | >10,000 | >10,000 | >10,000 |
| 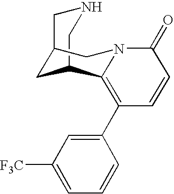 | 106e | 55 | >10,000 | >10,000 | >10,000 |
| 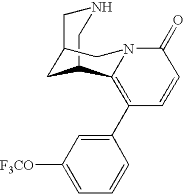 | 107e | 23 | >10,000 | >10,000 | >10,000 |
| 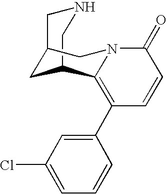 | 108e | 170^{b} | >10,000 | >10,000 | >10,000 |
| 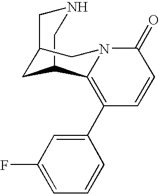 | 109e | 300 | >10,000 | >2,000 | >10,000 |
| 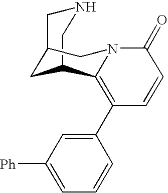 | 110e | 190 | >10,000 | >10,000 | >10,000 |

a) values are the mean from at least n = 3 to 5 independent assays
b) preliminary results

TABLE 8

| | | | | α3β4* | (α1)₂β1γδ |
|---|---|---|---|---|---|
| | | α4β2* | α7* | [³H]epi | [³H]epi |
| | | [³H]epi | [³H]MLA | calf | Torp. calif. |
| | | rat brain | rat brain | adrenals | electroplax |
| Structure | No. | Ki [nM]^{a} | Ki [nM]^{b} | Ki [nM]^{a} | Ki [nM]^{a} |
| 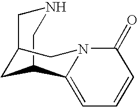 | 27 | 0.122 | 250 | 19 | 1,300 |
| 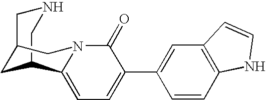 | 117e | 853 | >10,000 | >10,000 | >10,000 |
| 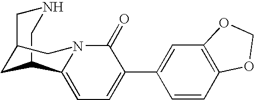 | 118e | 110 | >10,000 | >10,000 | >10,000 |
| 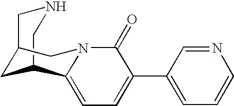 | 119e | 0.91 | >10,000 | 119 | >10,000 |
| 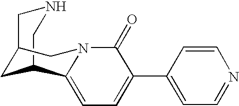 | 120e | 3.9 | >10,000 | 436 | >10,000 |
| 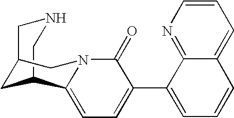 | 121e | 95 | >10,000 | >10,000 | >10,000 |
| 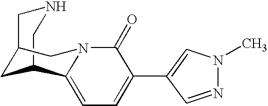 | 122e | 0.177 | >10,000 | 33 | >5,000 |

a) values are the mean from at least n = 3 to 5 independent assays
b) preliminary results

TABLE 9
Binding affinity values (Kᵢ) for 5-heteroaryl derivatives of cytisine 123e-126e
at α4β2*, α7*, α3β4* and (α1)₂β1γδ nACh receptor subtypes

| | | | | α3β4* | (α1)₂β1γδ |
|---|---|---|---|---|---|
| | | α4β2* | α7* | [³H]epi | [³H]epi |
| | | [³H]epi | [³H]MLA | calf | Torp. calif. |
| | | rat brain | rat brain | adrenals | electroplax |
| Structure | No. | Ki [nM]^{a} | Ki [nM]^{b} | Ki [nM]^{a} | Ki [nM]^{a} |
| 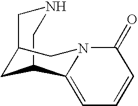 | 27 | 0.122 | 250 | 19 | 1,300 |
| 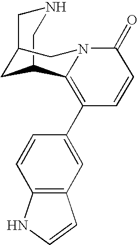 | 123e | 20.4 | >10,000 | >10,000 | >10,000 |
| 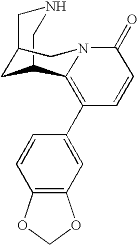 | 124e | 96 | >10,000 | >10,000 | >10,000 |
| 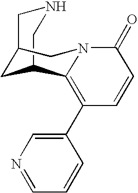 | 125e | 10.9 | >10,000 | 4,300 | >10,000 |
| 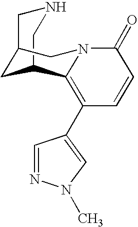 | 126e | 2.2 | >10,000 | 656 | >10,000 |

a) values are the mean from at least n = 3 to 5 independent assays
b) preliminary results

TABLE 10
Binding affinity values (Kᵢ) for distributed derivatives of cytisine 128e and
at α4β2*, α7*, α3β4* and (α1)₂β1γδ nACh receptor subtypes

| | | | | α3β4* | (α1)₂β1γδ |
|---|---|---|---|---|---|
| | | α4β2* | α7* | [³H]epi | [³H]epi |
| | | [³H]epi | [³H]MLA | calf | Torp. calif. |
| | | rat brain | rat brain | adrenals | electroplax |
| Structure | No. | Ki [nM]^{a} | Ki [nM]^{b} | Ki [nM]^{a} | Ki [nM]^{a} |
| 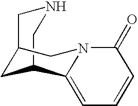 | 27 | 0.122 | 250 | 19 | 1,300 |
| 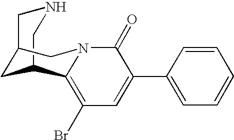 | 128e | 131 | >10,000 | >5,000 | >10,000 |
| 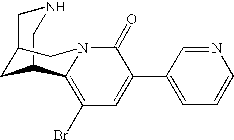 | 129e | 92 | >10,000 | >2,000 | >10,000 |

a) values are the mean from at least n = 3 to 5 independent assays
b) preliminary results

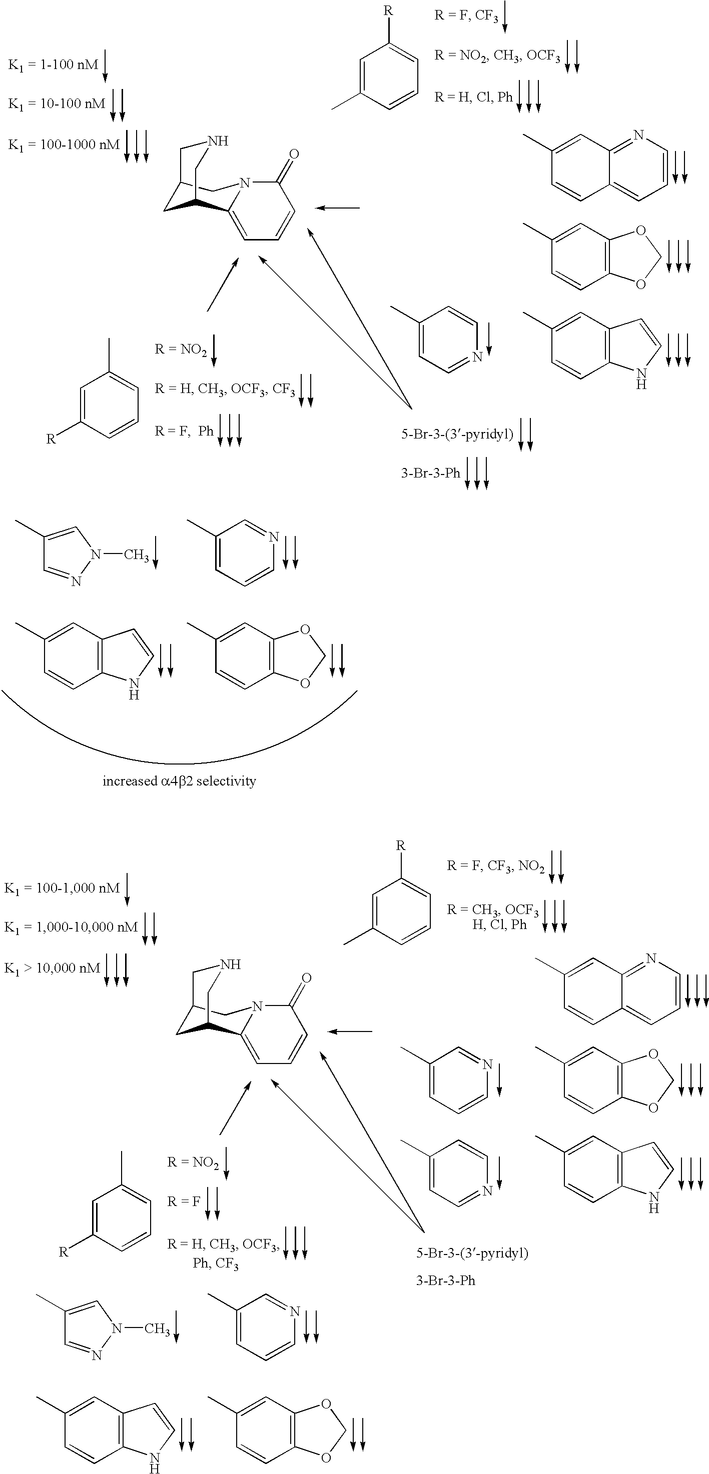

### Antidepressant Effects of Cytisine, a Partial Agonist of Nicotinic Acetylcholine Receptors

In this example, the goal of the testing in vivo (mice) was to determine whether blockade of the high affinity, α4/β2 subclass of nAChRs have anti-depressant-like properties. Additionally, the approach was also to test whether cytisine can potentiate the antidepressant effect of serotonergic activation. An additional goal was to determine the brain regions involved in antidepressant response yielded by nicotinic antagonists.

### Methods

C57BL/6J male mice, 34 months of age were used in these experiments. At least 48 hours between the different tests (as above), there was an acute i.p. injection of cytisine, then the mice were subjected to a tail suspension test (TST) at 6 minutes, and a record of immobility (time and patterns) was provided. At 15 minutes, the mice were subject to a forced swim test (FST) and a recorded of immobility (time and patters) was provided. Control behavior was provided by testing locomotor activity at 20 minutes in an open field.

At 21 days, the mice were injected with an i.p. treatment of cytisine, then the following tests were performed. A novelty-suppressed feeding test (NSFT) was conducted consisting of a 24 hour food deprivation, and a recording of the time to initiate a first feeding episode in an open-field, with controls for appetite and motivation (measure of weight loss and food intake). Control behavior: Anxiety in light/dark box, 6 minutes, measure of time spent in lit side vs. dark side.

Potentiation was determined using cytisine+5HT1A/7 receptor agonist 80HDPAT.

In addition, c-fos immunostaining showed the marker of neuronal activity.

### Conclusions of Testing

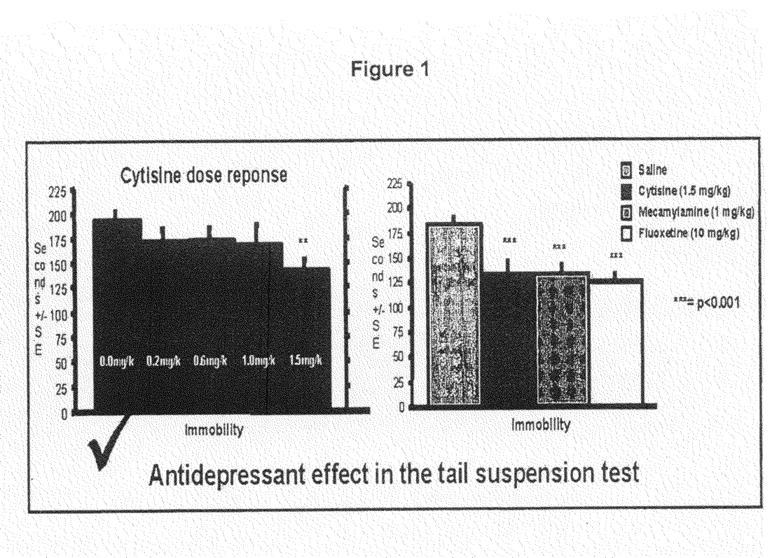

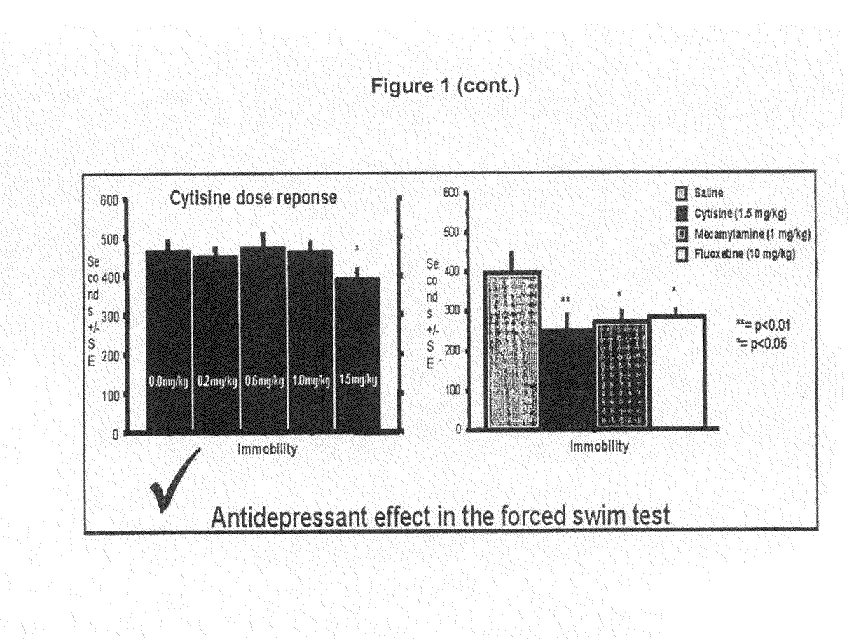

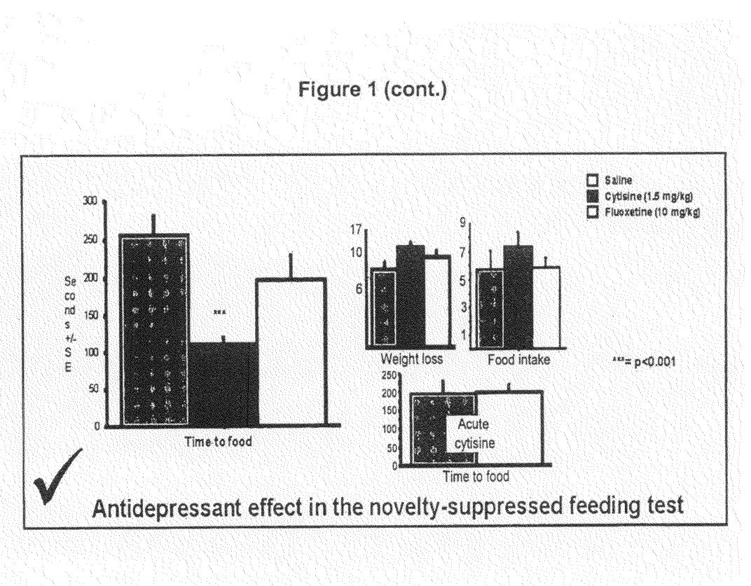

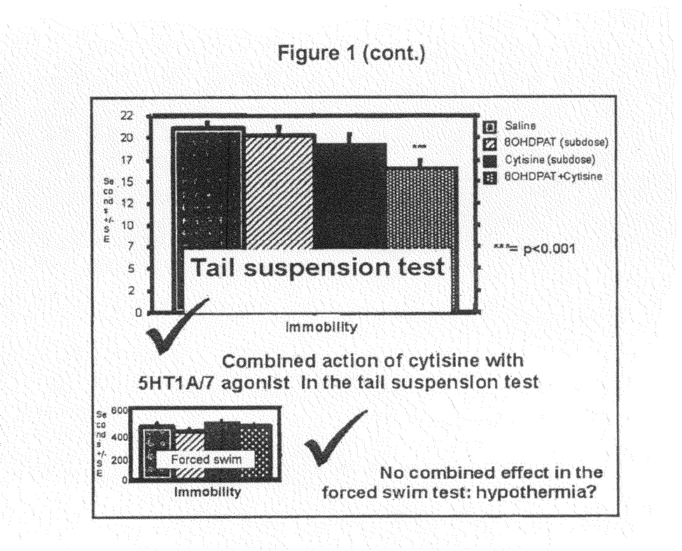

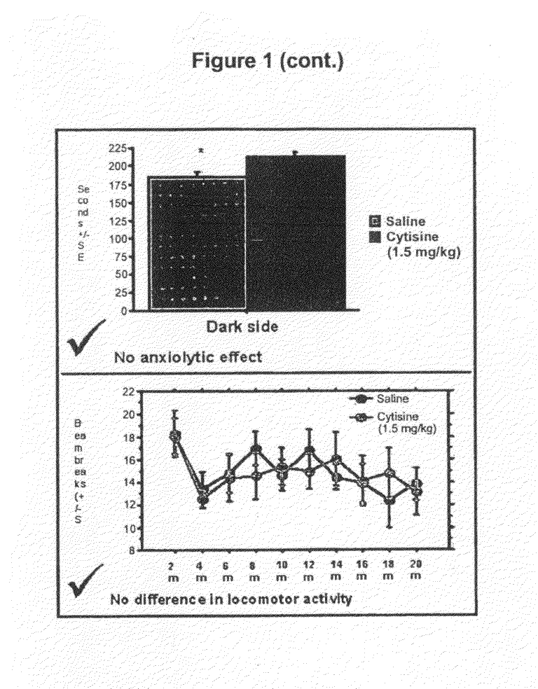

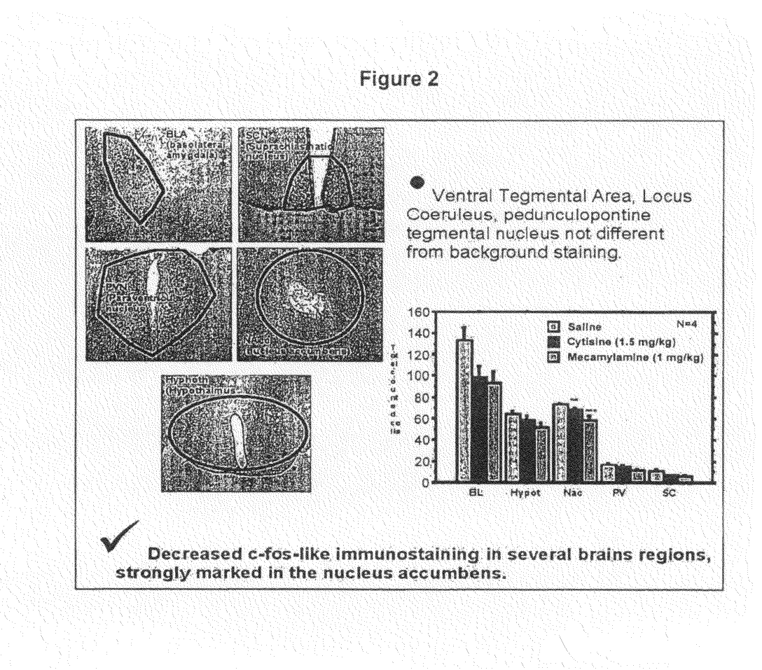

There was shown a generalized antidepressant-like profile of cytisine in three paradigms of antidepressant efficacy (FIG. 1). Blockade of α4/β2 nAChRs yield antidepressant-like activity (FIG. 1). No modification of the locomotor activity was seen. The differences seen in TST and FST were not due to hyperactivity. The difference in NSDT was not due to acute anxiolytic effect. Reductions of neuronal activity in the amygdala, hypothalamus and in the nucleus accumbens was exhibited (FIG. 2). There was seen a potentiation of cytisine effects by the 5HT1A7 agonist 8OHDPAT evidencing that serotoninergic and cholinergic pathways are complementary targets for antidepressant treatments/actions. In addition, cytisine appears to modify food intake, similar to nicotine (agonism of α3β4).

### Biological Evaluation of Particular Cytisine Analogs

Biological evaluation of the compounds on various nicotinic actylcholine receptors (nAChRs) was conducted with cells described below. The affinities of the cytisine analogs for membranes from [₃H]-epibatidine binding in a standard filtration assay format. Functional agonist activity of the cytisines in cells using a fluorescence assay sensitive to changes in cytosolic calcium. The results for the two compounds appear below.

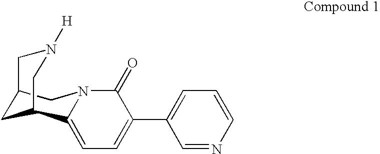

Ki (α4β2 nAChRs): 0.91 nM (rat brain)
Ki (α3β4 nAChRs): 119 nM (calf adrenals)
Ki (Muscle nAChRs): >10,000 nM (Torpedo californica electroplax)

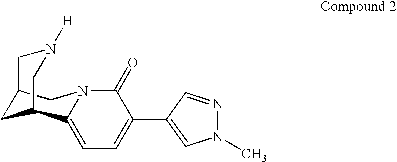

Ki (α4β2 nAChRs): 0.177 nM (rat brain)
Ki (α3β4 nAChRs): 33 nM (calf adrenals)
Ki (Muscle nAChRs): >5,000 nM (Torpedo californica electroplax)

### Animals

Subjects were 12-week old male C57BL/6J mice (The Jackson Laboratory, Bar Harbor Me.). Subjects were housed 5 per cage under standardized conditions (12:12 light cycle with lights on at 7:00 am) and were allowed at least 7 days of rest before testing. Food and water were available ad libitum.

### Cytisine Derivative Administration (Compound 1)

Before each behavioral test, mice were weighed in the holding room, and subsequently injected i.p. with a saline/Compound 1 solution (1.5 mg/kg). Placebo cage-mate controls received an injection of saline.

### Behavioral Assay

The tail suspension test took place between 2:00 pm and 5:00 pm. Animals were injected with compound 1 (above) (0.25 mg/kg) or vehicle (saline) and tested 45 minutes after injection. Immediately after injection, each animal was placed in a clean cage and transferred to the testing room.

Approximately 1 cm from the end, each mouse's tail was taped (Scotch 35 Vinyl Electrical Tape) to a paperclip. Mice were suspended by the tail approximately 80 cm above the floor and the duration of immobility, defined as the absence of all movement except for those required for respiration, was recorded by an observer for 6 minutes. The animals were then returned to the holding room.

### Results

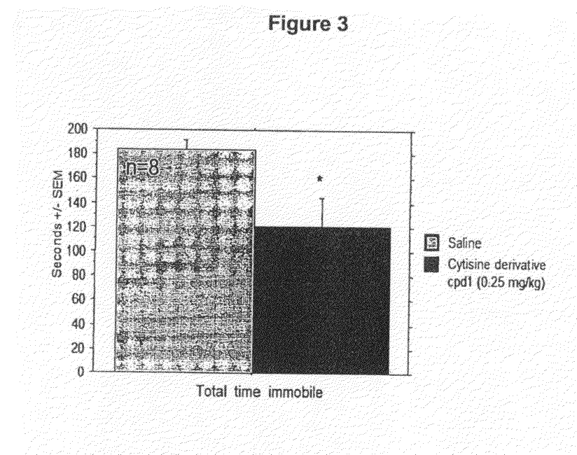

The mean duration of the immobility in the tail suspension test for mice treated acutely with compound 1 is shown in attached FIG. 3. Male mice treated with compound 1 were significantly less mobile than mice receiving a saline injection (p=0.014). For comparison, cytisine shows similar effects at 1 mg/kg whereas lower doses did not yield any significant effects. Additionally, mice treated sub-chronically (3 days) with the classical tricyclic antidepressant amitriptyline in the drinking water showed similar behavior.

1. A compound according to the chemical structure I or III: 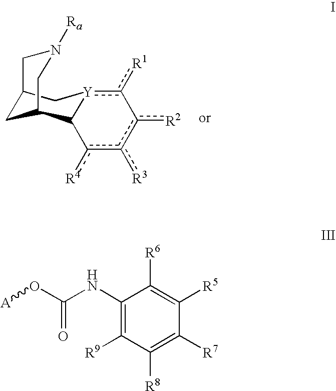
Wherein Rₐ is H, a C₁-C₆ optionally substituted alkyl group, an optionally substituted C₂-C₂₀ acyl (forming an amide) or an optionally substituted carboxyester (forming a urethane with the amine) group;
Y is C—R₁, or N;
R₁ is absent (such that Y forms a double bond with the adjacent carbon atom), H, or an optionally substituted C₁-C₃ alkyl, vinyl or alkynyl group;
Each of R¹, R², R³ and R⁴ is independently O, S (such that the O or S forms a double bond with the adjacent carbon atom), H, NO₂, CN, halogen (F, Br, Cl or I), a C₁-C₆ optionally substituted carboxylic acid group, an optionally substituted O—(C₁-C₆)alkyl (alkoxy), an optionally substituted S—(C₁-C₆)alkyl (thioether), an optionally substituted C₁-C₁₂ hydrocarbyl, an optionally substituted aryl, an optionally substituted heteroaryl, an optionally substituted heterocycle, an optionally substituted —C(O)—(C₁-C₆) alkyl (ketone), an optionally substituted —C(O)—O—(C₁-C₆) alkyl (ester), an optionally substituted O—C(O)—(C₁-C₆) alkyl (ester), an optionally substituted —C(O)—NH(C₁-C₆) alkyl (urea), an optionally substituted —C(O)—N(C₁-C₆)dialkyl, an optionally substituted —C(O)—NH(aryl), an optionally substituted —C(O)—N(diaryl), an optionally substituted —C(O)—NH(heteroaryl), an optionally substituted —C(O)—N(diheteroaryl), an optionally substituted —C(O)—NH(heterocycle), an optionally substituted —C(O)—N(diheterocycle), an optionally substituted —NHC(O)—(C₁-C₆)alkyl, an optionally substituted —NHC(O)-aryl, an optionally substituted —NHC(O)-heteroaryl or an optionally substituted —NHC(O)-heterocycle) with the proviso that not more than two of R¹, R², R³ and R⁴ is O or S;
A is a 5 to 9-membered substituted azacyclic or azabicyclic group, an —NR^{1a}R^{2a}, or a —NR^{1a}R^{2a}R^{3a}+ group;
R^{1a}, R^{2a} and R^{3a} are each independently H or an optionally substituted C₁-C₃ alkyl group;
R⁵ and R⁸ are each independently selected from H, NO₂, CN, halogen, a C₁-C₆ optionally substituted carboxylic acid group, an optionally substituted O—(C₁-C₆)alkyl (alkoxy), an optionally substituted S—(C₁-C₆)alkyl (thioether), an optionally substituted C₁-C₁₂ hydrocarbyl, an optionally substituted aryl, an optionally substituted heteroaryl, an optionally substituted heterocycle, an optionally substituted —C(O)—(C₁-C₆) alkyl (ketone), an optionally substituted —C(O)—O—(C₁-C₆) alkyl (ester), an optionally substituted O—C(O)—(C₁-C₆) alkyl (ester), an optionally substituted —C(O)—NH(C₁-C₆) alkyl (urea), an optionally substituted —C(O)—N(C₁-C₆)dialkyl, an optionally substituted —C(O)—NH(aryl), an optionally substituted —C(O)—N(diaryl), an optionally substituted —C(O)—NH(heteroaryl), an optionally substituted —C(O)—N(diheteroaryl), an optionally substituted —C(O)—NH(heterocycle), an optionally substituted —C(O)—N(diheterocycle), an optionally substituted —NHC(O)—(C₁-C₆)alkyl, an optionally substituted —NHC(O)-aryl, an optionally substituted —NHC(O)-heteroaryl or an optionally substituted —NHC(O)-heterocycle);
R⁶, R⁷ and R⁹ are each independently H, NO₂, CN, halogen or an optionally substituted C₁-C₁₂ hydrocarbyl group or an optionally substituted aryl group,
or a pharmaceutically acceptable salt, solvate or polymorph thereof.

2. The compound of structure I according to claim 1.

3. The compound of structure III according to claim 1.

4. The compound according to claim 1 wherein Rₐ is H, an optionally substituted C₂-C₂₀ acyl (forming an amide) or an optionally substituted carboxyester group.

5. The compound according to claim 1 wherein Y is C—R₁ or N and R₁ is absent.

6. The compound according to claim 2 wherein at least one of R², R³ or R⁴ is other than H and Rₐ is H, an acyl group or carboxyester group.

7. The compound according to claim 2 wherein Rₐ is other than H.

8. The compound according to claim 1 wherein Y is N, R¹ is O, and R² is other than H.

9. The compound according to claim 8 wherein R³ is H or a halogen.

10. The compound according to claim 9 wherein R³ is F, Cl or Br.

11. The compound according to claim 1 wherein R² and R⁴ are independently H or an optionally substituted phenyl or an optionally substituted heterocyclic group.

12. The compound according to claim 11 wherein R² and R⁴ are independently H or an optionally substituted phenyl or optionally substituted benzyl group, with the proviso that one of R² and R⁴ is other than H.

13. The compound according to claim 11 wherein R² or R⁴ is a meta-substituted phenyl or a meta-substituted benzyl group.

14. The compound according claim 11 wherein R² or R⁴ is an optionally substituted heterocyclic group.

15. The compound according to claim 14 wherein said heterocyclic group is morpholine, piperidine, piperazine, furan, thiophene, indole, benzofuran, benzofurazan, pyridine, quinoline, imidazole, diazole or pyrazole group, all optionally substituted.

16. The compound according to claim 15 wherein said pyridine is an optionally substituted 2-pyridyl or 3-pyridyl group.

17. The compound according to claim 15 wherein said furan group is an optionally substituted 2-furanyl or 3-furanyl group.

18. The compound according to claim 14 wherein R² is an optionally substituted group according to the structure:

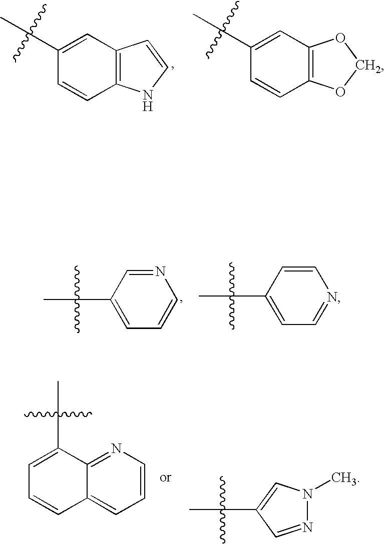

19. The compound according to claim 1 which is

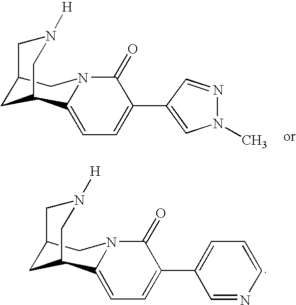

20.-39. (canceled)

40. A pharmaceutical composition comprising an effective amount of a compound according to claim 1 in combination with a pharmaceutically acceptable carrier additive or excipient.

41. A pharmaceutical composition comprising an effective amount of a compound according to claim 1 in combination with at least one additional agent selected from the group consisting of tricyclic antidepressants, MAO inhibitors and serotonin reuptake inhibitors, further in combination with a pharmaceutically acceptable carrier additive or excipient.

42.-44. (canceled)

45. A method of modulating a nicotinic acetylcholine receptor (nAChR) in a patient comprising administering to said patient an effective amount of a compound according to claim 1.

46. The method according to claim 45 wherein said nicotinic acetylcholine receptor is α4β2 nAChR, α3β4 nAChR or α7 nAChR.

47. The method according to claim 45 wherein said nicotinic acetylcholine receptor is α4β2 nAChR.

48.-49. (canceled)

50. A method of treating a mood disorder in a patient comprising administering to said patient an effective amount of a compound according to claim 1.

51. The method according to claim 47 wherein said mood disorder is major depressive disorder, bipolar disorder, unipolar disorder, dysthymia (dysthymic disorder), post-partum depression, seasonal affective disorder or schizoaffective disorder

52.-61. (canceled)
